(19) United States
(12) Patent Application Publication
(10) Pub. No.: US 2004/0248103 A1
(43) Pub. Date: Dec. 9, 2004

(54) Proximity-mediated rolling circle amplification

(76) Inventors: William John Feaver, Branford, CT (US); Michael Mullenix, Branford, CT (US)

Correspondence Address: NEEDLE & ROSENBERG, P.C., SUITE 1000, 999 PEACHTREE STREET, ATLANTA, GA, 30309-3915, US

(21) Appl. No.: 10/454,946

(22) Filed: Jun. 4, 2003

(51) Int. Cl. C12Q 1/68; C12P 19/34

(52) U.S. Cl. 435/6

(57) ABSTRACT

Disclosed are compositions and methods for proximity-mediated rolling circle amplification and for real-time detection of proximity-mediated rolling circle amplification products. Rolling circle amplification (RCA) refers to nucleic acid amplification reactions involving replication of a circular nucleic acid template to form a long strand with tandem repeats of the sequence complementary to the circular template. In proximity-mediated RCA, binding guide conjugates are brought into close proximity, generally by associating them to the same analyte or to two analytes in close proximity. The binding guide conjugates comprise a specific binding molecule and a guide oligonucleotide. The guide oligonucleotides are complementary to guide complement portions on half circle probes. The complementary sequences between the guide oligonucleotides and half circle probes allow both ends of each half circle probe to hybridize adjacent to an end of the other half circle probe and to be ligated together to form a circular nucleic acid molecule comprising the two half circle probes. This circular nucleic acid molecule can then be used as the template in RCA.

### FIELD OF THE INVENTION

The disclosed invention is generally in the field of nucleic amplification and detection and specifically in the area of detection of rolling circle amplification products during amplification.

### BACKGROUND OF THE INVENTION

Numerous nucleic acid amplification techniques have been devised, including strand displacement cascade amplification (SDCA) (referred to herein as exponential rolling circle amplification (ERCA)) and rolling circle amplification (RCA) (U.S. Pat. No. 5,854,033; PCT Application No. WO 97/19193; Lizardi et al., Nature Genetics19(3):225-232 (1998)); multiple displacement amplification (MDA) (PCT Application WO 99/18241); strand displacement amplification (SDA) (Walker et al., Nucleic Acids Research 20:1691-1696 (1992), Walker et al., Proc. Natl. Acad. Sci. USA 89:392-396 (1992)); polymerase chain reaction (PCR) and other exponential amplification techniques involving thermal cycling, self-sustained sequence replication (3SR), nucleic acid sequence based amplification (NASBA), and amplification with Qβ replicase (Birkenmeyer and Mushahwar, J. Virological Methods 35:117-126 (1991); Landegren, Trends Genetics 9:199-202 (1993)); and various linear amplification techniques involving thermal cycling such as cycle sequencing (Craxton et al., Methods Companion Methods in Enzymology 3:20-26 (1991)).

Rolling Circle Amplification (RCA) driven by DNA polymerase can replicate circular oligonucleotide probes with either linear or geometric kinetics under isothermal conditions (Lizardi et al., Nature Genet. 19: 225-232 (1998); U.S. Pat. Nos. 5,854,033 and 6,143,495; PCT Application No. WO 97/19193). If a single primer is used, RCA generates in a few minutes a linear chain of hundreds or thousands of tandemly-linked DNA copies of a target that is covalently linked to that target. Generation of a linear amplification product permits both spatial resolution and accurate quantitation of a target. DNA generated by RCA can be labeled with fluorescent oligonucleotide tags that hybridize at multiple sites in the tandem DNA sequences. RCA can be used with fluorophore combinations designed for multiparametric color coding (PCT Application No. WO 97/19193), thereby markedly increasing the number of targets that can be analyzed simultaneously. RCA technologies can be used in solution, in situ and in microarrays. In solid phase formats, detection and quantitation can be achieved at the level of single molecules (Lizardi et al., 1998). Ligation-mediated Rolling Circle Amplification (LM-RCA) involves circularization of a probe molecule hybridized to a target sequence and subsequent rolling circle amplification of the circular probe (U.S. Pat. Nos. 5,854,033 and 6,143,495; PCT Application No. WO 97/19193). Very high yields of amplified products can be obtained with exponential rolling circle amplification (U.S. Pat. Nos. 5,854,033 and 6,143,495; PCT Application No. WO 97/19193) and multiply-primed rolling circle amplification (Dean et al., Genome Research 11:1095-1099 (2001)).

### BRIEF SUMMARY OF THE INVENTION

Disclosed are compositions and methods for proximity-mediated rolling circle amplification and for real-time detection of proximity-mediated rolling circle amplification products. Rolling circle amplification (RCA) refers to nucleic acid amplification reactions involving replication of a circular nucleic acid template (referred to as an amplification target circle; ATC) to form a long strand (referred to as tandem sequence DNA; TS-DNA) with tandem repeats of the sequence complementary to the circular template. Proximity-mediated rolling circle amplification is a type of rolling circle amplification whereby the process is mediated in some way by the proximity or spatial relationship of various molecules involved in the amplification. Generally, proximity-mediated RCA can be accomplished by using the proximity of certain molecules or moieties as a condition that affects the formation of an amplification target circle.

In some forms, the disclosed method involves association of binding guide conjugates to the same analyte or to two analytes in close proximity. This brings the binding guide conjugates into close proximity. In these forms of the method, the binding guide conjugates comprise a specific binding molecule and a guide oligonucleotide. The guide oligonucleotides are complementary to guide complement portions on half circle probes. The complementary sequences between the guide oligonucleotides and half circle probes generally can be chosen such that two half circle probes each are complementary to both of the guide oligonucleotides in the binding guide conjugates that are in close proximity. In particular, this arrangement can allow both ends of each half circle probe to hybridize adjacent to an end of the other half circle probe and to be ligated together to form a circular nucleic acid molecule comprising the two half circle probes. In the method, this circularization generally will take place only when the binding guide conjugates are bound to analytes that bring them into close proximity. In an assay, if there is no analyte present, the binding guide conjugates will not be brought into close proximity and the half circle probes will not be circularized. The amount of circularized half circle probes formed can be a measure of the amount of analyte in a sample.

In other forms of the disclosed method, binding guide conjugates comprise a half circle probe and a guide oligonucleotide. The binding guide conjugates are associated with the same analyte or to two analytes in close proximity, bringing the binding guide conjugates into close proximity. The guide complement portions on the half circle probes are complementary to guide oligonucleotides. The complementary sequences between the guide oligonucleotides and half circle probes generally can be chosen such that the guide oligonucleotides each are complementary to both half circle probes in the binding guide conjugates that are in close proximity. In particular, this arrangement can allow both ends of each half circle probe to hybridize adjacent to an end of the other half circle probe and to be ligated together to form a circular nucleic acid molecule comprising the two half circle probes. In the method, this circularization generally will take place only when the binding guide conjugates are bound to analytes that bring them into close proximity. In an assay, if there is no analyte present, the binding guide conjugates will not be brought into close proximity and the half circle probes will not be circularized. The amount of circularized half circle probes formed can be a measure of the amount of analyte in a sample.

These forms of the disclosed method also can be performed in a competitive assay mode. In this mode, the assay is carried out in the presence of analyte(s) immobilized on a solid support. The immobilized analytes are the same as the analytes to be assayed. This analyte competes for analyte present in a sample being assayed. In the absence of analyte in the sample, the specific binding molecules of the binding guide conjugates can associate with the immobilized analyte(s), thus bringing the binding guide conjugates and their guide oligonucleotides into close proximity. This allows circularization of half circle probes as described above. In the presence of analyte in the sample, the specific binding molecules of the binding guide conjugates can associate with analyte in the sample thus keeping the binding guide conjugates from associating with the analyte in the second binding guide conjugate. As a result, the binding guide conjugates generally will bind to different antigens in solution and will not be in close proximity, which prevents the half circle probes from being circularized. The effect of any binding guide conjugates that do bind to the same antigen (bringing them into close proximity) can be eliminated by washing to remove materials not associated with the solid support. In an assay, the competition between sample analytes and analytes in binding guide conjugates can result in either elimination of circle formation or a reduction of circle formation, depending on the relative concentrations of the second binding guide conjugate and analyte in the sample. Thus, the amount of circularized half circle probes formed can be a measure of the concentration of analyte in a sample. This competitive assay mode can also be performed with the competitive analyte free in solution rather than immobilized. The analyte used to compete with analyte in a sample can be referred to as a test analyte.

In other forms of the disclosed method, two forms of binding guide conjugate are used. One form of binding guide conjugate comprises a specific binding molecule and a guide oligonucleotide. The other form of binding guide conjugate, which can be referred to as a binding guide analyte, comprises an analyte and a guide oligonucleotide. The specific binding molecule of the first binding guide conjugate is specific for the analyte in the second binding guide conjugate. These forms of binding guide conjugates can be used to detect the same type of analyte in a sample by a competitive mechanism. In the absence of analyte in the sample, the specific binding molecule of the first binding guide conjugate can associate with the analyte in the second binding guide conjugate, thus bringing the binding guide conjugates and their guide oligonucleotides into close proximity. The guide oligonucleotides are complementary to guide complement portions on half circle probes. The complementary sequences between the guide oligonucleotides and half circle probes generally can be chosen such that two half circle probes each are complementary to both of the guide oligonucleotides in the binding guide conjugates that are in close proximity. In particular, this arrangement can allow both ends of each half circle probe to hybridize adjacent to an end of the other half circle probe and to be ligated together to form a circular nucleic acid molecule comprising the two half circle probes. This circularization generally will take place only when the first binding guide conjugate is bound to the analyte in the second binding guide conjugate (which brings the binding guide conjugates into close proximity.

In the presence of analyte in the sample, the specific binding molecule of the first binding guide conjugate can associate with analyte in the sample thus keeping the first binding guide conjugate from associating with the analyte in the second binding guide conjugate. As a result, the first and second binding guide conjugates will not be in close proximity and the half circle probes will not be circularized. In an assay, the competition between sample analytes and analytes in binding guide conjugates can result in either elimination of circle formation or a reduction of circle formation, depending on the relative concentrations of the second binding guide conjugate and analyte in the sample. Thus, the amount of circularized half circle probes formed can be a measure of the concentration of analyte in a sample. These forms of the method can also be performed with binding guide conjugates having half circle probes instead of guide oligonucleotides.

Both the first and second binding guide conjugates can be used free in solution. Alternatively, either the first binding guide conjugate or the second binding guide conjugate can be immobilized on a solid support. In this case, analyte in a sample prevents or reduces association of the first binding guide conjugates with the solid support (via the specific binding molecule of an immobilized first binding guide conjugate) or prevents or reduces association of the second binding guide conjugate with the solid support (via the analyte of the immobilized second binding guide conjugate).

The disclosed methods can involve components that are all in solution or with some components immobilized. Some forms of the method performed all in solution can be performed as homogeneous assays where the assay components and the samples can be mixed and the assay performed without washing, separation or purification. this can greatly simplify the method resulting in a highly efficient assay suitable for high throughput applications.

The analytes can be any molecule or moiety of interest. For example, proteins and peptides, nucleic acids, and other biological molecules are useful analytes. Nucleic acids can be detected using binding guide conjugates that use, for example, an oligonucleotide for the specific binding molecule. Because both the specific binding molecule and the guide oligonucleotide can be oligonucleotides in such binding guide conjugates, the guide oligonucleotides can be, for example, a single oligonucleotide. Binding guide conjugates for detection of nucleic acids can be brought into close proximity by, for example, choosing guide oligonucleotide sequence that is complementary to sequences in a nucleic acid molecule of interest that close together. Nucleic acids can also be detected using binding guide conjugates having specific binding molecules that bind to haptens or other labeling moieties that can be incorporated into or attached to nucleic acid molecules. A pair of binding guide conjugates can also be directed to different types of binding targets in nucleic acid molecules. For example, one binding guide conjugate can have a nucleic acid probe as the specific binding molecule and the other can have a specific binding molecule that binds to a hapten or label. Binding guide conjugates in a pair can be directed to different types and classes of analytes. All that is required for detection using the disclosed methods is for the analytes to be in close proximity.

Real-time detection is detection that takes place during the amplification reaction or operation. Generally, such detection can be accomplished by detecting amplification product at one or more discrete times during amplification, continuously during all or one or more portions of the amplification, or a combination of discrete times and continuous detection. Real-time detection can be aided by the use of labels or moieties that embody or produce a detectable signal that can be detected without disrupting the amplification reaction or operation. Fluorescent labels are an example of useful labels for real-time detection. A particularly useful means of obtaining real-time detection is the use of fluorescent change probes and/or primers in the amplification operation. With suitably designed fluorescent change probes and primers, fluorescent signals can be generated as amplification proceeds. In most such cases, the fluorescent signals will be in proportion to the amount of amplification product and/or amount of target sequence or target molecule.

In some forms, the disclosed method involves proximity-mediated rolling circle amplification and real-time detection of amplification products where amplification includes multiply-primed rolling circle amplification (MPRCA). Rolling circle replication can be primed at one or more sites on the circular template. Multiply-primed RCA refers to RCA where replication is primed at a plurality of sites on the circular template. Multiply-primed RCA increases the sensitivity of singly-primed rolling circle amplification. Rolling circle amplification refers both to rolling circle replication and to processes involving both rolling circle replication and additional forms of amplification (such as replication of tandem sequence DNA).

Multiply-primed RCA can be performed using a single primer (which hybridizes to multiple sites on the amplification target circle) or multiple primers (each of which can hybridize to a single site on the amplification target circle or multiple sites on the amplification target circle). Multiple priming (as occurs in MPRCA) can increase the yield of amplified product from RCA. Primers anneal to multiple locations on the circular template and a product of extension by polymerase is initiated from each location. In this way, multiple extensions are achieved simultaneously from a single amplification target circle.

In some forms of the disclosed method, multiple priming can be achieved in several different ways. For example, two or more specific primers that anneal to different sequences on the circular template can be used, one or more specific primers that each anneals to a sequence repeated at two or more separate locations on the circular template can be used, a combination of primers that each anneal to a different sequence on the circular template or to a sequence repeated at two or more separate locations on the circular templates can be used, one or more random or degenerate primers, which can anneal to many locations on the circle, can be used, or a combination of such primers can be used.

Fluorescent change probes and primers, which are useful for obtaining real-time detection of amplification, refer to all probes and primers that involve a change in fluorescence intensity or wavelength based on a change in the form or conformation of the probe or primer and nucleic acid to be detected, assayed or replicated. Examples of fluorescent change probes and primers include molecular beacons, Amliplifluors, FRET probes, cleavable FRET probes, TaqMan probes, scorpion primers, fluorescent triplex oligos, including but not limited to triplex molecular beacons or triplex FRET probes, fluorescent water-soluble conjugated polymers, PNA probes and QPNA probes. Change in fluorescence wavelength or intensity from fluorescent change probes and primers generally involves energy transfer and/or quenching. Fluorescent change probes and primers can be classified according to their structure and/or function. Fluorescent change probes include, for example, hairpin quenched probes, cleavage quenched probes, cleavage activated probes, and fluorescent activated probes.

Also disclosed are methods of producing small circular single-stranded nucleic acid molecules, such as amplification target circles. The method generally involves hybridization of two or more half circle probes with two or more guide oligonucleotides in close proximity and ligation of the ends of the half circle probes. The guide oligonucleotides are complementary to guide complement portions on the half circle probes. The complementary sequences between the guide oligonucleotides and half circle probes generally can be chosen such that two half circle probes each are complementary to both of the guide oligonucleotides in the binding guide conjugates that are in close proximity. In particular, this arrangement can allow both ends of each half circle probe to hybridize adjacent to an end of the other half circle probe and to be ligated together to form a circular nucleic acid molecule comprising the two half circle probes. The guide oligonucleotides for production of circular nucleic acid molecules can be brought into close proximity in any suitable manner such as those disclosed elsewhere herein. The guide oligonucleotides also can be coupled together, part of the same molecule, or parts of a single oligonucleotide (which has the effect of placing them in close proximity). Prior methods of small circle production generally involve ligation of the two ends of a single oligonucleotide. This requires synthesis of a full length oligonucleotide. In the disclosed method, two or more shorter oligonucleotides (that is, half circle probes) can be used. Despite involving ligation of multiple molecules, the disclosed method is efficient because the guide oligonucleotides used to mediate ligation and circularization of the half circle probes are in close proximity which brings the multiple ends of the half circle probes into ligatable proximity.

Additional advantages of the disclosed method and compositions will be set forth in part in the description which follows, and in part will be understood from the description, or can be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 is a schematic of proximity-mediated rolling circle amplification. A pair of binding guide conjugates, each with a specific binding molecule (SBM1 and SBM2) and guide oligonucleotide (G1 and G2), are bound to an analyte and in close proximity. A pair of half circle probes (HCP1 and HCP2) are hybridized to the guide oligonucleotides (G1 and G2) of each binding guide conjugate. Ligation of the half circle probe pair (HCP1 and HCP2) results in an amplification target circle (ATC), which can then undergo exponential rolling circle amplification (eRCA).

FIG. 2 is a diagram of two binding guide conjugates (BGC1 and BGC2) that are not in close proximity to each other because they are either unbound and/or not specifically bound to an analyte. In the presence of excess half circle probes (HCP1 and HCP2), double ligation of a pair of half circle probes to form an amplification target circle will be blocked because the ends of the HCPs in a pair are unable to hybridize to guide oligonucleotides (e.g., G1 and G2) on another binding guide conjugate since they are hybridized to another pair of HCPs. Only when both binding guide conjugates are in close proximity would pair of half circle probes be likely to anneal as in FIG. 1.

FIG. 3 is a diagram of a pair of binding guide conjugates, each with a specific binding molecule (SBM1 and SBM2) and tethered half circle probe (HCP1 and HCP2), bound to an analyte and in close proximity. Guide oligonucleotides (G1 and G2) are hybridized to the guide complement portions on the pair of half circle probes (HCP1 and HCP2).

FIG. 4 is a diagram of two binding guide conjugates (BGC1 and BGC2) that are not in close proximity to each other because they are either unbound and/or not specifically bound to an analyte. In the presence of excess guide oligonucleotides (G1 and G2), ligation and further annealing of the HCPs to form an amplification target circle will not occur because the ends of a HCP of one binding guide conjugate (e.g., HCP1) are blocked from interacting with the ends of a half circle probe on another binding guide conjugate (e.g., HCP2). Only when both binding guide conjugates are in close proximity would both half circle probes (HCP1 and HCP2) be likely to anneal as in FIG. 3.

FIG. 5 is a diagram of a pair of guide oligonucleotides (G1 and G2) connected to each other by a tether. A pair of half circle probes (HCP1 and HCP2) are hybridized to the pair of guide oligonucleotides.

FIG. 6 is a diagram of a pair of binding guide probes, each with an oligonucleotide or oligonucleotide derivative as the specific binding molecule (SBM1 and SBM2) and guide oligonucleotide (G1 and G2), bound to a target sequence and in close proximity. A pair of half circle probes (HCP1 and HCP2) are hybridized to the guide oligonucleotides (G1 and G2) of each binding guide probe.

FIG. 7 is a diagram of a pair of binding guide probes, each with an oligonucleotide or oligonucleotide derivative as the specific binding molecule (SBM1 and SBM2) and a tethered half circle probe (HCP1 and HCP2), bound to a target sequence and in close proximity. A pair of guide oligonucleotides (G1 and G2) are hybridized to the half circle probes (HCP1 and HCP2) of each binding guide probe.

FIG. 8 is a diagram of two binding guide probes (BGP1 and BGP2) that are not in close proximity to each other because they are either unbound and/or not specifically bound to a target sequence. In the presence of excess guide oligonucleotides (G1 and G2), ligation and further annealing of the HCPs to form an amplification target circle will not occur because the ends of a HCP of one binding guide probe (e.g., HCP1) are blocked from interacting with the ends of a half circle probe on another binding guide probe (e.g., HCP2). Only when both guide probes are in close proximity would both half circle probes (HCP1 and HCP2) be likely to anneal as in FIG. 7.

FIG. 9 is a diagram of a binding guide probe (BGP), which comprises an oligonucleotide or oligonucleotide derivative as a specific binding molecule and a guide oligonucleotide, and a binding guide conjugate (BGC), which comprises a specific binding molecule (specific for a hapten) and a guide oligonucleotide. The specific binding molecules are bound to analytes (a nucleic acid sequence for the BGP and a hapten for the BGC) and in close proximity. A pair of half circle probes are hybridized to the guide oligonucleotides in the binding guide probe and the binding guide conjugate.

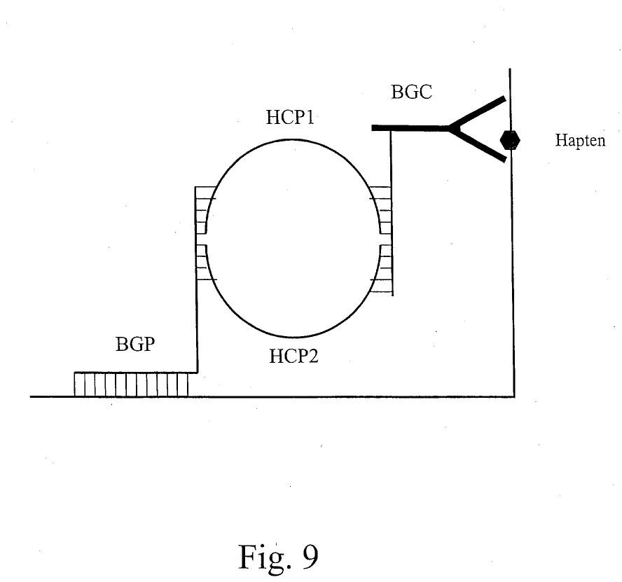

FIGS. 10A and 10B are diagrams of one example of the disclosed method used in a competitive mode. In this example, a binding guide conjugate (BGC; comprising a specific binding molecule (SBM) and a guide oligonucleotide (G1)) and binding guide analyte (BGA; comprising an analyte (Labeled Analyte) and a guide oligonucleotide (G2)) are used. The binding guide analyte is immobilized on a solid support via an antibody. FIG. 10A depicts the association of binding guide conjugate, binding guide analyte and two half circle probes (HCP1 and HCP2) in the absence of analyte in a sample. FIG. 10B depicts the association of binding guide analyte and two half circle probes (HCP1 and HCP2) and the association of a free analyte (Sample Analyte), binding guide conjugate and two half circle probes (HCP1 and HCP2). These associations occur when analyte is present in a sample. The free analyte competes against the analyte in the binding guide analyte for association with the specific binding molecule in the binding guide conjugate. In the presence of analyte, the half circle probes do not hybridize to two guide oligonucleotides and so are not circularized.

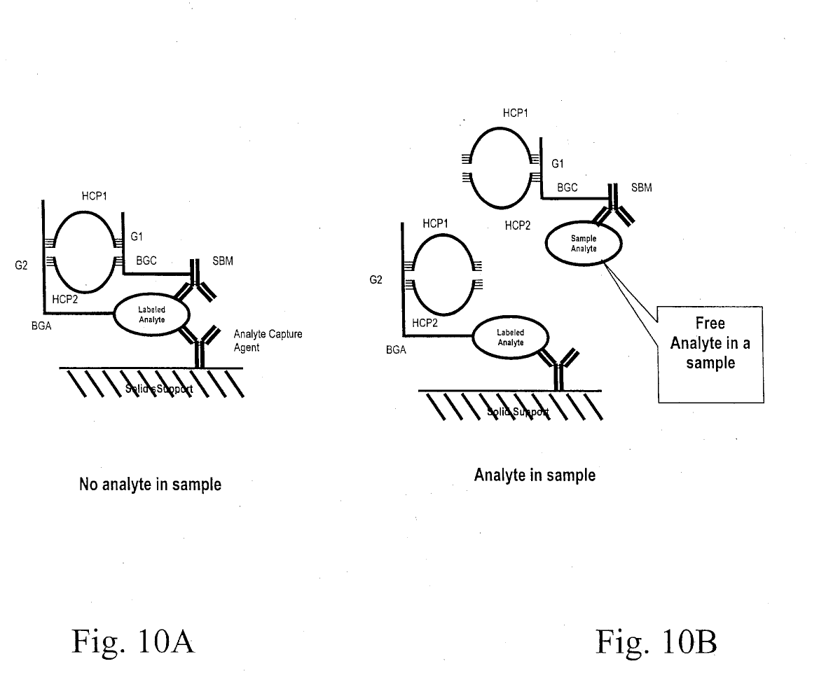

FIGS. 11A and 11B are diagrams of one example of the disclosed method used in a competitive mode. In this example, a binding guide conjugate (BGC; comprising a specific binding molecule (SBM) and a guide oligonucleotide (G1)) and binding guide analyte (BGA; comprising an analyte (Labeled Analyte) and a guide oligonucleotide (G2)) are used. The binding guide analyte is free in solution. FIG. 11A depicts the association of binding guide conjugate, binding guide analyte and two half circle probes (HCP1 and HCP2) in the absence of analyte in a sample. FIG. 11B depicts the association of binding guide analyte and two half circle probes (HCP1 and HCP2) and the association of a free analyte (Sample Analyte), binding guide conjugate and two half circle probes (HCP1 and HCP2). These associations occur when analyte is present in a sample. The free analyte competes against the analyte in the binding guide analyte for association with the specific binding molecule in the binding guide conjugate. In the presence of analyte, the half circle probes do not hybridize to two guide oligonucleotides and so are not circularized.

FIGS. 12A and 12B are diagrams of one example of the disclosed method used in a competitive mode. In this example, two binding guide conjugates (BGC1 and BGC2; each comprising a specific binding molecule (SBM1 and SBM2) and a guide oligonucleotide (G1 and G2)) are used. Competitive analyte (Analyte) is immobilized on a solid support. FIG. 12A depicts the association of binding guide conjugates, the immobilized analyte and two half circle probes (HCP1 and HCP2) in the absence of analyte in a sample. FIG. 12B depicts the association of one binding guide conjugate (BGC1) and two half circle probes (HCP1 and HCP2), the association of the other binding guide conjugate (BGC2) and two half circle probes (HCP1 and HCP2), and the association of a free analyte (Sample Analyte), the two binding guide conjugates (BGC1 and BGC2) and two half circle probes (HCP1 and HCP2). These associations occur when analyte is present in a sample. The free analyte competes against the immobilized analyte for association with the specific binding molecules in the binding guide conjugates. A washing step can be used to distinguish immobilized half circle probe pairs and half circle probe pairs that form in solution.

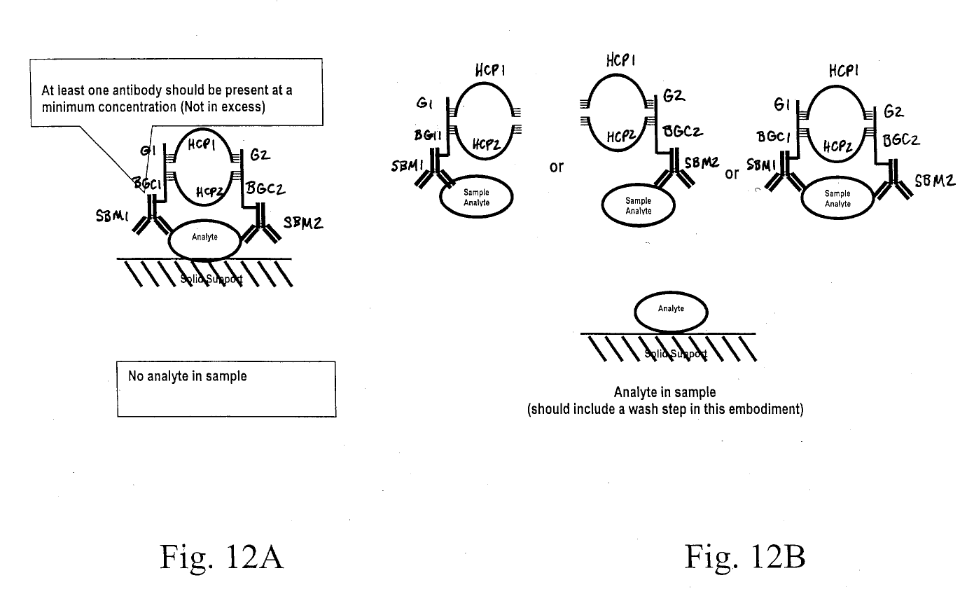

FIGS. 13A and 13B are diagrams of one example of the disclosed method used in a competitive mode. In this example, a binding guide conjugate (BGC; comprising a specific binding molecule (SBM) and a guide oligonucleotide (G1)) and binding guide analyte (BGA; comprising an analyte (Labeled Analyte) and a guide oligonucleotide (G2)) are used. The binding guide conjugate is immobilized on a solid support. FIG. 13A depicts the association of binding guide conjugate, binding guide analyte and two half circle probes (HCP1 and HCP2) in the absence of analyte in a sample. FIG. 13B depicts the association of binding guide analyte and two half circle probes (HCP1 and HCP2) and the association of a free analyte (Sample Analyte), binding guide conjugate and two half circle probes (HCP1 and HCP2). These associations occur when analyte is present in a sample. The free analyte competes against the analyte in the binding guide analyte for association with the specific binding molecule in the binding guide conjugate. In the presence of analyte, the half circle probes do not hybridize to two guide oligonucleotides and so are not circularized.

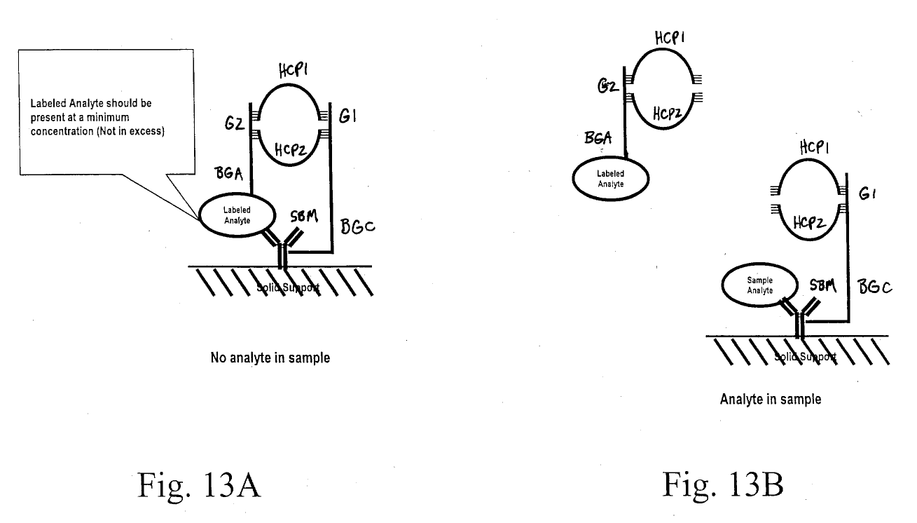

FIGS. 14A and 14B are diagrams of one example of the disclosed method used in a competitive mode. In this example, two binding guide conjugates (BGC1 and BGC2; each comprising a specific binding molecule (SBM1 and SBM2) and a guide oligonucleotide (G1 and G2)) are used. Competitive analyte (Test Analyte) is free in solution, generally in low concentration. FIG. 14A depicts the association of binding guide conjugates, the test analyte and two half circle probes (HCP1 and HCP2) in the absence of analyte in a sample. FIG. 14B depicts the association of one binding guide conjugate (BGC1) and two half circle probes (HCP1 and HCP2), the association of the other binding guide conjugate (BGC2) and two half circle probes (HCP1 and HCP2), and the association of a free analyte (Sample Analyte), the two binding guide conjugates (BGC1 and BGC2) and two half circle probes (HCP1 and HCP2). These associations occur when analyte is present in a sample. The free analyte competes against the test analyte for association with the specific binding molecules in the binding guide conjugates.

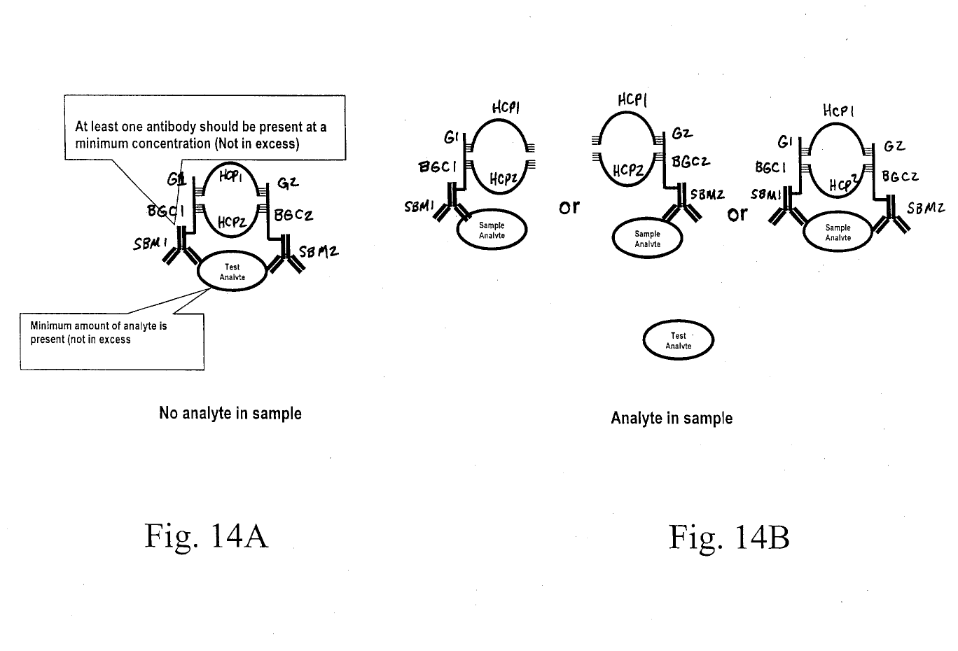

### DETAILED DESCRIPTION OF THE INVENTION

The disclosed method and compositions can be understood more readily by reference to the following detailed description of particular embodiments and the Example included therein and to the Figures and their previous and following description.

Disclosed are compositions and methods for proximity-mediated rolling circle amplification and for real-time detection of proximity-mediated rolling circle amplification products. Rolling circle amplification (RCA) refers to nucleic acid amplification reactions involving replication of a circular nucleic acid template (referred to as an amplification target circle; ATC) to form a long strand (referred to as tandem sequence DNA; TS-DNA) with tandem repeats of the sequence complementary to the circular template. Proximity-mediated rolling circle amplification is a type of rolling circle amplification whereby the process is mediated in someway by the proximity or spatial relationship of various molecules involved in the amplification. Generally, proximity-mediated RCA can be accomplished by using the proximity of certain molecules or moieties as a condition that affects the formation of an amplification target circle.

In some forms, the disclosed method involves association of binding guide conjugates to the same analyte or to two analytes in close proximity. This brings the binding guide conjugates into close proximity. In these forms of the method, the binding guide conjugates comprise a specific binding molecule and a guide oligonucleotide. The guide oligonucleotides are complementary to guide complement portions on half circle probes. The complementary sequences between the guide oligonucleotides and half circle probes generally can be chosen such that two half circle probes each are complementary to both of the guide oligonucleotides in the binding guide conjugates that are in close proximity. In particular, this arrangement can allow both ends of each half circle probe to hybridize adjacent to an end of the other half circle probe and to be ligated together to form a circular nucleic acid molecule comprising the two half circle probes. In the method, this circularization generally will take place only when the binding guide conjugates are bound to analytes that bring them into close proximity. In an assay, if there is no analyte present, the binding guide conjugates will not be brought into close proximity and the half circle probes will not be circularized. The amount of circularized half circle probes formed can be a measure of the amount of analyte in a sample.

Real-time detection is detection that takes place during the amplification reaction or operation. Generally, such detection can be accomplished by detecting amplification product at one or more discrete times during amplification, continuously during all or one or more portions of the amplification, or a combination of discrete times and continuous detection. Real-time detection can be aided by the use of labels or moieties that embody or produce a detectable signal that can be detected without disrupting the amplification reaction or operation. Fluorescent labels are an example of useful labels for real-time detection. A particularly useful means of obtaining real-time detection is the use of fluorescent change probes and/or primers in the amplification operation. With suitably designed fluorescent change probes and primers, fluorescent signals can be generated as amplification proceeds. In most such cases, the fluorescent signals will be in proportion to the amount of amplification product and/or amount of target sequence or target molecule.

In some forms, the disclosed method involves proximity-mediated rolling circle amplification and real-time detection of amplification products where amplification includes multiply-primed rolling circle amplification (MPRCA). Rolling circle amplification (RCA) refers to nucleic acid amplification reactions involving replication of a circular nucleic acid template (referred to as an amplification target circle; ATC) to form a long strand (referred to as tandem sequence DNA; TS-DNA) with tandem repeats of the sequence complementary to the circular template. Rolling circle replication can be primed at one or more sites on the circular template. Multiply-primed RCA refers to RCA where replication is primed at a plurality of sites on the circular template. Multiply-primed RCA increases the sensitivity of singly-primed rolling circle amplification. Rolling circle amplification refers both to rolling circle replication and to processes involving both rolling circle replication and additional forms of amplification (such as replication of tandem sequence DNA).

Multiply-primed RCA can be performed using a single primer (which hybridizes to multiple sites on the amplification target circle) or multiple primers (each of which can hybridize to a single site on the amplification target circle or multiple sites on the amplification target circle). Multiple priming (as occurs in MPRCA) can increase the yield of amplified product from RCA. Primers anneal to multiple locations on the circular template and a product of extension by polymerase is initiated from each location. In this way, multiple extensions are achieved simultaneously from a single amplification target circle.

In some forms of the disclosed method, multiple priming can be achieved in several different ways. For example, two or more specific primers that anneal to different sequences on the circular template can be used, one or more specific primers that each anneals to a sequence repeated at two or more separate locations on the circular template can be used, a combination of primers that each anneal to a different sequence on the circular template or to a sequence repeated at two or more separate locations on the circular templates can be used, one or more random or degenerate primers, which can anneal to many locations on the circle, can be used, or a combination of such primers can be used.

Multiply-primed rolling circle amplification generates multiple tandem-sequence DNA (TS-DNA) copies from each circular template molecule. MPRCA can be used with circular template molecules of known, partially known, or unknown sequence, and the circular target DNA molecule can be single-stranded (ssDNA), double-stranded (dsDNA or duplex DNA), or partially double-stranded. Random or degenerate primers are useful for RCA of circular templates of unknown sequence.

Any or all of the primers used in the disclosed method can be resistant to degradation by exonuclease activity that may be present in the reaction. This has the advantage of permitting the primers to persist in reactions that contain an exonuclease activity and that may be carried out for long incubation periods. The persistence of primers allows new priming events to occur for the entire incubation time of the reaction, which is one of the hallmarks of exponential RCA (ERCA) and has the advantage of increasing the yield of amplified DNA.

Fluorescent change probes and primers, which are useful for obtaining real-time detection of amplification, refer to all probes and primers that involve a change in fluorescence intensity or wavelength based on a change in the form or conformation of the probe or primer and nucleic acid to be detected, assayed or replicated. Examples of fluorescent change probes and primers include molecular beacons, Amplifluors, FRET probes, cleavable FRET probes, TaqMan probes, scorpion primers, fluorescent triplex oligos including but not limited to triplex molecular beacons or triplex FRET probes, fluorescent water-soluble conjugated polymers, PNA probes and QPNA probes. Change in fluorescence wavelength or intensity from fluorescent change probes and primers generally involves energy transfer and/or quenching. Fluorescent change probes and primers can be classified according to their structure and/or function. Fluorescent change probes include, for example, hairpin quenched probes, cleavage quenched probes, cleavage activated probes, and fluorescent activated probes.

Random and/or degenerate probes and primers can be used with the disclosed method. As used herein, degenerate refers to an oligonucleotide (or oligomer) in which one or more of the base positions is occupied by more than one base, that is, a mixture of oligonucleotides (or oligomers) of defined length in which one or more positions of an individual member of the mixture is occupied by a base selected at random from among more than one possibilities for that position. Such collections of oligonucleotides (or oligomers) can be readily synthesized using standard oligonucleotide synthesis instruments and software. As used herein, random refers to an oligonucleotide (or oligomer) in which each of the base positions is occupied by a base selected at random from among a complete set of possibilities, but commonly limited to, for example, the four bases adenine (A), guanine (G), cytosine (C) and thymine (T) (or uracil (U)). For example, random oligonucleotides can be composed of the four nucleotides deoxyriboadenosine monophosphate (dAMP), deoxyribocytidine monophosphate (dCMP), deoxyriboguanosine monophosphate (dGMP), or deoxyribothymidine monophosphate (dTMP). Degenerate oligonucleotides (or oligomers) where not every base position is selected at random from among a complete set of possibilities can be referred to as partially random oligonucleotides (or oligomers). In some embodiments, the primers can contain nucleotides, including any types of modified nucleotides or nucleotide analogs, which can serve to make the primers resistant to enzyme degradation, to have other effects, or to give the primers useful properties.

The disclosed method can be used to amplify and detect any circular molecule. Circular template molecules to be subject to rolling circle replication and rolling circle amplification are referred to herein as amplification target circles (ATC). Amplification target circles can be, for example, designed and prefabricated for use in the disclosed method or can be produced from nucleic acid sources and samples of interest. For example, in some forms of the disclosed method, amplification target circles are designed and synthesized to have specific features making them useful for particular forms of the disclosed method. Such features are described in detail elsewhere herein. Amplification target circles can be two half circle probes that have been circularized. Such circularization is usefully accomplished via proximity-mediated ligation of the ends of one half circle probe to the ends of another half circle probe that is in close proximity. Amplification target circles can also be produced by circularizing nucleic acid molecules of interest or inserting nucleic acid molecules of interest into, for example, linker, vector or circularization sequences. Thus, target sequences can be copied or inserted into circular ssDNA or dsDNA by any suitable cloning or recombinant DNA technique. Amplification target circles can also be circular nucleic acid molecules isolated from cell, tissues or other nucleic acid samples. For example, plasmid DNA, viral DNA, and other circular nucleic acids can be used as amplification target circles in the disclosed method.

Genomic sequences can be amplified using the disclosed method. For example, known sequences or sequences of interest from genomic or other complex DNAs can be circularized or otherwise placed in amplification target circles for use in the disclosed method. Alternatively, amplification target circles generated in or from a whole genome amplification method can be used in the disclosed method. Whole genome amplification can involve randomly primed or specifically primed generation of all or a subset of genomic, cDNA or other complex DNA. Any suitable method then can be used to circularize the products of the whole genome amplification. The resulting amplification target circles could then be amplified in the disclosed method. Regardless of the means used to generate the circular products of whole genome amplification, multiply-primed RCA (using random primers, for example) would allow the selective amplification of the circles over any background of linear DNAs without the need for knowing the sequence of the circles. Alternatively, circular DNA containing known linker, vector, circularizing or target sequences would allow use of specific primer sequences for multiply-primed RCA.

Multiply-primed RCA represents an improvement over linear RCA (LRCA) in allowing increased rate of synthesis and increased yield. This results from the multiple priming sites for DNA polymerase extension. Use of random or degenerate primers also can have the benefit of generating double stranded products. This is because the linear ssDNA products generated by copying of the circular template will themselves be converted to duplex form by random (or degenerate) priming of DNA synthesis. Double stranded products can also be generated in most forms of DNA strand displacement replication, such as exponential RCA. Double stranded DNA product is advantageous in allowing for DNA sequencing of either strand and for restriction endonuclease digestion and other methods used in cloning, labeling, and detection.

It is to be understood that the disclosed method and compositions are not limited to specific synthetic methods, specific analytical techniques, or to particular reagents unless otherwise specified, and, as such, can vary. It is also to be understood that the terminology used herein is for the purpose of describing particular embodiments only and is not intended to be limiting.

### Materials

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a rolling circle replication primer is disclosed and discussed and a number of modifications that can be made to a number of molecules including the rolling circle replication primer are discussed, each and every combination and permutation of the rolling circle replication primer and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

A. Binding Guide Conjugates

A binding guide conjugate is, in some forms, a specific binding molecule coupled or tethered to a nucleic acid such as an oligonucleotide. The specific binding molecule is referred to as the affinity portion of the binding guide conjugate and the nucleic acid is referred to as the oligonucleotide portion of the binding guide conjugate. As used herein, a specific binding molecule is a molecule that interacts specifically with a particular molecule or moiety (that is, an analyte). The molecule or moiety that interacts specifically with a specific binding molecule is referred to herein as a target molecule. The target molecules can be any analyte. It is to be understood that the term target molecule refers to both separate molecules and to portions of molecules, such as an epitope of a protein, that interacts specifically with a specific binding molecule. Antibodies, either member of a receptor/ligand pair, and other molecules with specific binding affinities are examples of specific binding molecules, useful as the affinity portion of a binding guide conjugate. A binding guide conjugate with an affinity portion which is an antibody can be referred to herein as a binding guide antibody. The oligonucleotide portion can be a nucleic acid molecule or a combination of nucleic acid molecules. The oligonucleotide portion is preferably a guide oligonucleotide or a half circle probe.

By attaching a guide oligonucleotide to a specific binding molecule (to form a binding guide conjugate), binding of the specific binding molecule to its specific target can be detected. This can be accomplished by using two guide oligonucleotides (each part of a different binding guide conjugate) to mediate ligation of a pair of half circle probes to form an amplification target circle and amplifying the amplification target circle with rolling circle amplification. This amplification allows sensitive detection of a very small number of bound specific binding molecules. A binding guide conjugate that interacts specifically with a particular target molecule is said to be specific for that target molecule. For example, a binding guide conjugate with an affinity portion which is an antibody that binds to a particular antigen is said to be specific for that antigen. The antigen is the target molecule. Binding guide conjugates are also referred to herein as binding guide molecules.

One form of binding guide conjugate, which can be referred to as a binding guide analyte, is an analyte coupled or tethered to a nucleic acid such as an oligonucleotide. The analyte is referred to as the affinity portion of the binding guide analyte and the nucleic acid is referred to as the oligonucleotide portion of the binding guide analyte. The analyte in a binding guide analyte can be an analyte of interest or a binding mimetic of an analyte of interest. As used herein, a binding mimetic is a molecule or moiety that possesses the same or similar relevant binding characteristics of the analyte of interest. Relevant binding characteristics are those that are involved in the particular method, reagents or assay. An isolated epitope of an analyte is an example of a binding mimetic. It is to be understood that the term analyte refers to both separate molecules and to portions of molecules, such as an epitope of a protein. The oligonucleotide portion can be a nucleic acid molecule or a combination of nucleic acid molecules. The oligonucleotide portion is preferably a guide oligonucleotide or a half circle probe.

Preferred target molecules are proteins and peptides. Use of binding guide conjugates that target proteins and peptides allows sensitive signal amplification using rolling circle amplification for the detection of proteins and peptides. The ability to multiplex rolling circle amplification detection allows multiplex detection of the proteins and peptides (or any other target molecule). Thus, the disclosed method can be used for multi-protein analysis such as proteomics analysis. Such multi-protein analysis can be accomplished, for example, by using binding guide conjugate targeted to different proteins, with the oligonucleotide portion of each binding guide conjugate coded to allow separate amplification and detection of each different binding guide conjugate.

A binding guide conjugate can be said to correspond to a half circle probe if the guide oligonucleotide of the binding guide conjugate is complementary to a guide complement portion of the half circle probe. A binding guide conjugate can be said to correspond to another binding guide conjugate when one of the guide complement portions of a half circle probe is complementary to the guide oligonucleotide of the binding guide conjugate and the other guide complement portion of the half circle probe is complementary to the guide oligonucleotide of the other binding guide conjugate. Thus, binding guide conjugates that correspond to each other also correspond to the same half circle probe. Because the guide oligonucleotide of a binding guide conjugate can be complementary to two guide complement portions, each binding guide conjugate can correspond to two different half circle probes.

Two binding guide conjugates can be said to constitute a matched pair of binding guide conjugates when the two binding guide conjugates each correspond to the same two half circle probes. The two half circle probes to which the binding guide conjugates of a matched pair of binding guide conjugates correspond can be said to constitute a pair of half circle probes. The guide oligonucleotides of the binding guide conjugates in a matched pair of binding guide conjugates can be said to constitute a pair of guide oligonucleotides. In the disclosed method, each guide oligonucleotide in a pair of guide oligonucleotides can be complementary to one of the guide complement portions of each of the half circle probes in a pair of half circle probes and the other guide oligonucleotide of each guide oligonucleotide in the pair of guide oligonucleotides can be complementary to the other guide complement portion of each of the half circle probes in the pair of half circle probes. These relationships allow binding guide conjugates in a matched pair of binding guide conjugates that are in close proximity to mediate ligation of the half circle probes in a pair of half circle probes.

A matched pair of binding guide conjugates can also be referred to as a pair of binding guide conjugates. The term pair of binding guide conjugates can refer to both matched pairs of binding guide conjugates and other pairs of binding guide conjugates. As used herein, reference to a pair of binding guide conjugates refers to a matched pair of binding guide conjugates unless the context indicates otherwise. A pair of binding guide conjugates that is not a matched pair of binding guide conjugates can be referred to as an unmatched pair of binding guide conjugates.

In one embodiment, the oligonucleotide portion of a binding guide conjugate includes an oligonucleotide, referred to as a guide oligonucleotide, that provides guide sequences for half circle probes. The sequence of the guide sequences can be arbitrarily chosen. In a multiplex assay using multiple binding guide conjugates, it is preferred that the guide sequences for the guide oligonucleotide of each binding guide conjugate be substantially different to limit the possibility of non-specific target detection. Alternatively, it may be desirable in some multiplex assays, to use guide sequences with related sequences. By using different, unique gap oligonucleotides to fill different gap spaces, such assays can use one or a few pairs of half circle probes to label and detect a larger number of analytes. The oligonucleotide portion can be coupled to the affinity portion by any of several established coupling reactions. For example, Hendrickson et al., Nucleic Acids Res., 23(3):522-529 (1995) describes a suitable method for coupling oligonucleotides to antibodies.

In another embodiment, the oligonucleotide portion of a binding guide conjugate can include a half circle probe which, when ligated to another half circle probe, serves as a template for rolling circle replication. In a multiplex assay using multiple binding guide conjugate, it is preferred that primer complement portions, detection tag portions and/or whatever portions of the half circle probe comprising the oligonucleotide portion of each binding guide conjugate that match or are complementary to a fluorescent change probe or primer be substantially different to aid unique detection of each binding guide conjugate. Where fluorescent change probes are used, it is desirable to use the same primer complement portion in all of the half circle probes used in a multiplex assay. The half circle probe can be tethered to the specific binding molecule by looping the half circle probe around a tether loop and/or by hybridizing the half circle probe to a tether loop. This allows the amplification target circle that is formed when the half circle probe is ligated to another half circle probe to rotate freely during rolling circle replication while remaining coupled to the affinity portion. The tether loop can be any material that can form a loop and be coupled to a specific binding molecule. Linear polymers are a preferred material for tether loops.

A preferred method of producing a binding guide conjugate with a tethered half circle probe is to form the tether loop by ligating the ends of oligonucleotides coupled to a specific binding molecule around a half circle probe. Oligonucleotides can be coupled to specific binding molecules using known techniques. For example, Hendrickson et al. (1995), describes a suitable method for coupling oligonucleotides to antibodies. This method is generally useful for coupling oligonucleotides to any protein. To allow ligation, oligonucleotides comprising the two halves of the tether loop should be coupled to the specific binding molecule in opposite orientations such that the free end of one is the 5′ end and the free end of the other is the 3′ end. Ligation of the ends of the tether oligonucleotides can be mediated by hybridization of the ends of the tether oligonucleotides to adjacent sequences in the half circle probe to be tethered. In this way, the ends of the tether oligonucleotides are analogous to the target probe portions of an open circle probe, with the half circle probe containing the target sequence. Similar techniques can be used to form tether loops containing a target sequence.

The ends of tether loops can be coupled to any specific binding molecule with functional groups that can be derivatized with suitable activating groups. When the specific binding molecule is a protein, or a molecule with similar functional groups, coupling of tether ends can be accomplished using known methods of protein attachment. Many such methods are described in Protein immobilization: fundamentals and applications Richard F. Taylor, ed. (M. Dekker, New York, 1991).

The half circle probe can also be hybridized to a tether. Such an attachment mode can keep the half circle probe in place until ligation to another half circle probe while allowing the resulting amplification target circle to be released for rolling circle amplification. The half circle probe can also be attached to the specific binding molecule in a binding guide conjugate via a cleavable linkage or bond. After the half circle probe is ligated to another half circle probe to form an amplification target circle, the amplification target circle can be released for rolling circle amplification by breaking the cleavable linkage or bond. As used herein, a cleavable linkage or bond refers to any linkage or bond that can be specifically broken or disrupted. Such linkage and bond breakage can include, for example, enzymatic cleavage, chemical cleavage, or dissociation of the bond by solution conditions such as ionic strength, pH, or the presence of reducing agents.

The oligonucleotide portion of a binding guide conjugate can also be a guide oligonucleotide having both ends coupled to the specific binding molecule so as to form a loop. In this way, when the half circle probes hybridize to the guide oligonucleotide and are circularized, the half circle probes will remain topologically locked to the binding guide conjugate during rolling circle replication of the circularized half circle probes. This improves the localization of the resulting amplified signal to the location where the binding guide conjugate is bound (that is, at the location of the target molecule).

A special form of binding guide molecule, referred to herein as a binding guide probe, has an oligonucleotide or oligonucleotide derivative as the specific binding molecule. Binding guide probes are designed for and used to detect specific nucleic acid sequences. Thus, the target molecule for binding guide probes are nucleic acid sequences. The target molecule for a binding guide probe can be a nucleotide sequence within a larger nucleic acid molecule. It is to be understood that the terms binding guide molecule and binding guide conjugate encompass binding guide probes. The specific binding molecule of a binding guide probe can be any length that supports specific and stable hybridization between the binding guide probe and the target molecule. For this purpose, a length of 10 to 40 nucleotides is preferred, with a specific binding molecule of a binding guide probe 16 to 25 nucleotides long being most preferred.

It is preferred that the specific binding molecule of a binding guide probe is peptide nucleic acid. Peptide nucleic acid forms a stable hybrid with DNA. This allows a binding guide probe with a peptide nucleic acid specific binding molecule to remain firmly adhered to the target sequence during subsequent amplification and detection operations. This useful effect can also be obtained with binding guide probes with oligonucleotide specific binding molecules by making use of the triple helix chemical bonding technology described by Gasparro et al., Nucleic Acids Res. 1994 22(14):2845-2852 (1994). Briefly, the affinity portion of a binding guide probe is designed to form a triple helix when hybridized to a target sequence. This is accomplished generally as known, preferably by selecting either a primarily homopurine or primarily homopyrimidine target sequence. The matching oligonucleotide sequence which constitutes the affinity portion of the binding guide probe will be complementary to the selected target sequence and thus be primarily homopyrimidine or primarily homopurine, respectively. The binding guide probe (corresponding to the triple helix probe described by Gasparro et al.) contains a chemically linked psoralen derivative. Upon hybridization of the binding guide probe to a target sequence, a triple helix forms. By exposing the triple helix to low wavelength ultraviolet radiation, the psoralen derivative mediates cross-linking of the probe to the target sequence.

The specific binding molecule in a binding guide probe can also be a bipartite DNA molecule, such as ligatable DNA probes adapted from those described by Landegren et al., Science 241:1077-1080 (1988). When using such a probe, the affinity portion of the probe is assembled by target-mediated ligation of two oligonucleotide portions which hybridize to adjacent regions of a target nucleic acid. Thus, the components used to form the affinity portion of such binding guide probes are a truncated binding guide probe (with a truncated affinity portion which hybridizes to part of the target sequence) and a ligation probe which hybridizes to an adjacent part of the target sequence such that it can be ligated to the truncated binding guide probe. The ligation probe can also be separated from (that is, not adjacent to) the truncated binding guide probe when both are hybridized to the target sequence. The resulting space between them can then be filled by a second ligation probe or by gap-filling synthesis. For use in the disclosed methods, it is preferred that the truncated affinity portion be long enough to allow target-mediated ligation but short enough to, in the absence of ligation to the ligation probe, prevent stable hybridization of the truncated binding guide probe to the target sequence during the subsequent amplification operation. For this purpose, a specific step designed to eliminate hybrids between the target sequence and unligated truncated binding guide probes can be used following the ligation operation.

In another embodiment, the guide oligonucleotide of a binding guide conjugate can serve as a rolling circle replication primer. This allows rolling circle replication of the ATC formed by ligation of half circle probes where the resulting TS-DNA is coupled to the binding guide conjugate. Because of this, the TS-DNA will be effectively immobilized at the site of the target molecule. Preferably, the immobilized TS-DNA can then be collapsed in situ prior to detection. The sequence of the rolling circle replication primer sequence can be arbitrarily chosen. Additional, untethered rolling circle replication primers can also be used to achieve multiply-primed RCA.

In a multiplex assay using multiple binding guide conjugates, it is preferred that the fluorescent change probes or primers used with each binding guide conjugate be substantially different to limit the possibility of non-specific target detection. Alternatively, it may be desirable in some multiplex assays, to use fluorescent change probes or primers with related sequences. Such assays can use one or a few ATCs to detect a larger number of target molecules. Any of the other relationships between ATCs and primers and probes disclosed herein can also be used. When the oligonucleotide portion of a binding guide conjugate is used as a rolling circle replication primer, the oligonucleotide portion can be any length that supports specific and stable hybridization between the oligonucleotide portion and the primer complement portion of an amplification target circle. Generally this is 10 to 35 nucleotides long, but is preferably 16 to 20 nucleotides long.

Antibodies useful as the affinity portion of binding guide conjugates, can be obtained commercially or produced using well established methods. For example, Johnstone and Thorpe, Immunochemistry In Practice (Blackwell Scientific Publications, Oxford, England, 1987) pages 30-85, describe general methods useful for producing both polyclonal and monoclonal antibodies. The entire book describes many general techniques and principles for the use of antibodies in assay systems.

B. Guide Oligonucleotides

A guide oligonucleotide is an oligonucleotide or oligomer having sequences complementary to guide complement portions of half circle probes. These sequences are referred to as guide sequences. The guide sequences of a guide oligonucleotide and the cognate guide complement portion can have any desired sequence so long as they are complementary to each other. In general, the sequence of the guide oligonucleotide can be chosen such that it is not significantly complementary to any other portion of the half circle probes. That is, the guide oligonucleotide would be complementary only to guide complement portions. The guide sequences of a guide oligonucleotide can be any length that supports specific and stable hybridization between the guide oligonucleotide and the guide complement portion. Generally this is 10 to 35 nucleotides long, but is preferably 16 to 20 nucleotides long.

Guide oligonucleotides can have sequences (or other structures) other than guide sequences. For example, guide oligonucleotides can have a gap sequence between guide sequences. Guide oligonucleotides that are part of a binding guide conjugate can have, for example, a spacer or linker beyond the guide sequences that is used to attach the guide oligonucleotide to the specific binding molecule in the binding guide conjugate. Guide oligonucleotides can be made up of or include modified nucleotides and nucleotide analogs. In some forms of the disclosed method, guide oligonucleotides are attached or coupled to specific binding molecules to form binding guide conjugates. In other forms of the disclosed method, guide oligonucleotides are not attached or coupled to another molecule or component.

In some forms of the method, the guide oligonucleotides can contain a spacer. The spacer can help to overcome steric factors from the surface when attached to binding guide conjugates. Spacers useful for the disclosed method include nucleotide spacers such as poly dT or poly dA; aliphatic linkers such as C18, C12, or multimers thereof; aromatic spacers, or RNA, DNA, PNA or combinations thereof.

Guide oligonucleotides typically have two guide sequences, but can have more than two guide sequences. The two guide sequences in a guide oligonucleotide can be complementary to guide complement portions in two different half circle probes. That is, one of the guide sequences in a guide oligonucleotide can be complementary to a guide complement portion of one half circle probe and the other guide sequence in the guide oligonucleotide can be complementary to a guide complement portion of another half circle probe.

Guide oligonucleotides can have one free end or no free ends. A free end is not required because a free end is not required for hybridization of half circle probes. The free end of a guide oligonucleotide can be a 5′ end or a 3′ end, although a 5′ end is preferred to reduce the possibility that the guide oligonucleotide would interfere with rolling circle replication. Alternatively, the guide oligonucleotide can have a 3′ end. This is useful if the guide oligonucleotide is to serve as a rolling circle replication primer (or other primer). A guide oligonucleotide can also have two free ends if it is coupled or attached to a specific binding molecule or analyte at an internal (i.e. non-end) location.

A guide oligonucleotide can be said to correspond to a half circle probe if the guide oligonucleotide is complementary to a guide complement portion of the half circle probe. A guide oligonucleotide can be said to correspond to another guide oligonucleotide when one of the guide complement portions of a half circle probe is complementary to the guide oligonucleotide and the other guide complement portion of the half circle probe is complementary to the other guide oligonucleotide. Thus, guide oligonucleotides that correspond to each other also correspond to the same half circle probe. Because a guide oligonucleotide can be complementary to two guide complement portions, each guide oligonucleotide can correspond to two different half circle probes.

Two guide oligonucleotides can be said to constitute a matched pair of guide oligonucleotides when the two guide oligonucleotides each correspond to the same two half circle probes. The two half circle probes to which the guide oligonucleotides of a matched pair of guide oligonucleotides correspond can be said to constitute a pair of half circle probes. The binding guide conjugates of which the guide oligonucleotides in a matched pair of guide oligonucleotides are a part can be said to constitute a pair of binding guide conjugates. In the disclosed method, each guide oligonucleotide in a matched pair of guide oligonucleotides can be complementary to one of the guide complement portions of each of the half circle probes in a pair of half circle probes and the other guide oligonucleotide of each guide oligonucleotide in the matched pair of guide oligonucleotides can be complementary to the other guide complement portion of each of the half circle probes in the pair of half circle probes. These relationships allow binding guide conjugates that are in close proximity to mediate ligation of the half circle probes in a pair of half circle probes.

A matched pair of guide oligonucleotides can also be referred to as a pair of guide oligonucleotides. The term pair of guide oligonucleotides can refer to both matched pairs of guide oligonucleotides and other pairs of guide oligonucleotides. As used herein, reference to a pair of guide oligonucleotides refers to a matched pair of guide oligonucleotides unless the context indicates otherwise. A pair of guide oligonucleotides that is not a matched pair of guide oligonucleotides can be referred to as an unmatched pair of guide oligonucleotides.

C. Half Circle Probes

A half circle probe (HCP) is a linear DNA molecule. HCPs can be any length, but preferably contain between 25 to 500 nucleotides, more preferably between about 30 to 75 nucleotides, and most preferably between about 35 to 50 nucleotides. The HCP has a 5′ phosphate group and a 3′ hydroxyl group. This allows the ends to be ligated (to another HCP or to other nucleic acid ends) using a ligase, coupled, or extended in a gap-filling operation. Half circle probes can be partially double-stranded.

One end of a half circle probe can be ligated or coupled to one end of another half circle probe to form an open circle probe. Also, both ends of a half circle probe can be ligated or coupled to both ends of another half circle probe to form an amplification target circle. A pair of half circle probes that are coupled or ligated together to form either an open circle probe or amplification target circle can be referred to as corresponding half circle probes. Corresponding half open probes need not be identical. Preferably, in a pair of HCPs, each HCP is different. For example, the HCPs can have a different sequences and/or lengths than one another. Useful half circle probes can comprise one or more primer complement portions, one or more secondary DNA strand displacement primer matching portions, and one or more detection tag portions.

A half circle probe can be said to correspond to a guide oligonucleotide if a guide complement portion of the half circle probe is complementary to the guide oligonucleotide. A half circle probe can be said to correspond to another half circle probe when a guide complement portion of the half circle probe is complementary to the same guide oligonucleotide as a guide complement portion of the other half circle probe such that the guide complement portions can be ligated together (including via gap oligonucleotides and/or gap-filling synthesis) when hybridized to the guide oligonucleotide. Thus, half circle probes that correspond to each other also correspond to the same guide oligonucleotide.

Two half circle probes can be said to constitute a matched pair of half circle probes when the two half circle probes each correspond to the same two guide oligonucleotides. The two guide oligonucleotides to which the half circle probes of a matched pair of half circle probes correspond can be said to constitute a pair of guide oligonucleotides. The binding guide conjugates that the guide oligonucleotides in a pair of guide oligonucleotides are a part of can be said to constitute a pair of binding guide conjugates. In the disclosed method, one guide complement portion of each half circle probe in a matched pair of half circle probes can be complementary to one of the guide oligonucleotides in a pair of guide oligonucleotides and the other guide complement portion of each half circle probe in the matched pair of half circle probes can be complementary to the other guide oligonucleotide in the pair of guide oligonucleotides. These relationships allow binding guide conjugates that are in close proximity to mediate ligation of the half circle probes in a matched pair of half circle probes.

A matched pair of half circle probes can also be referred to as a pair of half circle probes. The term pair of half circle probes can refer to both matched pairs of half circle probes and other pairs of half circle probes. As used herein, reference to a pair of half circle probes refers to a matched pair of half circle probes unless the context indicates otherwise. A pair of half circle probes that is not a matched pair of half circle probes can be referred to as an unmatched pair of half circle probes.

Portions of a HCP can have specific functions making the HCP useful for RCA, pmRCA, and LM-RCA. These portions are referred to as the guide complement portions, the primer complement portions, the spacer region, the secondary DNA strand displacement primer matching portions, the detection tag portions, the secondary target sequence portions, the address tag portions, and the promoter portions. These portions are analogous to similarly-named portions of open circle probes and amplification target circles and their further description elsewhere herein in the context of open circle probes and amplification target circles is applicable to the analogous portion in HCPs. The spacer region of a half circle probe refers to sequences not part of guide complement portions.

The primer complement portions, additional secondary target sequence portions, detection tag portions, address tag portions, and promoter portions can all be on one of the HCPs in a pair of corresponding HCPs. Some of these portions can be on one HCP and other portions can be on the other HCP in a pair of corresponding HCPs. For example, one half circle probe can have primer complement portions and additional secondary target sequence portions and the other half circle probe can have detection tag portions, address tag portions, and promoter portions. Both HCPs in a pair of corresponding HCPs can have primer complement portions, additional secondary target sequence portions, detection tag portions, address tag portions, and promoter portions.

The guide complement portions are required elements of a half circle probe. At least one primer complement portion must be present on one of the half circle probes in a pair of half circle probes. The primer complement portion can be part of, for example, the spacer region of one of the half circle probes in the pair of half circle probes. Detection tag portions, secondary target sequence portions, promoter portions, and additional primer complement portions are optional and, when present, can be part of, for example, the spacer region of one of the half circle probes or both of the half circle probes in a pair of half circle probes. Address tag portions are optional and, when present, can be part of, for example, the spacer region of one of the half circle probes or both of the half circle probes in a pair of half circle probes. The primer complement portions, and the detection tag portions, the secondary target sequence portions, the address tag portions, and the promoter portions, if present, can be non-overlapping. However, various of these portions can be partially or completely overlapping if desired. HCPs can be single-stranded but may be partially double-stranded. In use, the guide complement portions of a HCP should be single-stranded so that they can interact with target sequences, such as the guide oligonucleotides.

Generally, a half circle probe can be a single-stranded, linear DNA molecule comprising, from 5′ end to 3′ end, a 5′ phosphate group, a guide complement portion, a spacer region, a guide complement portion, and a 3′ hydroxyl group, with a primer complement portion present as part of the spacer region. Particularly useful half circle probes can comprise two guide complement portions, one or more primer complement portions, and a secondary DNA strand displacement primer matching portion. Those segments of the spacer region that do not correspond to a specific portion of the HCP can be arbitrarily chosen sequences. For multiply-primed RCA, a plurality of primer complement portions are required. Where random or degenerate rolling circle replication primers are used, the sequence of the primer complement portions need not either be known or of a specified sequence. The half circle probe can include at least one detection tag portion when fluorescent change probes (or other detection probes) are used for detection.

It is preferred that HCPs do not have any sequences that are self-complementary. It is considered that this condition is met if there are no complementary regions greater than six nucleotides long without a mismatch or gap. It is also preferred that HCPs containing a promoter portion do not have any sequences that resemble a transcription terminator, such as a run of eight or more thymidine nucleotides. A lack of self-complementary sequences and a lack of promoter sequences is generally not required in the case of half circle probes including, derived from, or comprising nucleic acid molecules of interest. Such features will generally not be controlled for such half circle probes.

Half circle probes, when ligated together and replicated, give rise to a long DNA molecule containing multiple repeats of sequences complementary to both half circle probes. This long DNA molecule is referred to herein as tandem sequence DNA (TS-DNA). TS-DNA contains sequences complementary to the guide complement portions, the primer complement portion, the spacer region, and, if present on one or both of the circularized half circle probes, the detection tag portions, the secondary target sequence portions, the address tag portions, and the promoter portions. These sequences in the TS-DNA are referred to as guide sequences (which match the original guide sequences in the guide oligonucleotides), primer sequences (which match the sequence of the rolling circle replication primer), spacer sequences (complementary to the spacer region), detection tags, secondary target sequences, address tags, and promoter sequences. The TS-DNA will also have sequence complementary to the matching portion of secondary DNA strand displacement primers. This sequence in the TS-DNA is referred to as the secondary DNA strand displacement primer complement or as the primer complement.

Preferably, the promoter portion of a HCP is immediately adjacent to a guide complement portion and is oriented to promote transcription toward the 3′ end of the half circle probe. This orientation results in transcripts that are complementary to TS-DNA, allowing independent detection of TS-DNA and the transcripts, and prevents transcription from interfering with rolling circle replication.

Half circle probes can be capable of forming an intramolecular stem structure involving one or both of the HCP's ends. Such half circle probes are referred to herein as hairpin half circle probes. Open circle probes forming intramolecular stem structures, and their use in rolling circle amplification, are described in U.S. patent application Ser. No. 09/803,713. These same principles can be applied and used with half circle probes forming intramolecular stem structures. Half circle probes including, derived from, or comprising nucleic acid molecules of interest can be any useful size, including, for example, the size of a plasmid, virus, vector, or artificial chromosome.

1. Guide Complement Portions

There are two guide complement portions on each HCP, one at each end of the HCP. A half circle probe can be said to be bi-specific when one guide complement portion is complementary to one guide oligonucleotide and the other guide complement portion is complementary to a different guide oligonucleotide. The guide complement portions can each be any length that supports specific and stable hybridization between the guide complement potion and the guide oligonucleotide. For this purpose, a length of 10 to 35 nucleotides for each guide complement portion is preferred, with guide complement portions 15 to 25 nucleotides long being most preferred. The guide complement portion at the 3′ end of the HCP is referred to as the 3′ guide complement portion, and the guide complement portion at the 5′ end of the HCP is referred to as the 5′ guide complement portion. These guide complement portions are also referred to herein as 3′ and 5′ guide complements. The guide complement portions are complementary to guide sequence.

The guide complement portions are complementary to the guide oligonucleotide sequences of binding guide conjugates, such that upon hybridization the 5′ guide complement portion of a first HCP and the 3′ guide complement portion of a second HCP are base-paired to adjacent nucleotides in a guide oligonucleotide sequence of one binding guide conjugate with the objective that they serve as a substrate for ligation. The 5′ guide complement portion of the second HCP and the 3′ guide complement portion of the first HCP also can base pair to adjacent nucleotides in a guide oligonucleotide sequence of a second binding guide conjugate, with the objective that they serve as a substrate for ligation. Ligation of both ends of both HCPs can be facilitated when the two binding guide conjugates are in close proximity.

In another form, the 5′ guide complement one HCP and the 3′ guide complement of the another HCP may hybridize to a guide oligonucleotide in such a way that they are separated by a gap space. In this case the ends of the HCPs may only be ligated if one or more additional oligonucleotides, referred to as gap oligonucleotides, are used, or if the gap space is filled during the ligation operation. The gap oligonucleotides hybridize to the gap sequence in the guide oligonucleotide (opposite the gap space) to form a continuous probe/target hybrid. The gap space may be any length desired but is generally ten nucleotides or less. It is preferred that the gap space is between about three to ten nucleotides in length, with a gap space of four to eight nucleotides in length being most preferred. Alternatively, a gap space could be filled using a DNA polymerase during the ligation operation. When using such a gap-filling operation, a gap space of three to five nucleotides in length is most preferred. As another alternative, the gap space can be partially bridged by one or more gap oligonucleotides, with the remainder of the gap filled using DNA polymerase.

When two half circle probes are ligated to one another at one end, the two guide complement portions at the unligated ends of the half circle probes can act as target probe portions in an open circle probe. In this way, the guide complement portion of a half circle probe can be analogous to the target probe portion of an open circle probe.

2. Primer Complement Portions

Primer complement portions are parts of a half circle probe that are complementary to rolling circle replication primers (RCRP). Each HCP or pair of HCPs preferably has multiple primer complement portions. This allows rolling circle replication to initiate at multiple sites on the amplification target circle produced from the half circle probes. However, a HCP or pair of HCPs can include one or more than one primer complement portion. If multiple primer complement portions are present, they can have sequence complementary to the same rolling circle replication primer, different rolling circle replication primers, or a combination of the same and different rolling circle replication primers. A primer complement portion and its cognate primer can have any desired sequence so long as they are complementary to each other. The sequence of the primer complement portion is referred to as the primer complement sequence.

In general, the sequence of a primer complement portion can be chosen such that it is not significantly similar to any other portion of the half circle probe. The sequence of the primer complement portion also can be chosen such that it is not significantly similar to any portion of a half circle probe in a pair with the half circle probe having the primer complement portion. The primer complement portion can be any length that supports specific and stable hybridization between the primer complement portion and the primer. For this purpose, a length of 10 to 35 nucleotides is preferred, with a primer complement portion 16 to 20 nucleotides long being most preferred. If random or degenerate rolling circle replication primers are used, the half circle probes will have multiple primer complement portions that generally will not be, and need not be, specifically identified. If random or degenerate rolling circle replication primers are used, the primers and the primer complement portions are preferably 4 to 10 nucleotides long, and most preferably 6, 7 or 8 nucleotides long.

The primer complement portions can be located anywhere on a HCP, such as within the spacer region of a HCP. Primer complement portions can be anywhere on a HCP or pair of HCPs, pair of HCPs. For example, the primer complement portions can be adjacent to a guide complement portion of a half circle probe. For example, the primer complement portion and the 5′ guide complement portion can be separated by three to ten nucleotides, and preferably separated by six nucleotides. This location prevents the generation of any other spacer sequences, such as detection tags and secondary target sequences, from unligated half circle probes during DNA replication. Such an arrangement is less useful when using multiply-primed RCA. However, both half circle probes in a pair of half circle probes can have, for example, a primer complement portion proximate to the 5′ guide complement portion. A primer complement portion can also be a part of or overlap all or a part of the guide complement portions and/or any gap space sequence, if present.

3. Secondary DNA Strand Displacement Primer Matching Portions

Secondary DNA strand displacement primer matching portions are parts of a half circle probe that match sequence in secondary DNA strand displacement primers. The sequence in a secondary DNA strand displacement primer that matches a secondary DNA strand displacement primer matching portion in a HCP or pair of HCPs is referred to as the matching portion of the secondary DNA strand displacement primer. A HCP or a pair of HCPs can include one or more than one primer matching portion. If multiple primer matching portions are present, they can have sequence matching the same secondary DNA strand displacement primer (which is preferred), different secondary DNA strand displacement primers, or a combination of the same and different secondary DNA strand displacement primers. A single secondary DNA strand displacement primer matching portion is preferred. A primer matching portion and its cognate primer can have any desired sequence so long as they are complementary to each other. The sequence of the primer matching portion can be referred to as the primer matching sequence. More specifically, the sequence of the secondary DNA strand displacement primer matching portion can be referred to as the secondary DNA strand displacement primer matching sequence.

In general, the sequence of a primer matching portion can be chosen such that it is not significantly similar to any other portion of the HCP. The sequence of the primer matching portion also can be chosen such that is it not significantly similar to any portion of a half circle probe in a pair with the half circle probe having the primer matching portion. Primer matching portions can overlap with primer complement portions, although it is preferred that they not overlap. The primer matching portion can be any length that supports specific and stable hybridization between the primer complement portion in the resulting TS-DNA and the primer. For this purpose, a length of 10 to 35 nucleotides is preferred, with a primer matching portion 16 to 20 nucleotides long being most preferred. The primer matching portion can be located anywhere on a HCP, such as within the spacer region of a HCP. Primer matching portions can be anywhere on a HCP or pair of HCPs. If random or degenerate rolling circle replication primers are used, they can act as secondary DNA strand displacement primer. In this case, the half circle probes will have multiple secondary DNA strand displacement primer matching portions that generally will not be, and need not be, specifically identified. If random or degenerate rolling circle replication primers are used, the primers and the secondary DNA strand displacement primer matching portions are preferably 4 to 10 nucleotides long, and most preferably 6, 7 or 8 nucleotides long.

4. Detection Tag Portions

Detection tag portions are parts of a half circle probe that have sequence matching the sequence of the complementary portion of detection probes. These detection tag portions, when amplified during rolling circle replication, result in TS-DNA having detection tag sequences that are complementary to the complementary portion of detection probes. If present, there can be one, two, three, or more than three detection tag portions on a HCP or pair of HCPs. For example, a HCP or pair of HCPs can have two, three or four detection tag portions. Most preferably, a HCP or pair of HCPs will have three detection tag portions. Generally, it is preferred that a HCP or pair of HCPs have 60 detection tag portions or less. There is no fundamental limit to the number of detection tag portions that can be present on a HCP or pair of HCPs except the size of the HCPs. When there are multiple detection tag portions, they can have the same sequence or they can have different sequences, with each different sequence complementary to a different detection probe. It is preferred that a HCP or pair of HCPs contain detection tag portions that have the same sequence such that they are all complementary to a single detection probe. For some multiplex detection methods, it is preferable that HCPs or pair of HCPs contain up to six detection tag portions and that the detection tag portions have different sequences such that each of the detection tag portions is complementary to a different detection probe. If the half circle probes include, are derived from, or comprise nucleic acid molecules of interest, some or all of the detection tag portions can be sequences of interest in the nucleic acid of interest. In this way, detection can be based on the amplification of the specific sequences of interest. The detection tag portions can each be any length that supports specific and stable hybridization between the detection tags and the detection probe. For this purpose, a length of 10 to 35 nucleotides is preferred, with a detection tag portion 15 to 20 nucleotides long being most preferred.

5. Secondary Target Sequence Portions

Secondary target sequence portions are parts of a half circle probe that have sequence matching the sequence of target probes of a secondary amplification target circle or open circle probe. These secondary target sequence portions, when amplified during rolling circle replication, result in TS-DNA having secondary target sequences that are complementary to target probes of a secondary open circle probe. If present, there can be one, two, or more than two secondary target sequence portions on a HCP or pair of HCPs. It is preferred that a HCP or pair of HCPs have one or two secondary target sequence portions. Most preferably, a HCP or pair of HCPs will have one secondary target sequence portion. Generally, it is preferred that a HCP or pair of HCPs have 50 secondary target sequence portions or less. There is no fundamental limit to the number of secondary target sequence portions that can be present on a HCP or pair of HCPs except the size of the HCPs. When there are multiple secondary target sequence portions, they can have the same sequence or they can have different sequences, with each different sequence complementary to a different secondary open circle probe. It is preferred that a HCP or pair of HCPs contain secondary target sequence portions that have the same sequence such that they are all complementary to a single target probe portion of a secondary open circle probe. If the half circle probes include, are derived from, or comprise nucleic acid molecules of interest, some or all of the secondary target sequence portions can be sequences of interest in the nucleic acid of interest. In this way, further amplification can be based on the presence of the specific sequences of interest.

The secondary target sequence portions can each be any length that supports specific and stable hybridization between the secondary target sequence and the target sequence probes of its cognate secondary open circle probe. For this purpose, a length of 20 to 70 nucleotides is preferred, with a secondary target sequence portion 30 to 40 nucleotides long being most preferred. As used herein, a secondary open circle probe is an open circle probe where the target probe portions match or are complementary to secondary target sequences in another open circle probe, half circle probe, pair of half circle probes, or amplification target circle. It is contemplated that a secondary open circle probe can itself contain secondary target sequences that match or are complementary to the target probe portions of another secondary open circle probe. Secondary open circle probes related to each other in this manner are referred to herein as nested open circle probes.

6. Address Tag Portions

Address tag portions are parts of a half circle probe that have sequence matching the sequence of the complementary portion of an address probe. This address tag portion, when amplified during rolling circle replication, results in TS-DNA having address tag sequences that are complementary to the complementary portion of address probes. If present, there can be one, or more than one, address tag portion on a HCP or pair of HCPs. It is preferred that a HCP or pair of HCPs have one or two address tag portions. Most preferably, a HCP or pair of HCPs will have one address tag portion. Generally, it is preferred that a HCP or pair of HCPs have 50 address tag portions or less. There is no fundamental limit to the number of address tag portions that can be present on a HCP or pair of HCPs except the size of the HCPs. When there are multiple address tag portions, they can have the same sequence or they can have different sequences, with each different sequence complementary to a different address probe. It is preferred that a HCP or pair of HCPs contain address tag portions that have the same sequence such that they are all complementary to a single address probe. The address tag portion can be any length that supports specific and stable hybridization between the address tag and the address probe. For this purpose, a length between 10 and 35 nucleotides long is preferred, with an address tag portion 15 to 20 nucleotides long being most preferred. The address tag portion can be part of the guide complement portions, the spacer region, or both. The address tag portion preferably overlaps all or a portion of the guide complement portions of two half circle probes, and all of any intervening gap space. Most preferably, the address tag portion overlaps all or a portion of the 5′ guide complement portion of one half circle probe in a pair of half circle probes and the 3′ guide complement portion of the other half circle probe in the pair of half circle probes.

7. Promoter Portions

The promoter portion corresponds to the sequence of an RNA polymerase promoter. A promoter portion can be included in a half circle probe circle probe so that transcripts can be generated from the resulting ATC or TS-DNA. The sequence of any promoter can be used, but simple promoters for RNA polymerases without complex requirements are preferred. It is also preferred that the promoter is not recognized by any RNA polymerase that may be present in the sample containing the target nucleic acid sequence. Preferably, the promoter portion corresponds to the sequence of a T7 or SP6 RNA polymerase promoter. The T7 and SP6 RNA polymerases are highly specific for particular promoter sequences. Other promoter sequences specific for RNA polymerases with this characteristic would also be preferred. Because promoter sequences are generally recognized by specific RNA polymerases, the cognate polymerase for the promoter portion of a HCP or pair of HCPs should be used for transcriptional amplification. Numerous promoter sequences are known and any promoter specific for a suitable RNA polymerase can be used. The promoter portion can be located anywhere within a HCP or pair of HCPs and can be in either orientation.

D. Amplification Target Circles

An amplification target circle (ATC) is a circular DNA molecule. ATCs are preferably single-stranded but can be partially or fully double-stranded. Portions of ATCs can have specific functions making the ATC useful for rolling circle amplification (RCA). These portions are referred to as the primer complement portions, the secondary DNA strand displacement primer matching portions, the detection tag portions, the secondary target sequence portions, the address tag portions, and the promoter portions. These portions are analogous to similarly-named portions of half circle probes and open circle probes and their further description elsewhere herein in the context of half circle probes and open circle probes is applicable to the analogous portion in amplification target circles. At least one primer complement portion is a required element of an amplification target circle. For multiply-primed RCA, a plurality of primer complement portions are required. Where random or degenerate rolling circle replication primers are used, the sequence of the primer complement portions need not either be known or of a specified sequence. The amplification target circle can include at least one detection tag portion when fluorescent change probes (or other detection probes) are used for detection. Amplification target circles can include a spacer region. The spacer region of an amplification target circle refers to sequences not part of guide complement portions or target probe portions.

Secondary DNA strand displacement primer matching portions, detection tag portions, secondary target sequence portions, address tag portions, and promoter portions are optional. The primer complement portions, the secondary DNA strand displacement primer matching portions, the detection tag portions, the secondary target sequence portions, the address tag portions, and the promoter portion, if present, are preferably non-overlapping. However, various of these portions can be partially or completely overlapping if desired. Generally, an amplification target circle can be a circular DNA molecule comprising one or more primer complement portions. Amplification target circles can be single-stranded, double-stranded, or partially double-stranded. Useful amplification target circles can comprise one or more primer complement portions, one or more secondary DNA strand displacement primer matching portions, and one or more detection tag portions.

Those segments of the ATC that do not correspond to a specific portion of the ATC can be arbitrarily chosen sequences. It is preferred that ATCs do not have any sequences that are self-complementary, although this is not required. It is considered that this condition is met if there are no complementary regions greater than six nucleotides long without a mismatch or gap. It is also preferred that ATCs containing a promoter portion do not have any sequences that resemble a transcription terminator, such as a run of eight or more thymidine nucleotides. A lack of self-complementary sequences and a lack of promoter sequences is generally not required in the case of amplification target circles including, derived from, or comprising nucleic acid molecules of interest. Such features will generally not be controlled for such amplification target circles.

Two half circle probes that are ligated and circularized together are a type of ATC. Also, ligated and circularized open circle probes are a type of ATC. As used herein the term amplification target circle includes ligated open circle probes and circularized open circle probes as well as ligated pairs of half circle probes and circularized pairs of half circle probes. Amplification target circles can also be formed from the ligation and circularization of more than two half circle probes. An ATC can be used in the same manner as described herein for open circle probes and a pair of HCPs that have been ligated or circularized. Amplification target circles can be any desired length. Generally, amplification target circles designed for use as amplifiable labels can contain between 40 to 1000 nucleotides, more preferably between about 50 to 150 nucleotides, and most preferably between about 50 to 100 nucleotides. Amplification target circles including, derived from, or comprising nucleic acid molecules of interest can be any useful size, including, for example, the size of a plasmid, virus, vector, or artificial chromosome.

An amplification target circle, when replicated, gives rise to a long DNA molecule containing multiple repeats of sequences complementary to the amplification target circle. This long DNA molecule is referred to herein as tandem sequence DNA (TS-DNA). TS-DNA contains sequences complementary to the primer complement portions and, if present on the amplification target circle, the secondary DNA strand displacement primer matching portions, the detection tag portions, the secondary target sequence portions, the address tag portions, and the promoter portions. These sequences in the TS-DNA are referred to as primer sequences (which match the sequence of the rolling circle replication primers), spacer sequences (complementary to the spacer region), detection tags, secondary target sequences, address tags, and promoter sequences. The TS-DNA will also have sequence complementary to the matching portion of secondary DNA strand displacement primers. This sequence in the TS-DNA is referred to as the secondary DNA strand displacement primer complement or as the primer complement. Amplification target circles are useful as tags for specific binding molecules.

Where the ATC is formed from an open circle probe, the address tag portion can be part of the target probe portions, the spacer region, or both. Where the ATC is formed from a pair of half circle probes, the address tag portion can be part of the guide complement portions, the spacer region, or both. In these cases, the address tag portion preferably overlaps all or a portion of the target probe portions or guide complement portions, and all of any intervening gap space. Most preferably, the address tag portion overlaps all or a portion of both the left and right target probe portions of an open circle probe or the 5′ guide complement portion of one half circle probe in a pair of half circle probes and the 3′ guide complement portion of the other half circle probe in the pair of half circle probes.

E. Rolling Circle Replication Primers

A rolling circle replication primer (RCRP) is an oligonucleotide or oligomer having sequence complementary to one or more primer complement portions of an open circle probe, half circle probe, pair of half circle probes, or amplification target circle. This sequence is referred to as the complementary portion of the RCRP. The complementary portion of a RCRP and the cognate primer complement portion can have any desired sequence so long as they are complementary to each other. In general, the sequence of a RCRP can be chosen such that it is not significantly complementary to any other portion of the OCP, HCP, or ATC. That is, the RCRP would be complementary only to primer complement portions. The sequence of a RCRP also can be chosen such that it is not significantly complementary to any portion of a half circle probe in a pair with the half circle probe having the primer complement portion. If random or degenerate rolling circle replication primers are used, the primers collectively will be complementary to many sequences on an ATC, OCP, or HCP. The complementary portion of a rolling circle replication primer can be any length that supports specific and stable hybridization between the primer and the primer complement portion. Generally this is 10 to 35 nucleotides long, but is preferably 16 to 20 nucleotides long. Random or degenerate rolling circle replication primers are preferably 4 to 10 nucleotides long, and most preferably 6, 7 or 8 nucleotides long. Useful rolling circle replication primers are fluorescent change primers. Guide oligonucleotides can serve as rolling circle replication primers. In such cases, the guide oligonucleotide would have a free 3′ end and sequence at the 3′ end of the guide oligonucleotide would be complementary to an amplification target circle.

It is preferred that rolling circle replication primers also contain additional sequence at the 5′ end of the RCRP that is not complementary to any part of the OCP, HCP, or ATC. This sequence is referred to as the non-complementary portion of the RCRP. The non-complementary portion of the RCRP, if present, can serve to facilitate strand displacement during DNA replication. The non-complementary portion of a RCRP can be any length, but is generally 1 to 100 nucleotides long, and preferably 4 to 8 nucleotides long. The non-complementary portion can be involved in interactions that provide specialized effects. For example, the non-complementary portion can comprise a quencher complement portion that can hybridize to a peptide nucleic acid quencher or peptide nucleic acid fluor or that can form an intramolecular structure. Random or degenerate rolling circle replication primers preferably do not include a non-complementary portion. Rolling circle replication primers can also comprise fluorescent moieties or labels and quenching moieties. Rolling circle replication primers can be capable of forming an intramolecular stem structure involving one or both of the RCRP's ends. Such rolling circle replication primers are referred to herein as hairpin rolling circle replication primers. Primers forming intramolecular stem structures, and their use in rolling circle amplification, are described in U.S. patent application Ser. No. 09/803,713.

Rolling circle replication primers can also include modified nucleotides to make it resistant to exonuclease digestion. For example, the primer can have three or four phosphorothioate linkages between nucleotides at the 3′ and/or 5′ end of the primer. Such nuclease resistant primers allow selective degradation of excess unligated OCP or HCPs and gap oligonucleotides that might otherwise interfere with hybridization of detection probes, address probes, and secondary OCPs to the amplified nucleic acid. A rolling circle replication primer can be used as the tertiary DNA strand displacement primer in strand displacement cascade amplification. Random or degenerate rolling circle replication primers can serve as secondary and tertiary DNA strand displacement primers.

A rolling circle replication primer is specific for, or corresponds to, an open circle probe, a half circle probe, or amplification target circle when the complementary portion of the rolling circle replication primer is complementary to the primer complement portion of the open circle probe, the half circle probe, or amplification target circle. A rolling circle replication primer is not specific for, or does not correspond to, an open circle probe, a half circle probe, or amplification target circle when the complementary portion of the rolling circle replication primer is not substantially complementary to the open circle probe, the half circle probe, or amplification target circle. A complementary portion is not substantially complementary to another sequence if it has a melting temperature 10° C. lower than the melting temperature under the same conditions of a sequence fully complementary to the complementary portion of the rolling circle replication primer.

A rolling circle replication primer is specific for, or corresponds to, a set of open circle probes, a set of half circle probes, or a set of amplification target circles when the complementary portion of the rolling circle replication primer is complementary to the primer complement portion of the open circle probes, the half circle probes, or amplification target circles in the set. A rolling circle replication primer is not specific for, or does not correspond to, a set of open circle probes, a set of half circle probes, or a set of amplification target circles when the complementary portion of the rolling circle replication primer is not substantially complementary to the open circle probes, the half circle probes, or amplification target circles in the set.

F. DNA Strand Displacement Primers

Primers used for secondary DNA strand displacement are referred to herein as DNA strand displacement primers. One form of DNA strand displacement primer, referred to herein as a secondary DNA strand displacement primer, is an oligonucleotide or oligomer having sequence matching part of the sequence of an OCP, HCP, or ATC. This sequence in the secondary DNA strand displacement primer is referred to as the matching portion of the secondary DNA strand displacement primer. The sequence in the OCP, HCP, or ATC that matches the matching portion of the secondary DNA strand displacement primer is referred to as the secondary DNA strand displacement primer matching portion. The matching portion of a secondary DNA strand displacement primer is complementary to sequences in TS-DNA. The matching portion of a secondary DNA strand displacement primer may be complementary to any sequence in TS-DNA. However, it is preferred that it not be complementary TS-DNA sequence matching either the rolling circle replication primers or a tertiary DNA strand displacement primer, if one is being used. This prevents hybridization of the primers to each other.

The matching portion of a secondary DNA strand displacement primer may be complementary to all or a portion of a guide sequence or a target sequence. In this case, it is preferred that the 3′ end nucleotides of the secondary DNA strand displacement primer are complementary to the gap sequence in the guide oligonucleotide or target sequence. It is most preferred that nucleotide at the 3′ end of the secondary DNA strand displacement primer falls complementary to the last nucleotide in the gap sequence of the guide oligonucleotide or target sequence, that is, the 5′ nucleotide in the gap sequence of the target sequence. The matching portion of a secondary DNA strand displacement primer can be any length that supports specific and stable hybridization between the primer and its complement. Generally this is 12 to 35 nucleotides long, but is preferably 18 to 25 nucleotides long.

Secondary DNA strand displacement primers can be specific for, or correspond to, all of the open circle probes, half circle probes, or amplification target circles in an amplification reaction or in a set of open circle probes, set half circle probes, or set of amplification target circles in an amplification reaction. A secondary DNA strand displacement primer is specific for, or corresponds to, an open circle probe, half circle probe, or amplification target circle when the matching portion of the secondary DNA strand displacement primer matches the primer complement portion of the open circle probe, half circle probe, or amplification target circle. A secondary DNA strand displacement primer is not specific for, or does not correspond to, an open circle probe, half circle probe, or amplification target circle when the matching portion of the secondary DNA strand displacement primer does not substantially match sequence in the open circle probe, half circle probe, or amplification target circle. A matching portion does not substantially match another sequence if it has a melting temperature with the complement of the other sequence that is 10° C. lower than the melting temperature under the same conditions of a sequence fully complementary to the matching portion of the secondary DNA strand displacement primer.

A secondary DNA strand displacement primer is specific for, or corresponds to, a set of open circle probes, a set of half circle probes, or a set of amplification target circles when the matching portion of the secondary DNA strand displacement primer matches the primer complement portion of the open circle probes, half circle probes, or amplification target circles in the set. A secondary DNA strand displacement primer is not specific for, or does not correspond to, a set of open circle probes, a set of half circle probes, or a set of amplification target circles when the matching portion of the secondary DNA strand displacement primer does not substantially match the open circle probes, half circle probes, or amplification target circles in the set. Secondary DNA strand displacement primers can be fluorescent change primers although this is not preferred.

It is preferred that secondary DNA strand displacement primers also contain additional sequence at the 5′ end of the primer that does not match any part of the OCP, HCP, or ATC. This sequence is referred to as the non-matching portion of the secondary DNA strand displacement primer. The non-matching portion of the secondary DNA strand displacement primer, if present, can serve to facilitate strand displacement during DNA replication. The non-matching portion of a secondary DNA strand displacement primer may be any length, but is generally 1 to 100 nucleotides long, and preferably 4 to 8 nucleotides long. The non-matching portion can be involved in interactions that provide specialized effects. For example, the non-matching portion can comprise a quencher complement portion that can hybridize to a peptide nucleic acid quencher or peptide nucleic acid fluor or that can form an intramolecular structure. Secondary DNA strand displacement primers can also comprise fluorescent moieties or labels and quenching moieties.

Useful secondary DNA strand displacement primers for use in the disclosed method can form an intramolecular stem structure involving one or both of the secondary DNA strand displacement primer's ends. Such secondary DNA strand displacement primers are referred to herein as hairpin secondary DNA strand displacement primers. Primers forming intramolecular stem structures, and their use in rolling circle amplification, are described in U.S. patent application Ser. No. 09/803,713.

Another form of DNA strand displacement primer, referred to herein as a tertiary DNA strand displacement primer, is an oligonucleotide having sequence complementary to part of the sequence of an OCP, HCP, or ATC. This sequence is referred to as the complementary portion of the tertiary DNA strand displacement primer. This complementary portion of the tertiary DNA strand displacement primer matches sequences in TS-DNA. The complementary portion of a tertiary DNA strand displacement primer may be complementary to any sequence in the OCP, HCP, or ATC. However, it is preferred that it not be complementary to the OCP, HCP, or ATC sequence matching the secondary DNA strand displacement primer. This prevents hybridization of the primers to each other. Preferably, the complementary portion of the tertiary DNA strand displacement primer has sequence complementary to a portion of the spacer portion of an OCP, HCP, or ATC. The complementary portion of a tertiary DNA strand displacement primer can be any length that supports specific and stable hybridization between the primer and its complement. Generally this is 12 to 35 nucleotides long, but is preferably 18 to 25 nucleotides long. Tertiary DNA strand displacement primers can be fluorescent change primers although this is not preferred.

Useful tertiary DNA strand displacement primers for use in the disclosed method can form an intramolecular stem structure involving one or both of the tertiary DNA strand displacement primer's ends. Such tertiary DNA strand displacement primers are referred to herein as hairpin tertiary DNA strand displacement primers.

It is preferred that tertiary DNA strand displacement primers also contain additional sequence at their 5′ end that is not complementary to any part of the OCP, HCP, or ATC. This sequence is referred to as the non-complementary portion of the tertiary DNA strand displacement primer. The non-complementary portion of the tertiary DNA strand displacement primer, if present, serves to facilitate strand displacement during DNA replication. The non-complementary portion of a tertiary DNA strand displacement primer may be any length, but is generally 1 to 100 nucleotides long, and preferably 4 to 8 nucleotides long. A rolling circle replication primer is a preferred form of tertiary DNA strand displacement primer. Tertiary DNA strand displacement primers can also comprise fluorescent moieties or labels and quenching moieties.

DNA strand displacement primers may also include modified nucleotides to make them resistant to exonuclease digestion. For example, the primer can have three or four phosphorothioate linkages between nucleotides at the 3′ and/or 5′ end of the primer. Such nuclease resistant primers allow selective degradation of excess unligated OCP or HCP and gap oligonucleotides that might otherwise interfere with hybridization of detection probes, address probes, and secondary OCPs to the amplified nucleic acid. DNA strand displacement primers can be used for secondary DNA strand displacement and strand displacement cascade amplification, both described below and in U.S. Pat. No. 6,143,495.

G. Fluorescent Change Probes and Primers

Fluorescent change probes and fluorescent change primers refer to all probes and primers that involve a change in fluorescence intensity or wavelength based on a change in the form or conformation of the probe or primer and nucleic acid to be detected, assayed or replicated. Examples of fluorescent change probes and primers include molecular beacons, Amplifluors, FRET probes, cleavable FRET probes, TaqMan probes, scorpion primers, fluorescent triplex oligos including but not limited to triplex molecular beacons or triplex FRET probes, fluorescent water-soluble conjugated polymers, PNA probes and QPNA probes.

Fluorescent change probes and primers can be classified according to their structure and/or function. Fluorescent change probes include hairpin quenched probes, cleavage quenched probes, cleavage activated probes, and fluorescent activated probes. Fluorescent change primers include stem quenched primers and hairpin quenched primers. The use of several types of fluorescent change probes and primers are reviewed in Schweitzer and Kingsmore, Curr. Opin. Biotech. 12:21-27 (2001). Hall et al., Proc. Natl. Acad. Sci. USA 97:8272-8277 (2000), describe the use of fluorescent change probes with Invader assays.

Hairpin quenched probes are probes that when not bound to a target sequence form a hairpin structure (and, typically, a loop) that brings a fluorescent label and a quenching moiety into proximity such that fluorescence from the label is quenched. When the probe binds to a target sequence, the stem is disrupted, the quenching moiety is no longer in proximity to the fluorescent label and fluorescence increases. Examples of hairpin quenched probes are molecular beacons, fluorescent triplex oligos, triplex molecular beacons, triplex FRET probes, and QPNA probes.

Cleavage activated probes are probes where fluorescence is increased by cleavage of the probe. Cleavage activated probes can include a fluorescent label and a quenching moiety in proximity such that fluorescence from the label is quenched. When the probe is clipped or digested (typically by the 5′-3′ exonuclease activity of a polymerase during amplification), the quenching moiety is no longer in proximity to the fluorescent label and fluorescence increases. TaqMan probes (Holland et al., Proc. Natl. Acad. Sci. USA 88:7276-7280 (1991)) are an example of cleavage activated probes.

Cleavage quenched probes are probes where fluorescence is decreased or altered by cleavage of the probe. Cleavage quenched probes can include an acceptor fluorescent label and a donor moiety such that, when the acceptor and donor are in proximity, fluorescence resonance energy transfer from the donor to the acceptor causes the acceptor to fluoresce. The probes are thus fluorescent, for example, when hybridized to a target sequence. When the probe is clipped or digested (typically by the 5′-3′ exonuclease activity of a polymerase during amplification), the donor moiety is no longer in proximity to the acceptor fluorescent label and fluorescence from the acceptor decreases. If the donor moiety is itself a fluorescent label, it can release energy as fluorescence (typically at a different wavelength than the fluorescence of the acceptor) when not in proximity to an acceptor. The overall effect would then be a reduction of acceptor fluorescence and an increase in donor fluorescence. Donor fluorescence in the case of cleavage quenched probes is equivalent to fluorescence generated by cleavage activated probes with the acceptor being the quenching moiety and the donor being the fluorescent label. Cleavable FRET (fluorescence resonance energy transfer) probes are an example of cleavage quenched probes.

Fluorescent activated probes are probes or pairs of probes where fluorescence is increased or altered by hybridization of the probe to a target sequence. Fluorescent activated probes can include an acceptor fluorescent label and a donor moiety such that, when the acceptor and donor are in proximity (when the probes are hybridized to a target sequence), fluorescence resonance energy transfer from the donor to the acceptor causes the acceptor to fluoresce. Fluorescent activated probes are typically pairs of probes designed to hybridize to adjacent sequences such that the acceptor and donor are brought into proximity. Fluorescent activated probes can also be single probes containing both a donor and acceptor where, when the probe is not hybridized to a target sequence, the donor and acceptor are not in proximity but where the donor and acceptor are brought into proximity when the probe hybridized to a target sequence. This can be accomplished, for example, by placing the donor and acceptor on opposite ends of the probe and placing target complement sequences at each end of the probe where the target complement sequences are complementary to adjacent sequences in a target sequence. If the donor moiety of a fluorescent activated probe is itself a fluorescent label, it can release energy as fluorescence (typically at a different wavelength than the fluorescence of the acceptor) when not in proximity to an acceptor (that is, when the probes are not hybridized to the target sequence). When the probes hybridize to a target sequence, the overall effect would then be a reduction of donor fluorescence and an increase in acceptor fluorescence. FRET probes are an example of fluorescent activated probes.

Stem quenched primers are primers that when not hybridized to a complementary sequence form a stem structure (either an intramolecular stem structure or an intermolecular stem structure) that brings a fluorescent label and a quenching moiety into proximity such that fluorescence from the label is quenched. When the primer binds to a complementary sequence, the stem is disrupted, the quenching moiety is no longer in proximity to the fluorescent label and fluorescence increases. In the disclosed method, stem quenched primers are used as primers for nucleic acid synthesis and thus become incorporated into the synthesized or amplified nucleic acid. Examples of stem quenched primers are peptide nucleic acid quenched primers and hairpin quenched primers.

Peptide nucleic acid quenched primers are primers associated with a peptide nucleic acid quencher or a peptide nucleic acid fluor to form a stem structure. The primer contains a fluorescent label or a quenching moiety and is associated with either a peptide nucleic acid quencher or a peptide nucleic acid fluor, respectively. This puts the fluorescent label in proximity to the quenching moiety. When the primer is replicated, the peptide nucleic acid is displaced, thus allowing the fluorescent label to produce a fluorescent signal.

Hairpin quenched primers are primers that when not hybridized to a complementary sequence form a hairpin structure (and, typically, a loop) that brings a fluorescent label and a quenching moiety into proximity such that fluorescence from the label is quenched. When the primer binds to a complementary sequence, the stem is disrupted, the quenching moiety is no longer in proximity to the fluorescent label and fluorescence increases. Hairpin quenched primers are typically used as primers for nucleic acid synthesis and thus become incorporated into the synthesized or amplified nucleic acid. Examples of hairpin quenched primers are Amplifluor primers (Nazerenko et al., Nucleic Acids Res. 25:2516-2521 (1997)) and scorpion primers (Thelwell et al., Nucleic Acids Res. 28(19):3752-3761 (2000)).

Cleavage activated primers are similar to cleavage activated probes except that they are primers that are incorporated into replicated strands and are then subsequently cleaved. Little et al., Clin. Chem. 45:777-784 (1999), describe the use of cleavage activated primers.

H. Analytes

Some forms of the disclosed method involve labeling or detection of analytes. In general, any compound, moiety, or component of a compound or complex can be an analyte. Preferred analytes are peptides, proteins, and other macromolecules such as lipids, complex carbohydrates, proteolipids, membrane fragments, and nucleic acids. Analytes can also be smaller molecules such as cofactors, metabolites, enzyme substrates, metal ions, and metal chelates. Analytes preferably range in size from 100 daltons to 1,000,000 daltons. It is to be understood that the term analyte refers to both separate molecules and to portions of molecules, such as an epitope of a protein, that interacts specifically with an analyte interaction portion.

Analytes may contain modifications, both naturally occurring or induced in vitro or in vivo. Induced modifications include adduct formation such as hapten attachment, multimerization, complex formation by interaction with other chemical moieties, digestion or cleavage (by, for example, protease), and metal ion attachment or removal. The disclosed method can be used to detect differences in the modification state of an analyte, such as the phosphorylation or glycosylation state of proteins.

Analytes can be associated directly or indirectly with substrates, preferably in arrays. Most preferred are microarrays. Analytes can be captured and/or immobilized using analyte capture agents. Immobilized analytes can be used to capture other components used in the disclosed method such as analyte capture agents and reporter binding primers. Samples that contain or that may contain analytes can be referred to as analyte samples.

I. Detection Labels

To aid in detection and quantitation of nucleic acids amplified using the disclosed method, detection labels can be directly incorporated into amplified nucleic acids or can be coupled to detection molecules. As used herein, a detection label is any molecule that can be associated with amplified nucleic acid, directly or indirectly, and which results in a measurable, detectable signal, either directly or indirectly. Many such labels for incorporation into nucleic acids or coupling to nucleic acid probes are known to those of skill in the art. Examples of detection labels suitable for use in the disclosed method are radioactive isotopes, fluorescent molecules, phosphorescent molecules, enzymes, antibodies, and ligands. Fluorescent labels, especially in the context of fluorescent change probes and primers, are useful for real-time detection of amplification.

Examples of suitable fluorescent labels include fluorescein isothiocyanate (FITC), 5,6-carboxymethyl fluorescein, Texas red, nitrobenz-2-oxa-1,3-diazol-4-yl (NBD), coumarin, dansyl chloride, rhodamine, amino-methyl coumarin (AMCA), Eosin, Erythrosin, BODIPY®, Cascade Blue®, Oregon Green®, pyrene, lissamine, xanthenes, acridines, oxazines, phycoerythrin, macrocyclic chelates of lanthanide ions such as quantum dye™, fluorescent energy transfer dyes, such as thiazole orange-ethidium heterodimer, and the cyanine dyes Cy3, Cy3.5, Cy5, Cy5.5 and Cy7. Examples of other specific fluorescent labels include 3-Hydroxypyrene 5,8,10-Tri Sulfonic acid, 5-Hydroxy Tryptamine (5-HT), Acid Fuchsin, Alizarin Complexon, Alizarin Red, Allophycocyanin, Aminocoumarin, Anthroyl Stearate, Astrazon Brilliant Red 4G, Astrazon Orange R, Astrazon Red 6B, Astrazon Yellow 7 GLL, Atabrine, Auramine, Aurophosphine, Aurophosphine G, BAO 9 (Bisaminophenyloxadiazole), BCECF, Berberine Sulphate, Bisbenzamide, Blancophor FFG Solution, Blancophor SV, Bodipy F1, Brilliant Sulphoflavin FF, Calcien Blue, Calcium Green, Calcofluor RW Solution, Calcofluor White, Calcophor White ABT Solution, Calcophor White Standard Solution, Carbostyryl, Cascade Yellow, Catecholamine, Chinacrine, Coriphosphine O, Coumarin-Phalloidin, CY3.1 8, CY5.1 8, CY7, Dans (1-Dimethyl Amino Naphaline 5 Sulphonic Acid), Dansa (Diamino Naphtyl Sulphonic Acid), Dansyl NH-CH3, Diamino Phenyl Oxydiazole (DAO), Dimethylamino-5-Sulphonic acid, Dipyrrometheneboron Difluoride, Diphenyl Brilliant Flavine 7GFF, Dopamine, Erythrosin ITC, Euchrysin, FIF (Formaldehyde Induced Fluorescence), Flazo Orange, Fluo 3, Fluorescamine, Fura-2, Genacryl Brilliant Red B, Genacryl Brilliant Yellow 10GF, Genacryl Pink 3G, Genacryl Yellow 5GF, Gloxalic Acid, Granular Blue, Haematoporphyrin, Indo-1, Intrawhite Cf Liquid, Leucophor PAF, Leucophor SF, Leucophor WS, Lissamine Rhodamine B200 (RD200), Lucifer Yellow CH, Lucifer Yellow VS, Magdala Red, Marina Blue, Maxilon Brilliant Flavin 10 GFF, Maxilon Brilliant Flavin 8 GFF, MPS (Methyl Green Pyronine Stilbene), Mithramycin, NBD Amine, Nitrobenzoxadidole, Noradrenaline, Nuclear Fast Red, Nuclear Yellow, Nylosan Brilliant Flavin E8G, Oxadiazole, Pacific Blue, Pararosaniline (Feulgen), Phorwite AR Solution, Phorwite BKL, Phorwite Rev, Phorwite RPA, Phosphine 3R, Phthalocyanine, Phycoerythrin R, Phycoerythrin B, Polyazaindacene Pontochrome Blue Black, Porphyrin, Primuline, Procion Yellow, Pyronine, Pyronine B, Pyrozal Brilliant Flavin 7GF, Quinacrine Mustard, Rhodamine 123, Rhodamine 5 GLD, Rhodamine 6G, Rhodamine B, Rhodamine B 200, Rhodamine B Extra, Rhodamine BB, Rhodamine BG, Rhodamine WT, Serotonin, Sevron Brilliant Red 2B, Sevron Brilliant Red 4G, Sevron Brilliant Red B, Sevron Orange, Sevron Yellow L, SITS (Primuline), SITS (Stilbene Isothiosulphonic acid), Stilbene, Snarf 1, sulpho Rhodamine B Can C, Sulpho Rhodamine G Extra, Tetracycline, Thiazine Red R, Thioflavin S, Thioflavin TCN, Thioflavin 5, Thiolyte, Thiozol Orange, Tinopol CBS, True Blue, Ultralite, Uranine B, Uvitex SFC, Xylene Orange, and XRITC.

Preferred fluorescent labels are fluorescein (5-carboxyfluorescein-N-hydroxysuccinimide ester), rhodamine (5,6-tetramethyl rhodamine), and the cyanine dyes Cy3, Cy3.5, Cy5, Cy5.5 and Cy7. The absorption and emission maxima, respectively, for these fluors are: FITC (490 nm; 520 nm), Cy3 (554 nm; 568 nm), Cy3.5 (581 nm; 588 nm), Cy5 (652 nm: 672 mn), Cy5.5 (682 nm; 703 nm) and Cy7 (755 nm; 778 nm), thus allowing their simultaneous detection. Other examples of fluorescein dyes include 6-carboxyfluorescein (6-FAM), 2′,4′,1,4,-tetrachlorofluorescein (TET), 2′,4′,5′,7′,1,4-hexachlorofluorescein (HEX), 2′,7′-dimethoxy-4′,5′-dichloro-6-carboxyrhodamine (JOE), 2′-chloro-5′-fluoro-7′,8′-fused phenyl-1,4-dichloro-6-carboxyfluorescein (NED), and 2′-chloro-7′-phenyl-1,4-dichloro-6-carboxyfluorescein (VIC). Fluorescent labels can be obtained from a variety of commercial sources, including Amersham Pharmacia Biotech, Piscataway, N.J.; Molecular Probes, Eugene, Oreg.; and Research Organics, Cleveland, Ohio.

Additional labels of interest include those that provide for signal only when the probe with which they are associated is specifically bound to a target molecule, where such labels include: “molecular beacons” as described in Tyagi & Kramer, Nature Biotechnology (1996) 14:303 and EP 0 070 685 B1. Other labels of interest include those described in U.S. Pat. No. 5,563,037; WO 97/17471 and WO 97/17076.

Labeled nucleotides are a preferred form of detection label since they can be directly incorporated into the amplification products during synthesis. Examples of detection labels that can be incorporated into amplified nucleic acids include nucleotide analogs such as BrdUrd (5-bromodeoxyuridine, Hoy and Schimke, Mutation Research 290:217-230 (1993)), aminoallyldeoxyuridine (Henegariu et al., Nature Biotechnology 18:345-348 (2000)), 5-methylcytosine (Sano et al., Biochim. Biophys. Acta 951:157-165 (1988)), bromouridine (Wansick et al., J. Cell Biology 122:283-293 (1993)) and nucleotides modified with biotin (Langer et al., Proc. Natl. Acad. Sci. USA 78:6633 (1981)) or with suitable haptens such as digoxygenin (Kerkhof, Anal. Biochem. 205:359-364 (1992)). Suitable fluorescence-labeled nucleotides are Fluorescein-isothiocyanate-dUTP, Cyanine-3-dUTP and Cyanine-5-dUTP (Yu et al., Nucleic Acids Res., 22:3226-3232 (1994)). A preferred nucleotide analog detection label for DNA is BrdUrd (bromodeoxyuridine, BrdUrd, BrdU, BUdR, Sigma-Aldrich Co). Other preferred nucleotide analogs for incorporation of detection label into DNA are AA-dUTP (aminoallyl-deoxyuridine triphosphate, Sigma-Aldrich Co.), and 5-methyl-dCTP (Roche Molecular Biochemicals). A preferred nucleotide analog for incorporation of detection label into RNA is biotin-16-UTP (biotin-16-uridine-5′-triphosphate, Roche Molecular Biochemicals). Fluorescein, Cy3, and Cy5 can be linked to dUTP for direct labeling. Cy3.5 and Cy7 are available as avidin or anti-digoxygenin conjugates for secondary detection of biotin- or digoxygenin-labeled probes.

Detection labels that are incorporated into amplified nucleic acid, such as biotin, can be subsequently detected using sensitive methods well-known in the art. For example, biotin can be detected using streptavidin-alkaline phosphatase conjugate (Tropix, Inc.), which is bound to the biotin and subsequently detected by chemiluminescence of suitable substrates (for example, chemiluminescent substrate CSPD: disodium, 3-(4-methoxyspiro-[1,2,-dioxetane-3-2′-(5′-chloro)tricyclo [3.3.1.1^{3,7}]decane]-4-yl) phenyl phosphate; Tropix, Inc.). Labels can also be enzymes, such as alkaline phosphatase, soybean peroxidase, horseradish peroxidase and polymerases, that can be detected, for example, with chemical signal amplification or by using a substrate to the enzyme which produces light (for example, a chemiluminescent 1,2-dioxetane substrate) or fluorescent signal.

Molecules that combine two or more of these detection labels are also considered detection labels. Any of the known detection labels can be used with the disclosed probes, tags, and method to label and detect nucleic acid amplified using the disclosed method. Methods for detecting and measuring signals generated by detection labels are also known to those of skill in the art. For example, radioactive isotopes can be detected by scintillation counting or direct visualization; fluorescent molecules can be detected with fluorescent spectrophotometers; phosphorescent molecules can be detected with a spectrophotometer or directly visualized with a camera; enzymes can be detected by detection or visualization of the product of a reaction catalyzed by the enzyme; antibodies can be detected by detecting a secondary detection label coupled to the antibody. As used herein, detection molecules are molecules which interact with amplified nucleic acid and to which one or more detection labels are coupled.

J. Detection Probes

Detection probes are labeled oligonucleotides or oligomers having sequence complementary to detection tags on TS-DNA or transcripts of TS-DNA. The complementary portion of a detection probe can be any length that supports specific and stable hybridization between the detection probe and the detection tag. For this purpose, a length of 10 to 35 nucleotides is preferred, with a complementary portion of a detection probe 16 to 20 nucleotides long being most preferred. Detection probes can contain any of the detection labels described above. Preferred labels are biotin and fluorescent molecules. Useful detection probes are fluorescent change probes. A particularly preferred detection probe is a molecular beacon (which is a form of fluorescent change probe). Molecular beacons are detection probes labeled with fluorescent moieties where the fluorescent moieties fluoresce only when the detection probe is hybridized (Tyagi and Kramer, Nature Biotechnology 14:303-308 (1996)). The use of such probes eliminates the need for removal of unhybridized probes prior to label detection because the unhybridized detection probes will not produce a signal. This is especially useful in multiplex assays.

One form of detection probe, referred to herein as a collapsing detection probe, contains two separate complementary portions. This allows each detection probe to hybridize to two detection tags in TS-DNA. In this way, the detection probe forms a bridge between different parts of the TS-DNA. The combined action of numerous collapsing detection probes hybridizing to TS-DNA will be to form a collapsed network of cross-linked TS-DNA. Collapsed TS-DNA occupies a much smaller volume than free, extended TS-DNA, and includes whatever detection label present on the detection probe. This result is a compact and discrete detectable signal for each TS-DNA. Collapsing TS-DNA is useful both for in situ hybridization applications and for multiplex detection because it allows detectable signals to be spatially separate even when closely packed. Collapsing TS-DNA is described in U.S. Pat. No. 6,143,495.

K. Gap Oligonucleotides

Gap oligonucleotides are oligonucleotides that are complementary to all or a part of that portion of a target sequence or guide oligonucleotide which covers a gap space between the ends of a hybridized open circle probe or between the ends of a hybridized pair of half circle probes. Gap oligonucleotides have a phosphate group at their 5′ ends and a hydroxyl group at their 3′ ends. This facilitates ligation of gap oligonucleotides to open circle probes, half circle probes, or to other gap oligonucleotides. The gap space between the ends of a hybridized open circle probe or between the ends of a hybridized pair of half circle probes can be filled with a single gap oligonucleotide, or it can be filled with multiple gap oligonucleotides. For example, two 3 nucleotide gap oligonucleotides can be used to fill a six nucleotide gap space, or a three nucleotide gap oligonucleotide and a four nucleotide gap oligonucleotide can be used to fill a seven nucleotide gap space. Gap oligonucleotides are particularly useful for distinguishing between closely related target sequences and guide sequences. For example, multiple gap oligonucleotides can be used to amplify different allelic variants of a target sequence. By placing the region of the target sequence or guide sequence in which the variation occurs in the gap space formed by an open circle probe or a pair of half circle probes, a single open circle probe or pair of half circle probes can be used to amplify each of the individual variants by using an appropriate set of gap oligonucleotides.

L. Address Probes

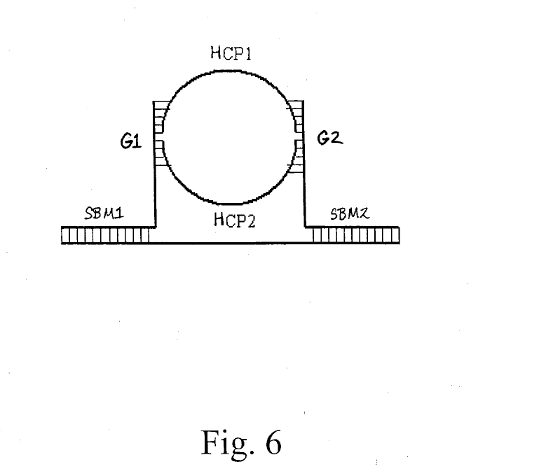

An address probe is an oligonucleotide or oligomer having a sequence complementary to address tags on TS-DNA or transcripts of TS-DNA. The complementary portion of an address probe can be any length that supports specific and stable hybridization between the address probe and the address tag. For this purpose, a length of 10 to 35 nucleotides is preferred, with a complementary portion of an address probe 12 to 18 nucleotides long being most preferred. Preferably, the complementary portion of an address probe is complementary to all or a portion of the target probe portions of an OCP, a guide complement portion of an HCP, or the guide complement portions of a pair of HCPs. Most preferably, the complementary portion of an address probe is complementary to a portion of either or both of the left and right target probe portions of an OCP, a portion of either or both of the 5′ guide complement portion of one HCP in a pair of HCPs and the 3′ guide complement portion of the other HCP in the pair of HCPs, as well as all or a part of any gap oligonucleotides or gap sequence created in a gap-filling operation (see FIG. 6 of U.S. Pat. No. 6,143,495). Address probes can contain a single complementary portion or multiple complementary portions. Preferably, address probes are coupled, either directly or via a spacer molecule, to a solid-state support. Such a combination of address probe and solid-state support are a preferred form of solid-state detector. Address probes can be fluorescent change probes although this is not preferred.

M. Solid Supports

Solid supports are solid-state substrates or supports with which target molecules or amplification products of the disclosed method (or other components used in, or produced by, the disclosed method) can be associated. Target molecules and amplification products can be associated with solid supports directly or indirectly. For example, analytes can be bound to the surface of a solid support or associated with analyte capture agents immobilized on solid supports. As another example, amplification products can be bound to the surface of a solid support or associated with address probes, or detection probes immobilized on solid supports. An array detector is a solid support to which multiple different address probes or detection probes have been associated in an array, grid, or other organized pattern. A target array is a solid support to which multiple different target molecules, target sequences, analytes, and/or analyte capture agents have been associated in an array, grid, or other organized pattern. Binding guide conjugates, binding guide analytes, test analytes and other components used in the disclosed methods can also be immobilized on a solid support.

Solid-state substrates for use in solid supports can include any solid material with which components can be associated, directly or indirectly. This includes materials such as acrylamide, agarose, cellulose, nitrocellulose, glass, gold, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, functionalized silane, polypropylfumerate, collagen, glycosaminoglycans, and polyamino acids. Solid-state substrates can have any useful form including thin film, membrane, bottles, dishes, fibers, woven fibers, shaped polymers, particles, beads, microparticles, or a combination. Solid-state substrates and solid supports can be porous or non-porous. A chip is a rectangular or square small piece of material. Preferred forms for solid-state substrates are thin films, beads, or chips. A useful form for a solid-state substrate is a microtiter dish. In some embodiments, a multiwell glass slide can be employed.

Different target molecules, target sequences, analytes, analyte capture agents, address probes and/or detection probes can be used together as a set. The set can be used as a mixture of all or subsets of the analytes, analyte capture agents, address probes and/or detection probes used separately in separate reactions, or immobilized on a solid support. Target molecules, target sequences, analytes, analyte capture agents, address probes and/or detection probes used separately or as mixtures can be physically separable through, for example, association with or immobilization on a solid support. An array can include a plurality of target molecules, target sequences, analytes, analyte capture agents, address probes and/or detection probes immobilized at identified or predefined locations on the solid support. Each predefined location on the solid support generally has one type of component (that is, all the components at that location are the same). Alternatively, multiple types of components can be immobilized in the same predefined location on a solid support. Each location will have multiple copies of the given components. The spatial separation of different components on the solid support allows separate detection and identification of amplification products.

Although useful, it is not required that the solid support be a single unit or structure. The set of target molecules, target sequences, analytes, analyte capture agents, address probes and/or detection probes may be distributed over any number of solid supports. For example, at one extreme, each component may be immobilized in a separate reaction tube or container, or on separate beads or microparticles.

Methods for immobilization of oligonucleotides to solid-state substrates are well established. Oligonucleotides, including address probes and detection probes, can be coupled to substrates using established coupling methods. For example, suitable attachment methods are described by Pease et al., Proc. Natl. Acad. Sci. USA 91(11):5022-5026 (1994), and Khrapko et al., Mol Biol (Mosk) (USSR) 25:718-730 (1991). A method for immobilization of 3′-amine oligonucleotides on casein-coated slides is described by Stimpson et al., Proc. Natl. Acad. Sci. USA 92:6379-6383 (1995). A useful method of attaching oligonucleotides to solid-state substrates is described by Guo et al., Nucleic Acids Res. 22:5456-5465 (1994).

Each of the components (for example, analyte capture agents, address probes and/or detection probes) immobilized on the solid support can be located in a different predefined region of the solid support. The different locations can be different reaction chambers. Each of the different predefined regions can be physically separated from each other of the different regions. The distance between the different predefined regions of the solid support can be either fixed or variable. For example, in an array, each of the components can be arranged at fixed distances from each other, while components associated with beads will not be in a fixed spatial relationship. In particular, the use of multiple solid support units (for example, multiple beads) will result in variable distances.

Components can be associated or immobilized on a solid support at any density. Components can be immobilized to the solid support at a density exceeding 400 different components per cubic centimeter. Arrays of components can have any number of components. For example, an array can have at least 1,000 different components immobilized on the solid support, at least 10,000 different components immobilized on the solid support, at least 100,000 different components immobilized on the solid support, or at least 1,000,000 different components immobilized on the solid support.

N. Solid-State Detectors

Solid-state detectors are solid supports to which address probes or detection molecules have been associated. A preferred form of solid-state detector is an array detector. An array detector is a solid-state detector to which multiple different address probes or detection molecules have been associated in an array, grid, or other organized pattern. Another form of solid-state detector is a target array. A target array is a solid support to which multiple different target molecules, target sequences, analytes, and/or analyte capture agents have been associated in an array, grid, or other organized pattern.

Solid-state substrates for use in solid-state detectors can include any solid material to which oligonucleotides can be coupled. This includes materials such as acrylamide, agarose, cellulose, nitrocellulose, glass, gold, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, functionalized silane, polypropylfumerate, collagen, glycosaminoglycans, and polyamino acids. Solid-state substrates can have any useful form including thin film, membrane, bottles, dishes, fibers, woven fibers, shaped polymers, particles, beads, microparticles, or a combination. Solid-state substrates and solid supports can be porous or non-porous. A chip is a rectangular or square small piece of material. Preferred forms for solid-state substrates are thin films, beads, or chips. A useful form for a solid-state substrate is a microtiter dish. In some embodiments, a multiwell glass slide can be employed.

Address probes immobilized on a solid-state substrate allow capture of the products of the disclosed amplification method on a solid-state detector. Such capture provides a convenient means of washing away reaction components that might interfere with subsequent detection steps. By attaching different address probes to different regions of a solid-state detector, different amplification products can be captured at different, and therefore diagnostic, locations on the solid-state detector. For example, in a multiplex assay, address probes specific for numerous different amplified nucleic acids (each representing a different target sequence amplified via a different set of primers) can be immobilized in an array, each in a different location. Capture and detection will occur only at those array locations corresponding to amplified nucleic acids for which the corresponding target sequences were present in a sample.

Methods for immobilization of oligonucleotides to solid-state substrates are well established. Oligonucleotides, including address probes and detection probes, can be coupled to substrates using established coupling methods. For example, suitable attachment methods are described by Pease et al., Proc. Natl. Acad. Sci. USA 91(11):5022-5026 (1994), and Khrapko et al., Mol Biol (Mosk) (USSR) 25:718-730 (1991). A method for immobilization of 3′-amine oligonucleotides on casein-coated slides is described by Stimpson et al., Proc. Natl. Acad. Sci. USA 92:6379-6383 (1995). A preferred method of attaching oligonucleotides to solid-state substrates is described by Guo et al., Nucleic Acids Res. 22:5456-5465 (1994). Examples of nucleic acid chips and arrays, including methods of making and using such chips and arrays, are described in U.S. Pat. Nos. 6,287,768, 6,288,220, 6,287,776, 6,297,006, and 6,291,193.

O. Solid-State Samples

Solid-state samples are solid supports to which target molecules, target sequences, analytes, or analyte capture agents have been associated. These components preferably can be delivered in a target sample, analyte sample, or assay sample. One form of solid-state sample is an array sample. An array sample is a solid-state sample to which multiple different target samples or assay samples have been coupled or adhered in an array, grid, or other organized pattern. Another form of solid-state detector is a target array. A target array is a solid support to which multiple different target molecules, target sequences, analytes, and/or analyte capture agents have been associated in an array, grid, or other organized pattern. Components of a solid-state sample can be associated with the solid support in any suitable manner, including coupling, adhering, immobilizing, binding, and other covalent and non-covalent interactions. Target molecules, target sequences, analytes, and analyte capture agents can be associated with the solid support directly or indirectly.

Solid-state substrates for use in solid-state samples can include any solid material to which target molecules or target sequences can be coupled or adhered. This includes materials such as acrylamide, agarose, cellulose, nitrocellulose, glass, gold, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, functionalized silane, polypropylfumerate, collagen, glycosaminoglycans, and polyamino acids. Solid-state substrates can have any useful form including thin film, membrane, bottles, dishes, fibers, woven fibers, shaped polymers, particles, beads, microparticles, or a combination. Solid-state substrates and solid supports can be porous or non-porous. A chip is a rectangular or square small piece of material. Preferred forms for solid-state substrates are thin films, beads, or chips. A useful form for a solid-state substrate is a microtiter dish. In some embodiments, a multiwell glass slide can be employed.

Immobilization of target molecules, target sequences, analytes, and analyte capture agents on a solid-state substrate allows formation of target-specific TS-DNA localized on the solid-state substrate. Such localization provides a convenient means of washing away reaction components that might interfere with subsequent detection steps, and a convenient way of assaying multiple different samples and target molecules simultaneously. Diagnostic TS-DNA can be independently formed at each site where a different sample is adhered. For immobilization of target sequences or other oligonucleotide molecules to form a solid-state sample, the methods described above for can be used. Nucleic acids produced in the disclosed method can be coupled or adhered to a solid-state substrate in any suitable way. For example, nucleic acids generated by multiple strand displacement can be attached by adding modified nucleotides to the 3′ ends of nucleic acids produced by strand displacement replication using terminal deoxynucleotidyl transferase, and reacting the modified nucleotides with a solid-state substrate or support thereby attaching the nucleic acids to the solid-state substrate or support.

One form of solid-state sample useful in the disclosed method has analyte capture agents attached to a solid-state substrate. Such analyte capture agents can be specific for a target molecule. Captured target molecules can then be detected by binding of binding guide conjugates, circularization of half circle probes, and amplification. Such a use of analyte capture agents in a solid-state detector allows assays to be developed for the detection of any molecule for which analyte capture agents can be generated. Preferred analyte capture agents are proteins. Methods for immobilizing antibodies and other proteins to solid-state substrates are well established. Immobilization can be accomplished by attachment, for example, to aminated surfaces, carboxylated surfaces or hydroxylated surfaces using standard immobilization chemistries. Examples of attachment agents are cyanogen bromide, succinimide, aldehydes, tosyl chloride, avidin-biotin, photocrosslinkable agents, epoxides and maleimides. A preferred attachment agent is glutaraldehyde. These and other attachment agents, as well as methods for their use in attachment, are described in Protein immobilization: fundamentals and applications, Richard F. Taylor, ed. (M. Dekker, New York, 1991), Johnstone and Thorpe, Immunochemistry In Practice (Blackwell Scientific Publications, Oxford, England, 1987) pages 209-216 and 241-242, and Immobilized Affinity Ligands, Craig T. Hermanson et al., eds. (Academic Press, New York, 1992). Antibodies and other proteins can be attached to a substrate by chemically cross-linking a free amino group on the antibody or protein to reactive side groups present within the solid-state substrate. For example, antibodies may be chemically cross-linked to a substrate that contains free amino or carboxyl groups using glutaraldehyde or carbodiimides as cross-linker agents. In this method, aqueous solutions containing free antibodies are incubated with the solid-state substrate in the presence of glutaraldehyde or carbodiimide. For crosslinking with glutaraldehyde the reactants can be incubated with 2% glutaraldehyde by volume in a buffered solution such as 0.1 M sodium cacodylate at pH 7.4. Other standard immobilization chemistries are known by those of skill in the art.

One form of solid-state substrate is a glass slide to which up to 256 separate target samples have been adhered as an array of small dots. Each dot is preferably from 0.1 to 2.5 mm in diameter, and most preferably around 2.5 mm in diameter. Such microarrays can be fabricated, for example, using the method described by Schena et al., Science 270:487-470 (1995). Briefly, microarrays can be fabricated on poly-L-lysine-coated microscope slides (Sigma) with an arraying machine fitted with one printing tip. The tip is loaded with 1 μl of a DNA sample (0.5 mg/ml) from, for example, 96-well microtiter plates and deposited ˜0.005 μl per slide on multiple slides at the desired spacing. The printed slides can then be rehydrated for 2 hours in a humid chamber, snap-dried at 100° C. for 1 minute, rinsed in 0.1% SDS, and treated with 0.05% succinic anhydride prepared in buffer consisting of 50% 1-methyl-2-pyrrolidinone and 50% boric acid. The DNA on the slides can then be denatured in, for example, distilled water for 2 minutes at 90° C. immediately before use. Microarray solid-state samples can scanned with, for example, a laser fluorescent scanner with a computer-controlled XY stage and a microscope objective. A mixed gas, multiline laser allows sequential excitation of multiple fluorophores.

P. Open Circle Probes

An open circle probe (OCP) is a linear DNA molecule. OCPs can be any length, but preferably contain between 50 to 1000 nucleotides, more preferably between about 60 to 150 nucleotides, and most preferably between about 70 to 100 nucleotides. The OCP has a 5′ phosphate group and a 3′ hydroxyl group. This allows the ends to be ligated (to each other or to other nucleic acid ends) using a ligase, coupled, or extended in a gap-filling operation. Open circle probes can be partially double-stranded. Useful open circle probes can comprise one or more primer complement portions, one or more secondary DNA strand displacement primer matching portions, and one or more detection tag portions.

Portions of the OCP can have specific functions making the OCP useful for RCA and LM-RCA. These portions are referred to as the target probe portions, the primer complement portions, the spacer region, the secondary DNA strand displacement primer matching portions, the detection tag portions, the secondary target sequence portions, the address tag portions, and the promoter portions. These portions are analogous to similarly-named portions of ATCs and their further description elsewhere herein in the context of ATCs is applicable to the analogous portion in OCPs. The target probe portions and at least one primer complement portion are required elements of an open circle probe. The primer complement portion can be part of, for example, the spacer region. Detection tag portions, secondary target sequence portions, promoter portions, and additional primer complement portions are optional and, when present, can be part of, for example, the spacer region. Address tag portions are optional and, when present, can be part of, for example, the spacer region. The primer complement portions, and the detection tag portions, the secondary target sequence portions, the address tag portions, and the promoter portions, if present, can be non-overlapping. However, various of these portions can be partially or completely overlapping if desired. OCPs can be single-stranded but may be partially double-stranded. In use, the target probe portions of an OCP should be single-stranded so that they can interact with target sequences.

Generally, an open circle probe can be a single-stranded, linear DNA molecule comprising, from 5′ end to 3′ end, a 5′ phosphate group, a right target probe portion, a spacer region, a left target probe portion, and a 3′ hydroxyl group, with a primer complement portion present as part of the spacer region. Particularly useful open circle probes can comprise a right target probe portion, a left target probe portion, one or more primer complement portions, and a secondary DNA strand displacement primer matching portion. Those segments of the spacer region that do not correspond to a specific portion of the OCP can be arbitrarily chosen sequences. For multiply-primed RCA, a plurality of primer complement portions are required. Where random or degenerate rolling circle replication primers are used, the sequence of the primer complement portions need not either be known or of a specified sequence. The open circle probe can include at least one detection tag portion when fluorescent change probes (or other detection probes) are used for detection.

It is preferred that OCPs do not have any sequences that are self-complementary. It is considered that this condition is met if there are no complementary regions greater than six nucleotides long without a mismatch or gap. It is also preferred that OCPs containing a promoter portion do not have any sequences that resemble a transcription terminator, such as a run of eight or more thymidine nucleotides. A lack of self-complementary sequences and a lack of promoter sequences is generally not required in the case of open circle probes including, derived from, or comprising nucleic acid molecules of interest. Such features will generally not be controlled for such open circle probes.

The primer complement portions can be adjacent to the right target probe, with the right target probe portion and the primer complement portion preferably separated by three to ten nucleotides, and most preferably separated by six nucleotides, from the proximate primer complement portion. This location prevents the generation of any other spacer sequences, such as detection tags and secondary target sequences, from unligated open circle probes during DNA replication. Such an arrangement is less useful when using multiply-primed RCA. A primer complement portion can also be a part of or overlap all or a part of the target probe portions and/or any gap space sequence, if present.

The open circle probe, when ligated and replicated, gives rise to a long DNA molecule containing multiple repeats of sequences complementary to the open circle probe. This long DNA molecule is referred to herein as tandem sequences DNA (TS-DNA). TS-DNA contains sequences complementary to the target probe portions, the primer complement portion, the spacer region, and, if present on the open circle probe, the detection tag portions, the secondary target sequence portions, the address tag portions, and the promoter portion. These sequences in the TS-DNA are referred to as target sequences (which match the original target sequence), primer sequences (which match the sequence of the rolling circle replication primer), spacer sequences (complementary to the spacer region), detection tags, secondary target sequences, address tags, and promoter sequences. The TS-DNA will also have sequence complementary to the matching portion of secondary DNA strand displacement primers. This sequence in the TS-DNA is referred to as the secondary DNA strand displacement primer complement or as the primer complement.

Preferably, the promoter portion of an OCP is immediately adjacent to the left target probe and is oriented to promote transcription toward the 3′ end of the open circle probe. This orientation results in transcripts that are complementary to TS-DNA, allowing independent detection of TS-DNA and the transcripts, and prevents transcription from interfering with rolling circle replication. Open circle probes can be capable of forming an intramolecular stem structure involving one or both of the OCP's ends. Such open circle probes are referred to herein as hairpin open circle probes. Open circle probes forming intramolecular stem structures, and their use in rolling circle amplification, are described in U.S. patent application Ser. No. 09/803,713.

1. Target Probe Portions

There are two target probe portions on each OCP, one at each end of the OCP. The target probe portions can each be any length that supports specific and stable hybridization between the target probes and the target sequence. For this purpose, a length of 10 to 35 nucleotides for each target probe portion is preferred, with target probe portions 15 to 25 nucleotides long being most preferred. The target probe portion at the 3′ end of the OCP is referred to as the left target probe, and the target probe portion at the 5′ end of the OCP is referred to as the right target probe. These target probe portions are also referred to herein as left and right target probes or left and right probes. The target probe portions are complementary to a target nucleic acid sequence.

The target probe portions are complementary to the target sequence, such that upon hybridization the 5′ end of the right target probe portion and the 3′ end of the left target probe portion are base-paired to adjacent nucleotides in the target sequence, with the objective that they serve as a substrate for ligation.

In another form of open circle probe, the 5′ end and the 3′ end of the target probe portions may hybridize in such a way that they are separated by a gap space. In this case the 5′ end and the 3′ end of the OCP may only be ligated if one or more additional oligonucleotides, referred to as gap oligonucleotides, are used, or if the gap space is filled during the ligation operation. The gap oligonucleotides hybridize to the target sequence in the gap space to form a continuous probe/target hybrid. The gap space may be any length desired but is generally ten nucleotides or less. It is preferred that the gap space is between about three to ten nucleotides in length, with a gap space of four to eight nucleotides in length being most preferred. Alternatively, a gap space could be filled using a DNA polymerase during the ligation operation. When using such a gap-filling operation, a gap space of three to five nucleotides in length is most preferred. As another alternative, the gap space can be partially bridged by one or more gap oligonucleotides, with the remainder of the gap filled using DNA polymerase.

Q. DNA polymerases

DNA polymerases useful in the rolling circle replication step of the disclosed method must perform rolling circle replication of primed circular templates. Such polymerases are referred to herein as rolling circle DNA polymerases. For rolling circle replication, it is preferred that a DNA polymerase be capable of displacing the strand complementary to the template strand, termed strand displacement, and lack a 5′ to 3′ exonuclease activity. Strand displacement is necessary to result in synthesis of multiple tandem copies of ligated OCPs or ligated pairs of HCPs. A 5′ to 3′ exonuclease activity, if present, might result in the destruction of the synthesized strand. DNA polymerases for use in the disclosed method can also be highly processive, if desired. The suitability of a DNA polymerase for use in the disclosed method can be readily determined by assessing its ability to carry out rolling circle replication. Preferred rolling circle DNA polymerases are Bst DNA polymerase, VENT® DNA polymerase (Kong et al., J. Biol. Chem. 268:1965-1975 (1993)), ThermoSequenase™, delta Tts DNA polymerase, Bca DNA polymerase (Journal of Biochemistry 1 13(3):401-10, 1993 Mar.), bacteriophage φ29 DNA polymerase (U.S. Pat. Nos. 5,198,543 and 5,001,050 to Blanco et al.), phage M2 DNA polymerase (Matsumoto et al., Gene 84:247 (1989)), phage φPRD1 DNA polymerase (Jung et al., Proc. Natl. Acad. Sci. USA 84:8287 (1987)), Klenow fragment of DNA polymerase I (Jacobsen et al., Eur. J. Biochem. 45:623-627 (1974)), T5 DNA polymerase (Chatterjee et al., Gene 97:13-19 (1991)), PRD1 DNA polymerase (Zhu and Ito, Biochim. Biophys. Acta. 1219:267-276 (1994)), modified T7 DNA polymerase (Tabor and Richardson, J. Biol. Chem. 262:15330-15333 (1987); Tabor and Richardson, J. Biol. Chem. 264:6447-6458 (1989); Sequenase™ (U.S. Biochemicals)), and T4 DNA polymerase holoenzyme (Kaboord and Benkovic, Curr. Biol. 5:149-157 (1995)). More preferred are Bst DNA polymerase, VENT® DNA polymerase, ThermoSequenase™, and delta Tts DNA polymerase. Bst DNA polymerase is most preferred.

Strand displacement can be facilitated through the use of a strand displacement factor, such as helicase. It is considered that any DNA polymerase that can perform rolling circle replication in the presence of a strand displacement factor is suitable for use in the disclosed method, even if the DNA polymerase does not perform rolling circle replication in the absence of such a factor. Strand displacement factors useful in the disclosed method include BMRF1 polymerase accessory subunit (Tsurumi et al., J. Virology 67(12):7648-7653 (1993)), adenovirus DNA-binding protein (Zijderveld and van der Vliet, J. Virology 68(2):1158-1164 (1994)), herpes simplex viral protein ICP8 (Boehmer and Lehman, J. Virology 67(2):711-715 (1993); Skaliter and Lehman, Proc. Natl. Acad. Sci. USA 91(22):10665-10669 (1994)), single-stranded DNA binding proteins (SSB; Rigler and Romano, J. Biol. Chem. 270:8910-8919 (1995)), and calf thymus helicase (Siegel et al., J. Biol. Chem. 267:13629-13635 (1992)).

The ability of a polymerase to carry out rolling circle replication can be determined by using the polymerase in a rolling circle replication assay such as those described in Fire and Xu, Proc. Natl. Acad. Sci. USA 92:4641-4645 (1995) and in U.S. Pat. No. 6,143,495 (Example 1).

Another type of DNA polymerase can be used if a gap-filling synthesis step is used, such as in gap-filling LM-RCA (see U.S. Pat. No. 6,143,495, Example 3). When using a DNA polymerase to fill gaps, strand displacement by the DNA polymerase is undesirable. Such DNA polymerases are referred to herein as gap-filling DNA polymerases. Unless otherwise indicated, a DNA polymerase referred to herein without specifying it as a rolling circle DNA polymerase or a gap-filling DNA polymerase, is understood to be a rolling circle DNA polymerase and not a gap-filling DNA polymerase. Preferred gap-filling DNA polymerases are T7 DNA polymerase (Studier et al., Methods Enzymol. 185:60-89 (1990)), DEEP VENT® DNA polymerase (New England Biolabs, Beverly, Mass.), modified T7 DNA polymerase (Tabor and Richardson, J. Biol. Chem. 262:15330-15333 (1987); Tabor and Richardson, J. Biol. Chem. 264:6447-6458 (1989); Sequenase™ (U.S. Biochemicals)), and T4 DNA polymerase (Kunkel et al., Methods Enzymol. 154:367-382 (1987)). An especially preferred type of gap-filling DNA polymerase is the Thermus flavus DNA polymerase (MBR, Milwaukee, Wis.). The most preferred gap-filling DNA polymerase is the Stoffel fragment of Taq DNA polymerase (Lawyer et al., PCR Methods Appl. 2(4):275-287 (1993), King et al., J. Biol. Chem. 269(18):13061-13064 (1994)).

The ability of a polymerase to fill gaps can be determined by performing gap-filling LM-RCA. Gap-filling LM-RCA is performed with an open circle probe that forms a gap space when hybridized to the target sequence. Ligation can only occur when the gap space is filled by the DNA polymerase. If gap-filling occurs, TS-DNA can be detected, otherwise it can be concluded that the DNA polymerase, or the reaction conditions, is not useful as a gap-filling DNA polymerase.

R. DNA Ligases

Any DNA ligase is suitable for use in the disclosed method. Preferred ligases are those that preferentially form phosphodiester bonds at nicks in double-stranded DNA. That is, ligases that fail to ligate the free ends of single-stranded DNA at a significant rate are preferred. Thermostable ligases are especially preferred. Many suitable ligases are known, such as T4 DNA ligase (Davis et al., Advanced Bacterial Genetics—A Manual for Genetic Engineering (Cold Spring Harbor Laboratory, Cold Spring Harbor, N.Y., 1980)), E. coli DNA ligase (Panasnko et al., J. Biol. Chem. 253:4590-4592 (1978)), AMPLIGASE® (Kalin et al., Mutat. Res., 283(2):119-123 (1992); Winn-Deen et al., Mol Cell Probes (England) 7(3):179-186 (1993)), Taq DNA ligase (Barany, Proc. Natl. Acad. Sci. USA 88:189-193 (1991), Thermus thermophilus DNA ligase (Abbott Laboratories), Thermus scotoductus DNA ligase and Rhodothermus marinus DNA ligase (Thorbjarnardottir et al., Gene 151:177-180 (1995)). T4 DNA ligase is preferred for ligations involving RNA target sequences due to its ability to ligate DNA ends involved in DNA:RNA hybrids (Hsuih et al., Quantitative detection of HCV RNA using novel ligation-dependent polymerase chain reaction, American Association for the Study of Liver Diseases (Chicago, Ill., Nov. 3-7, 1995)).

The frequency of non-target-directed ligation catalyzed by a ligase can be determined as follows. LM-RCA is performed with an open circle probe and a gap oligonucleotide in the presence of a target sequence. Non-targeted-directed ligation products can then be detected by using an address probe specific for the open circle probe ligated without the gap oligonucleotide to capture TS-DNA from such ligated probes. Target directed ligation products can be detected by using an address probe specific for the open circle probe ligated with the gap oligonucleotide. By using a solid-state detector with regions containing each of these address probes, both target directed and non-target-directed ligation products can be detected and quantitated. The ratio of target-directed and non-target-directed TS-DNA produced provides a measure of the specificity of the ligation operation. Target-directed ligation can also be assessed as discussed in Barany (1991).

S. RNA Polymerases

Any RNA polymerase which can carry out transcription in vitro and for which promoter sequences have been identified can be used in the disclosed method to produce transcripts. Stable RNA polymerases without complex requirements are preferred. Most preferred are T7 RNA polymerase (Davanloo et al., Proc. Natl. Acad. Sci. USA 81:2035-2039 (1984)) and SP6 RNA polymerase (Butler and Chamberlin, J. Biol. Chem. 257:5772-5778 (1982)) which are highly specific for particular promoter sequences (Schenborn and Meirendorf, Nucleic Acids Research 13:6223-6236 (1985)). Other RNA polymerases with this characteristic are also preferred. Because promoter sequences are generally recognized by specific RNA polymerases, the OCP or ATC should contain a promoter sequence recognized by the RNA polymerase that is used. Numerous promoter sequences are known and any suitable RNA polymerase having an identified promoter sequence can be used. Promoter sequences for RNA polymerases can be identified using established techniques.

T. Oligonucleotide Synthesis

Half circle probes, guide oligonucleotides, amplification target circles, rolling circle replication primers, detection probes, address probes, DNA strand displacement primers, open circle probes, gap oligonucleotides and any other oligonucleotides can be synthesized using established oligonucleotide synthesis methods. Methods to produce or synthesize oligonucleotides are well known. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method. Solid phase chemical synthesis of DNA fragments is routinely performed using protected nucleoside cyanoethyl phosphoramidites (S. L. Beaucage et al. (1981) Tetrahedron Lett. 22:1859). In this approach, the 3′-hydroxyl group of an initial 5′-protected nucleoside is first covalently attached to the polymer support (R. C. Pless et al. (1975) Nucleic Acids Res. 2:773 (1975)). Synthesis of the oligonucleotide then proceeds by deprotection of the 5′-hydroxyl group of the attached nucleoside, followed by coupling of an incoming nucleoside-3′-phosphoramidite to the deprotected hydroxyl group (M. D. Matteucci et a. (1981) J. Am. Chem. Soc. 103:3185). The resulting phosphite triester is finally oxidized to a phosphorotriester to complete the internucleotide bond (R. L. Letsinger et al. (1976) J. Am. Chem. Soc. 9:3655). Alternatively, the synthesis of phosphorothioate linkages can be carried out by sulfurization of the phosphite triester. Several chemicals can be used to perform this reaction, among them 3H-1,2-benzodithiole-3-one, 1,1-dioxide (R. P. Iyer, W. Egan, J. B. Regan, and S. L. Beaucage, J. Am. Chem. Soc., 1990, 112, 1253-1254). The steps of deprotection, coupling and oxidation are repeated until an oligonucleotide of the desired length and sequence is obtained. Other methods exist to generate oligonucleotides such as the H-phosphonate method (Hall et al, (1957) J. Chem. Soc., 3291-3296) or the phosphotriester method as described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994). Other forms of oligonucleotide synthesis are described in U.S. Pat. Nos. 6,294,664 and 6,291,669.

The nucleotide sequence of an oligonucleotide is generally determined by the sequential order in which subunits of subunit blocks are added to the oligonucleotide chain during synthesis. Each round of addition can involve a different, specific nucleotide precursor, or a mixture of one or more different nucleotide precursors. In general, degenerate or random positions in an oligonucleotide can be produced by using a mixture of nucleotide precursors representing the range of nucleotides that can be present at that position. Thus, precursors for A and T can be included in the reaction for a particular position in an oligonucleotide if that position is to be degenerate for A and T. Precursors for all four nucleotides can be included for a fully degenerate or random position. Completely random oligonucleotides can be made by including all four nucleotide precursors in every round of synthesis. Degenerate oligonucleotides can also be made having different proportions of different nucleotides. Such oligonucleotides can be made, for example, by using different nucleotide precursors, in the desired proportions, in the reaction.

Many of the oligonucleotides described herein are designed to be complementary to certain portions of other oligonucleotides or nucleic acids such that stable hybrids can be formed between them. The stability of these hybrids can be calculated using known methods such as those described in Lesnick and Freier, Biochemistry 34:10807-10815 (1995), McGraw et al., Biotechniques 8:674-678 (1990), and Rychlik et al., Nucleic Acids Res. 18:6409-6412 (1990).

Oligonucleotides can be synthesized, for example, on a Perseptive Biosystems 8909 Expedite Nucleic Acid Synthesis system using standard β-cyanoethyl phosphoramidite coupling chemistry on synthesis columns (Glen Research, Sterling, Va.). Oxidation of the newly formed phosphites can be carried out using, for example, the sulfurizing reagent 3H-1,2-benzothiole-3-one-1,1-idoxide (Glen Research) or the standard oxidizing reagent after the first and second phosphoramidite addition steps. The thio-phosphitylated oligonucleotides can be deprotected, for example, using 30% ammonium hydroxide (3.0 ml) in water at 55° C. for 16 hours, concentrated in an OP 120 Savant Oligo Prep deprotection unit for 2 hours, and desalted with PD10 Sephadex columns using the protocol provided by the manufacturer.

Hexamer oligonucleotides can be synthesized on a Perseptive Biosystems 8909 Expedite Nucleic Acid Synthesis system using standard β-cyanoethyl phosphoramidite coupling chemistry on mixed dA+dC+dG+dT synthesis columns (Glen Research, Sterling, Va.). The four phosphoramidites can be mixed in equal proportions to randomize the bases at each position in the oligonucleotide. Oxidation of the newly formed phosphites can be carried out using the sulfurizing reagent 3H-1,2-benzothiole-3-one-1,1-idoxide (Glen Research) instead of the standard oxidizing reagent after the first and second phosphoramidite addition steps. The thio-phosphitylated oligonucleotides can be deprotected using 30% ammonium hydroxide (3.0 ml) in water at 55° C. for 16 hours, concentrated in an OP 120 Savant Oligo Prep deprotection unit for 2 hours, and desalted with PD10 Sephadex columns using the protocol provided by the manufacturer.

So long as their relevant function is maintained, half circle probes, guide oligonucleotides, amplification target circles, rolling circle replication primers, detection probes, address probes, DNA strand displacement primers, open circle probes, gap oligonucleotides and any other oligonucleotides can be made up of or include modified nucleotides (nucleotide analogs). Many modified nucleotides are known and can be used in oligonucleotides. A nucleotide analog is a nucleotide which contains some type of modification to either the base, sugar, or phosphate moieties. Modifications to the base moiety would include natural and synthetic modifications of A, C, G, and T/U as well as different purine or pyrimidine bases, such as uracil-5-yl, hypoxanthin-9-yl (I), and 2-aminoadenin-9-yl. A modified base includes but is not limited to 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Additional base modifications can be found for example in U.S. Pat. No. 3,687,808, Englisch et al., Angewandte Chemie, International Edition, 1991, 30, 613, and Sanghvi, Y. S., Chapter 15, Antisense Research and Applications, pages 289-302, Crooke, S. T. and Lebleu, B. ed., CRC Press, 1993. Certain nucleotide analogs, such as 5-substituted pyrimidines, 6-azapyrimidines and N-2, N-6 and O-6 substituted purines, including 2-aminopropyladenine, 5-propynyluracil and 5-propynylcytosine. 5-methylcytosine can increase the stability of duplex formation. Other modified bases are those that function as universal bases. Universal bases include 3-nitropyrrole and 5-nitroindole. Universal bases substitute for the normal bases but have no bias in base pairing. That is, universal bases can base pair with any other base. Base modifications often can be combined with for example a sugar modification, such as 2′-O-methoxyethyl, to achieve unique properties such as increased duplex stability. There are numerous United States patents such as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941, which detail and describe a range of base modifications. Each of these patents is herein incorporated by reference in its entirety, and specifically for their description of base modifications, their synthesis, their use, and their incorporation into oligonucleotides and nucleic acids.

Nucleotide analogs can also include modifications of the sugar moiety. Modifications to the sugar moiety would include natural modifications of the ribose and deoxyribose as well as synthetic modifications. Sugar modifications include but are not limited to the following modifications at the 2′ position: OH; F; O—, S—, or N-alkyl; O—, S—, or N-alkenyl; O—, S— or N-alkynyl; or O-alkyl-O-alkyl, wherein the alkyl, alkenyl and alkynyl may be substituted or unsubstituted C1 to C10, alkyl or C2 to C10 alkenyl and alkynyl. 2′ sugar modifications also include but are not limited to —O[(CH₂)n O]m CH₃, —O(CH₂)n OCH₃, —O(CH₂)n NH₂, —O(CH₂)n CH₃, —O(CH₂)n —ONH₂, and —O(CH₂)n ON[(CH₂)n CH₃)]₂, where n and m are from 1 to about 10.

Other modifications at the 2′ position include but are not limited to: C1 to C10 lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars would also include those that contain modifications at the bridging ring oxygen, such as CH₂ and S. Nucleotide sugar analogs may also have sugar mimetics such as cyclobutyl moieties in place of the pentofuranosyl sugar. There are numerous United States patents that teach the preparation of such modified sugar structures such as U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety, and specifically for their description of modified sugar structures, their synthesis, their use, and their incorporation into nucleotides, oligonucleotides and nucleic acids.

Nucleotide analogs can also be modified at the phosphate moiety. Modified phosphate moieties include but are not limited to those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkages between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts and free acid forms are also included. Numerous United States patents teach how to make and use nucleotides containing modified phosphates and include but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference its entirety, and specifically for their description of modified phosphates, their synthesis, their use, and their incorporation into nucleotides, oligonucleotides and nucleic acids.

It is understood that nucleotide analogs need only contain a single modification, but may also contain multiple modifications within one of the moieties or between different moieties.

Nucleotide substitutes are molecules having similar functional properties to nucleotides, but which do not contain a phosphate moiety, such as peptide nucleic acid (PNA). Nucleotide substitutes are molecules that will recognize and hybridize to (base pair to) complementary nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

Nucleotide substitutes are nucleotides or nucleotide analogs that have had the phosphate moiety and/or sugar moieties replaced. Nucleotide substitutes do not contain a standard phosphorus atom. Substitutes for the phosphate can be for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts. Numerous United States patents disclose how to make and use these types of phosphate replacements and include but are not limited to U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference its entirety, and specifically for their description of phosphate replacements, their synthesis, their use, and their incorporation into nucleotides, oligonucleotides and nucleic acids.

It is also understood in a nucleotide substitute that both the sugar and the phosphate moieties of the nucleotide can be replaced, by for example an amide type linkage (aminoethylglycine) (PNA). U.S. Pat. Nos. 5,539,082; 5,714,331; and 5,719,262 teach how to make and use PNA molecules, each of which is herein incorporated by reference. (See also Nielsen et al., Science 254:1497-1500 (1991)).

Oligonucleotides can be comprised of nucleotides and can be made up of different types of nucleotides or the same type of nucleotides. For example, one or more of the nucleotides in an oligonucleotide can be ribonucleotides, 2′-O-methyl ribonucleotides, or a mixture of ribonucleotides and 2′-O-methyl ribonucleotides; about 10% to about 50% of the nucleotides can be ribonucleotides, 2′-O-methyl ribonucleotides, or a mixture of ribonucleotides and 2′-O-methyl ribonucleotides; about 50% or more of the nucleotides can be ribonucleotides, 2′-O-methyl ribonucleotides, or a mixture of ribonucleotides and 2′-O-methyl ribonucleotides; or all of the nucleotides are ribonucleotides, 2′-O-methyl ribonucleotides, or a mixture of ribonucleotides and 2′-O-methyl ribonucleotides. Such oligonucleotides can be referred to as chimeric oligonucleotides.

Q. Accessory Molecules

Accessory molecules are molecules that affect the interaction of analytes and specific binding molecules or analyte capture agents. For example, accessory molecules can be molecules that compete with the binding of an analyte with an analyte capture agent or specific binding molecule. One form of competitive accessory molecules are analogs of analytes. An analog is a molecule that is similar in structure but different in competition. In this context, the analyte analog should be sufficiently similar to interact with an analyte capture agent or specific binding molecule specific for that analyte. Accessory molecules can also be molecules that aid or are necessary for interaction of an analyte and a specific binding molecule or analyte capture agent. Such accessory molecules are referred to herein as analyte binding co-factors.

In one form of the disclosed method, accessory molecules can be compounds that are to be tested for their effect on analyte binding. For example, the disclosed method can be used to screen for competitors (or binding co-factors) of an analyte interaction with a specific binding molecule or analyte capture agent. If an accessory molecule affects interaction of the analyte, the results of pmRCA will change since the association of the binding guide conjugate to the analyte (or of the analyte capture agent to the analyte) will be lost or gained.

In use, the accessory molecules need not be absolutely pure. The accessory molecules preferably are at least 20% pure, more preferably at least 50% pure, more preferably at least 80% pure, and more preferably at least 90% pure.

U. Analyte Capture Agents

An analyte capture agent is any compound that can interact with an analyte and allow the analyte to be immobilized or separated from other compounds and analytes. An analyte capture agent includes an analyte interaction portion. Analyte capture agents can also include a capture portion. Analyte capture agents without a capture portion preferably are immobilized on a solid support. The analyte interaction portion of an analyte capture agent is a molecule that interacts specifically with a particular molecule or moiety. The molecule or moiety that interacts specifically with an analyte interaction portion can be an analyte or another molecule that serves as an intermediate in the interaction between the analyte interaction portion and the analyte. It is to be understood that the tern analyte refers to both separate molecules and to portions of molecules, such as an epitope of a protein, that interacts specifically with an analyte interaction portion. Antibodies, either member of a receptor/ligand pair, and other molecules with specific binding affinities are examples of molecules that can be used as an analyte interaction portion of an analyte capture agent. The specific binding portion of an analyte capture agent can also be any compound or composition with which an analyte can interact, such as peptides. An analyte capture agent that interacts specifically with a particular analyte is said to be specific for that analyte. For example, an analyte capture agent with an analyte interaction portion that is an antibody that binds to a particular antigen is said to be specific for that antigen. The antigen is the analyte.

Examples of molecules useful as the analyte interaction portion of analyte capture agents are antibodies, such as crude (serum) antibodies, purified antibodies, monoclonal antibodies, polyclonal antibodies, synthetic antibodies, antibody fragments (for example, Fab fragments); antibody interacting agents, such as protein A, carbohydrate binding proteins, and other interactants; protein interactants (for example avidin and its derivatives); peptides; and small chemical entities, such as enzyme substrates, cofactors, metal ions/chelates, and haptens. Antibodies may be modified or chemically treated to optimize binding to surfaces and/or targets.

Antibodies useful as the analyte interaction portion of analyte capture agents, can be obtained commercially or produced using well-established methods. For example, Johnstone and Thorpe, on pages 30-85, describe general methods useful for producing both polyclonal and monoclonal antibodies. The entire book describes many general techniques and principles for the use of antibodies in assay systems.

The capture portion of an analyte capture agent is any compound that can be associated with another compound. Preferably, a capture portion is a compound, such as a ligand or hapten, that binds to or interacts with another compound, such as ligand-binding molecule or an antibody. It is also preferred that such interaction between the capture portion and the capturing component be a specific interaction, such as between a hapten and an antibody or a ligand and a ligand-binding molecule. Examples of haptens include biotin, FITC, digoxigenin, and dinitrophenol. The capture portion can be used to separate compounds or complexes associated with the analyte capture agent from those that do not.

Capturing analytes or analyte capture agents on a substrate may be accomplished in several ways. In one embodiment, capture docks are adhered or coupled to the substrate. Capture docks are compounds or moieties that mediate adherence of an analyte by binding to, or interacting with, the capture portion on an analyte capture agent (with which the analyte is, or will be, associated). Capture docks immobilized on a substrate allow capture of the analyte on the substrate. Such capture provides a convenient means of washing away reaction components that might interfere with subsequent steps. Alternatively, analyte capture agents can be directly immobilized on a substrate. In this case, the analyte capture agent need not have a capture portion.

In one embodiment, the analyte capture agent or capture dock to be immobilized is an anti-hybrid antibody. Methods for immobilizing antibodies and other proteins to substrates are well established. Immobilization can be accomplished by attachment, for example, to aminated surfaces, carboxylated surfaces or hydroxylated surfaces using standard immobilization chemistries. Examples of attachment agents are cyanogen bromide, succinimide, aldehydes, tosyl chloride, avidin-biotin, photocrosslinkable agents, epoxides and maleimides. A preferred attachment agent is a heterobifunctional cross-linking agent such as N-[γ-maleimidobutyryloxy]succinimide ester (GMBS). These and other attachment agents, as well as methods for their use in attachment, are described in Protein immobilization: fundamentals and applications, Richard F. Taylor, ed. (M. Dekker, New York, 1991), Johnstone and Thorpe, Immunochemistry In Practice (Blackwell Scientific Publications, Oxford, England, 1987) pages 209-216 and 241-242, and Immobilized Affinity Ligands, Craig T. Hermanson et al., eds. (Academic Press, New York, 1992). Antibodies can be attached to a substrate by chemically cross-linking a free amino group on the antibody to reactive side groups present within the substrate. For example, antibodies may be chemically cross-linked to a substrate that contains free amino, carboxyl, or sulfur groups using glutaraldehyde, carbodiimides, or heterobifunctional agents such as GMBS as cross-linkers. In this method, aqueous solutions containing free antibodies are incubated with the solid-state substrate in the presence of glutaraldehyde or carbodiimide. For crosslinking with glutaraldehyde the reactants can be incubated with 2% glutaraldehyde by volume in a buffered solution such as 0.1 M sodium cacodylate at pH 7.4. Other standard immobilization chemistries are known by those of skill in the art.

One useful form of analyte capture agents are peptides. When various peptides are immobilized in an array, they can be used as “bait” for analytes. For example, an array of different peptides can be used to access whether a sample has analytes that interact with any of the peptides. Comparisons of different samples can be made by, for example, noting differences in the peptides to which analytes in the different samples become associated. In another form of the disclosed method, an array of analyte capture agents specific for analytes of interest can be used to access the presence of a whole suite of analytes in a sample.

V. Nucleic Acid Molecules

The disclosed method can involve use of nucleic acid molecules and nucleic acid sequences as nucleic acid molecules of interest and as a source for target sequences and nucleic acid sequences of interest. Nucleic acid molecules of interest can be, or can be used in, amplification target circles. As used herein, unless the context indicates otherwise, the term nucleic acid molecule refers to both actual molecules and to nucleic acid sequences that are part of a larger nucleic acid molecule.

Nucleic acid molecule and sequences can be from any nucleic acid sample of interest. The source, identity, and preparation of many such nucleic acid samples are known. It is useful if nucleic acid samples known or identified for use in amplification or detection methods are used for the method described herein. The nucleic acid sample can be, for example, a nucleic acid sample from one or more cells, tissue, or bodily fluids such as blood, urine, semen, lymphatic fluid, cerebrospinal fluid, or amniotic fluid, or other biological samples, such as tissue culture cells, buccal swabs, mouthwash, stool, tissues slices, biopsy aspiration, and archeological samples such as bone or mummified tissue. Types of useful nucleic acid samples include blood samples, urine samples, semen samples, lymphatic fluid samples, cerebrospinal fluid samples, amniotic fluid samples, biopsy samples, needle aspiration biopsy samples, cancer samples, tumor samples, tissue samples, cell samples, cell lysate samples, a crude cell lysate samples, forensic samples, archeological samples, infection samples, nosocomial infection samples, production samples, drug preparation samples, biological molecule production samples, protein preparation samples, lipid preparation samples, and/or carbohydrate preparation samples.

Nucleic acid molecules and nucleic acid sequences that have or are sequences complementary to target probe portions of an open circle probe or the guide complement portions of a half circle probe are also referred to as target molecules and target sequences. Examples of target molecules, target sequences, or sources of target sequences are mRNA molecules and cDNA molecules, although any nucleic acid molecule or sequence can be used in the disclosed compositions and method. Target sequences and guide sequences, which can be the object of amplification, can be any nucleic acid. Target sequences and guide sequences can include multiple nucleic acid molecules, such as in the case of mRNA amplification, multiple sites in a nucleic acid molecule, or a single region of a nucleic acid molecule. For example, target sequences and guide sequences can be mRNA and cDNA.

W. Nucleic Acid Samples

Nucleic acid samples can be derived from any source that has, or is suspected of having, nucleic acids. A nucleic acid sample is the source of nucleic acid molecules and nucleic acid sequences. Nucleic acid sample can contain, for example, a target nucleic acid, for example a specific mRNA or pool of mRNA molecules. The nucleic acid sample can contain RNA or DNA or both. The nucleic acid sample in certain embodiments can also include chemically synthesized nucleic acids. The nucleic acid sample can include any nucleotide, nucleotide analog, nucleotide substitute or nucleotide conjugate.

The nucleic acid sample can be, for example, a nucleic acid sample from one or more cells, tissue, or bodily fluids such as blood, urine, semen, lymphatic fluid, cerebrospinal fluid, or amniotic fluid, or other biological samples, such as tissue culture cells, buccal swabs, mouthwash, stool, tissues slices, biopsy aspiration, and archeological samples such as bone or mummified tissue. Types of useful nucleic acid samples include blood samples, urine samples, semen samples, lymphatic fluid samples, cerebrospinal fluid samples, amniotic fluid samples, biopsy samples, needle aspiration biopsy samples, cancer samples, tumor samples, tissue samples, cell samples, cell lysate samples, crude cell lysate samples, forensic samples, archeological samples, infection samples, nosocomial infection samples, production samples, drug preparation samples, biological molecule production samples, protein preparation samples, lipid preparation samples, and/or carbohydrate preparation samples.

X. Target Fingerprints

The disclosed method can identify, produce, or generate information about, for example, the presence, absence, amount, level, or condition of target molecules, target sequences, and analytes. Such information can be referred to as a target fingerprint. Target fingerprints can be used for any purpose and in any other method as appropriate for the type and quality of information involved. For example, the amount of an analyte in one sample can be compared to the amount of the same analyte in a different sample.

Informational content of, or derived from, the disclosed method can also be stored. Such information can be stored, for example, in or as computer readable media. Information generated in the disclosed method can be combined with information obtained or generated from any other source. The informational nature of target fingerprints produced using the disclosed method lends itself to combination and/or analysis using known bioinformatics systems and methods.

Target fingerprints of samples can be compared to similar target fingerprints derived from any other sample to detect similarities and differences in the samples (which is indicative of similarities and differences in the target molecules in the samples). For example, a target fingerprint of a first sample can be compared to a target fingerprint of a sample from the same type of organism as the first sample, a sample from the same type of tissue as the first sample, a sample from the same organism as the first sample, a sample obtained from the same source but at time different from that of the first sample, a sample from an organism different from that of the first sample, a sample from a type of tissue different from that of the first sample, a sample from a strain of organism different from that of the first sample, a sample from a species of organism different from that of the first sample, or a sample from a type of organism different from that of the first sample.

The same type of tissue is tissue of the same type such as liver tissue, muscle tissue, or skin (which may be from the same or a different organism or type of organism). The same organism refers to the same individual, animal, or cell. For example, two samples taken from a patient are from the same organism. The same source is similar but broader, referring to samples from, for example, the same organism, the same tissue from the same organism, the same DNA molecule, or the same DNA library. Samples from the same source that are to be compared can be collected at different times (thus allowing for potential changes over time to be detected). This is especially useful when the effect of a treatment or change in condition is to be assessed. Samples from the same source that have undergone different treatments can also be collected and compared using the disclosed method. A different organism refers to a different individual organism, such as a different patient or a different individual animal. Different organism includes a different organism of the same type or organisms of different types. A different type of organism refers to organisms of different types such as a dog and cat, a human and a mouse, or E. coli and Salmonella. A different type of tissue refers to tissues of different types such as liver and kidney, or skin and brain. A different strain or species of organism refers to organisms differing in their species or strain designation as those terms are understood in the art.

Y. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example, disclosed are kits for proximity-mediatedrolling circle amplification, the kit comprising a plurality of binding guide conjugates and a plurality of half circle probes. The kits can also contain one or more rolling circle replication primers and one or more fluorescent change probes. The kits also can contain DNA polymerase, amplification target circles, nucleotides, buffers, ligase, open circle probes, linkers, circularization sequences, or a combination.

Z. Mixtures

Disclosed are mixtures formed by performing or preparing to perform the disclosed method. For example, disclosed are mixtures comprising one or more binding guide conjugates and one or more half circle probes; one or more binding guide conjugates and one or more guide oligonucleotides; one or more binding guide conjugates, one or more amplification target circles, and one or more rolling circle replication primers; one or more binding guide conjugates, one or more amplification target circles, one or more rolling circle replication primers, and one or more fluorescent change probes; one or more binding guide conjugates, tandem sequence DNA, one or more amplification target circles, one or more rolling circle replication primers, and one or more fluorescent change probes; one or more binding guide conjugates, DNA polymerase, one or more amplification target circles, one or more rolling circle replication primers, and one or more fluorescent change probes; one or more binding guide conjugates, DNA polymerase, tandem sequence DNA, one or more amplification target circles, one or more rolling circle replication primers, and one or more fluorescent change probes; secondary tandem sequence DNA, one or more amplification target circles, one or more rolling circle replication primers, and one or more fluorescent change probes; one or more binding guide conjugates, tandem sequence DNA, secondary tandem sequence DNA, one or more amplification target circles, one or more rolling circle replication primers, and one or more fluorescent change probes; one or more binding guide conjugates, DNA polymerase, tandem sequence DNA, secondary tandem sequence DNA, one or more amplification target circles, one or more rolling circle replication primers, and one or more fluorescent change probes; one or more binding guide conjugates, one or more amplification target circles and one or more fluorescent change rolling circle replication primers; tandem sequence DNA, one or more amplification target circles, and one or more fluorescent change rolling circle replication primers; one or more binding guide conjugates, DNA polymerase, one or more amplification target circles, and one or more fluorescent change rolling circle replication primers; one or more binding guide conjugates, DNA polymerase, tandem sequence DNA, one or more amplification target circles, and one or more fluorescent change rolling circle replication primers; one or more binding guide conjugates, secondary tandem sequence DNA, one or more amplification target circles, and one or more fluorescent change rolling circle replication primers; one or more binding guide conjugates, tandem sequence DNA, secondary tandem sequence DNA, one or more amplification target circles, and one or more fluorescent change rolling circle replication primers; or one or more binding guide conjugates, DNA polymerase, tandem sequence DNA, secondary tandem sequence DNA, one or more amplification target circles, and one or more fluorescent change rolling circle replication primers.

Whenever the method involves mixing or bringing into contact compositions or components or reagents, performing the method creates a number of different mixtures. For example, if the method includes 3 mixing steps, after each one of these steps a unique mixture is formed if the steps are performed separately. In addition, a mixture is formed at the completion of all of the steps regardless of how the steps were performed. The present disclosure contemplates these mixtures, obtained by the performance of the disclosed methods as well as mixtures containing any disclosed reagent, composition, or component, for example, disclosed herein.

AA. Systems

Disclosed are systems useful for performing, or aiding in the performance of, the disclosed method. Systems generally comprise combinations of articles of manufacture such as structures, machines, devices, and the like, and compositions, compounds, materials, and the like. Such combinations that are disclosed or that are apparent from the disclosure are contemplated. For example, disclosed and contemplated are systems comprising solid supports and half circle probes, binding guide conjugates, rolling circle replication primers, amplification target circles, fluorescent change probes, or a combination.

BB. Data Structures and Computer Control

Disclosed are data structures used in, generated by, or generated from, the disclosed method. Data structures generally are any form of data, information, and/or objects collected, organized, stored, and/or embodied in a composition or medium. A target fingerprint stored in electronic form, such as in RAM or on a storage disk, is a type of data structure.

The disclosed method, or any part thereof or preparation therefor, can be controlled, managed, or otherwise assisted by computer control. Such computer control can be accomplished by a computer controlled process or method, can use and/or generate data structures, and can use a computer program. Such computer control, computer controlled processes, data structures, and computer programs are contemplated and should be understood to be disclosed herein.

### Uses

The disclosed method and compositions are applicable to numerous areas including, but not limited to, detection of analytes, analysis of nucleic acids present in cells (for example, analysis of genomic DNA in cells), disease detection, mutation detection, gene discovery, gene mapping (molecular haplotyping), and agricultural research. Particularly useful is plate based immunoassays, such as monoplex and multiplex assays. The disclosed method is also useful for detection of analytes directly in solution. Other uses include, for example, detection of nucleic acids in cells and on genomic DNA arrays; molecular haplotyping; mutation detection; detection of inherited diseases such as cystic fibrosis, muscular dystrophy, diabetes, hemophilia, sickle cell anemia; assessment of predisposition for cancers such as prostate cancer, breast cancer, lung cancer, colon cancer, ovarian cancer, testicular cancer, pancreatic cancer.

### Method

Disclosed are compositions and methods for proximity-mediated rolling circle amplification and for real-time detection of proximity-mediated rolling circle amplification products. Rolling circle amplification (RCA) refers to nucleic acid amplification reactions involving replication of a circular nucleic acid template (referred to as an amplification target circle; ATC) to form a long strand (referred to as tandem sequence DNA; TS-DNA) with tandem repeats of the sequence complementary to the circular template. Proximity-mediated rolling circle amplification is a type of rolling circle amplification whereby the process is mediated in someway by the proximity or spatial relationship of various molecules involved in the amplification. Generally, proximity-mediated RCA can be accomplished by using the proximity of certain molecules or moieties as a condition that affects the formation of an amplification target circle.

In some forms, the disclosed method involves association of binding guide conjugates to the same analyte or to two analytes in close proximity. This brings the binding guide conjugates into close proximity. In these forms of the method, the binding guide conjugates comprise a specific binding molecule and a guide oligonucleotide. The guide oligonucleotides are complementary to guide complement portions on half circle probes. The complementary sequences between the guide oligonucleotides and half circle probes generally can be chosen such that two half circle probes each are complementary to both of the guide oligonucleotides in the binding guide conjugates that are in close proximity. In particular, this arrangement can allow both ends of each half circle probe to hybridize adjacent to an end of the other half circle probe and to be ligated together to form a circular nucleic acid molecule comprising the two half circle probes. In the method, this circularization generally will take place only when the binding guide conjugates are bound to analytes that bring them into close proximity. In an assay, if there is no analyte present, the binding guide conjugates will not be brought into close proximity and the half circle probes will not be circularized. The amount of circularized half circle probes formed can be a measure of the amount of analyte in a sample.

In other forms of the disclosed method, binding guide conjugates comprise a half circle probe and a guide oligonucleotide. The binding guide conjugates are associated with the same analyte or to two analytes in close proximity, bringing the binding guide conjugates into close proximity. The guide complement portions on the half circle probes are complementary to guide oligonucleotides. The complementary sequences between the guide oligonucleotides and half circle probes generally can be chosen such that the guide oligonucleotides each are complementary to both half circle probes in the binding guide conjugates that are in close proximity. In particular, this arrangement can allow both ends of each half circle probe to hybridize adjacent to an end of the other half circle probe and to be ligated together to form a circular nucleic acid molecule comprising the two half circle probes. In the method, this circularization generally will take place only when the binding guide conjugates are bound to analytes that bring them into close proximity. In an assay, if there is no analyte present, the binding guide conjugates will not be brought into close proximity and the half circle probes will not be circularized. The amount of circularized half circle probes formed can be a measure of the amount of analyte in a sample.

These forms of the disclosed method also can be performed in a competitive assay mode. In this mode, the assay is carried out in the presence of analyte(s) immobilized on a solid support. The immobilized analytes are the same as the analytes to be assayed. This analyte competes for analyte present in a sample being assayed. In the absence of analyte in the sample, the specific binding molecules of the binding guide conjugates can associate with the immobilized analyte(s), thus bringing the binding guide conjugates and their guide oligonucleotides into close proximity. This allows circularization of half circle probes as described above. In the presence of analyte in the sample, the specific binding molecules of the binding guide conjugates can associate with analyte in the sample thus keeping the binding guide conjugates from associating with the analyte in the second binding guide conjugate. As a result, the binding guide conjugates generally will bind to different antigens in solution and will not be in close proximity, which prevents the half circle probes from being circularized. The effect of any binding guide conjugates that do bind to the same antigen (bringing them into close proximity) can be eliminated by washing to remove materials not associated with the solid support. In an assay, the competition between sample analytes and analytes in binding guide conjugates can result in either elimination of circle formation or a reduction of circle formation, depending on the relative concentrations of the second binding guide conjugate and analyte in the sample. Thus, the amount of circularized half circle probes formed can be a measure of the concentration of analyte in a sample. This competitive assay mode can also be performed with the competitive analyte free in solution rather than immobilized. The analyte used to compete with analyte in a sample can be referred to as a test analyte.

In other forms of the disclosed method, two forms of binding guide conjugate are used. One form of binding guide conjugate comprises a specific binding molecule and a guide oligonucleotide. The other form of binding guide conjugate, which can be referred to as a binding guide analyte, comprises an analyte and a guide oligonucleotide. The specific binding molecule of the first binding guide conjugate is specific for the analyte in the second binding guide conjugate. These forms of binding guide conjugates can be used to detect the same type of analyte in a sample by a competitive mechanism. In the absence of analyte in the sample, the specific binding molecule of the first binding guide conjugate can associate with the analyte in the second binding guide conjugate, thus bringing the binding guide conjugates and their guide oligonucleotides into close proximity. The guide oligonucleotides are complementary to guide complement portions on half circle probes. The complementary sequences between the guide oligonucleotides and half circle probes generally can be chosen such that two half circle probes each are complementary to both of the guide oligonucleotides in the binding guide conjugates that are in close proximity. In particular, this arrangement can allow both ends of each half circle probe to hybridize adjacent to an end of the other half circle probe and to be ligated together to form a circular nucleic acid molecule comprising the two half circle probes. This circularization generally will take place only when the first binding guide conjugate is bound to the analyte in the second binding guide conjugate (which brings the binding guide conjugates into close proximity.

In the presence of analyte in the sample, the specific binding molecule of the first binding guide conjugate can associate with analyte in the sample thus keeping the first binding guide conjugate from associating with the analyte in the second binding guide conjugate. As a result, the first and second binding guide conjugates will not be in close proximity and the half circle probes will not be circularized. In an assay, the competition between sample analytes and analytes in binding guide conjugates can result in either elimination of circle formation or a reduction of circle formation, depending on the relative concentrations of the second binding guide conjugate and analyte in the sample. Thus, the amount of circularized half circle probes formed can be a measure of the concentration of analyte in a sample. These forms of the method can also be performed with binding guide conjugates having half circle probes instead of guide oligonucleotides.

Both the first and second binding guide conjugates can be used free in solution. Alternatively, either the first binding guide conjugate or the second binding guide conjugate can be immobilized on a solid support. In this case, analyte in a sample prevents or reduces association of the first binding guide conjugates with the solid support (via the specific binding molecule of an immobilized first binding guide conjugate) or prevents or reduces association of the second binding guide conjugate with the solid support (via the analyte of the immobilized second binding guide conjugate).

The disclosed methods can involve components that are all in solution or with some components immobilized. Some forms of the method performed all in solution can be performed as homogeneous assays where the assay components and the samples can be mixed and the assay performed without washing, separation or purification. this can greatly simplify the method resulting in a highly efficient assay suitable for high throughput applications.

The analytes can be any molecule or moiety of interest. For example, proteins and peptides, nucleic acids, and other biological molecules are useful analytes. Nucleic acids can be detected using binding guide conjugates that use, for example, an oligonucleotide for the specific binding molecule. Because both the specific binding molecule and the guide oligonucleotide can be oligonucleotides in such binding guide conjugates, the guide oligonucleotides can be, for example, a single oligonucleotide. Binding guide conjugates for detection of nucleic acids can be brought into close proximity by, for example, choosing guide oligonucleotide sequence that is complementary to sequences in a nucleic acid molecule of interest that close together. Nucleic acids can also be detected using binding guide conjugates having specific binding molecules that bind to haptens or other labeling moieties that can be incorporated into or attached to nucleic acid molecules. A pair of binding guide conjugates can also be directed to different types of binding targets in nucleic acid molecules. For example, one binding guide conjugate can have a nucleic acid probe as the specific binding molecule and the other can have a specific binding molecule that binds to a hapten or label. Binding guide conjugates in a pair can be directed to different types and classes of analytes. All that is required for detection using the disclosed methods is for the analytes to be in close proximity.

Also disclosed is a method for real-time detection of analytes using proximity mediated rolling circle amplification products. Real-time detection is detection that takes place during the amplification reaction or operation. Generally, such detection can be accomplished by detecting amplification product at one or more discrete times during amplification, continuously during all or one or more portions of the amplification, or a combination of discrete times and continuous detection. Real-time detection can be aided by the use of labels or moieties that embody or produce a detectable signal that can be detected without disrupting the amplification reaction or operation. Fluorescent labels are an example of useful labels for real-time detection. A particularly useful means of obtaining real-time detection is the use of fluorescent change probes and/or primers in the amplification operation. With suitably designed fluorescent change probes and primers, fluorescent signals can be generated as amplification proceeds. In most such cases, the fluorescent signals will be in proportion to the amount of amplification product and/or amount of target sequence or target molecule.

In some forms, the disclosed method involves rolling circle amplification and real-time detection of amplification products where amplification includes multiply-primed rolling circle amplification (MPRCA). Rolling circle amplification (RCA) refers to nucleic acid amplification reactions involving replication of a circular nucleic acid template to form a long strand with tandem repeats of the sequence complementary to the circular template. Rolling circle replication can be primed at one or more sites on the circular template. Multiply-primed RCA refers to RCA where replication is primed at a plurality of sites on the circular template. Multiply-primed RCA increases the sensitivity of singly-primed rolling circle amplification. Rolling circle amplification refers both to rolling circle replication and to processes involving both rolling circle replication and additional forms of amplification (such as replication of tandem sequence DNA).

Multiply-primed RCA can be performed using a single primer (which hybridizes to multiple sites on the amplification target circle) or multiple primers (each of which can hybridize to a single site on the amplification target circle or multiple sites on the amplification target circle). Multiple priming (as occurs in MPRCA) can increase the yield of amplified product from RCA. Primers anneal to multiple locations on the circular template and a product of extension by polymerase is initiated from each location. In this way, multiple extensions are achieved simultaneously from a single amplification target circle.

Multiple priming can be achieved in several different ways. For example, two or more specific primers that anneal to different sequences on the circular template can be used, one or more specific primers that each anneals to a sequence repeated at two or more separate locations on the circular template can be used, a combination of primers that each anneal to a different sequence on the circular template or to a sequence repeated at two or more separate locations on the circular templates can be used, one or more random or degenerate primers, which can anneal to many locations on the circle, can be used, or a combination of such primers can be used.

Also disclosed are methods of producing small circular single-stranded nucleic acid molecules, such as amplification target circles. The method generally involves hybridization of two or more half circle probes with two or more guide oligonucleotides in close proximity and ligation of the ends of the half circle probes. The guide oligonucleotides are complementary to guide complement portions on the half circle probes. The complementary sequences between the guide oligonucleotides and half circle probes generally can be chosen such that two half circle probes each are complementary to both of the guide oligonucleotides in the binding guide conjugates that are in close proximity. In particular, this arrangement can allow both ends of each half circle probe to hybridize adjacent to an end of the other half circle probe and to be ligated together to form a circular nucleic acid molecule comprising the two half circle probes. The guide oligonucleotides for production of circular nucleic acid molecules can be brought into close proximity in any suitable manner such as those disclosed elsewhere herein. The guide oligonucleotides also can be coupled together, part of the same molecule, or parts of a single oligonucleotide (which has the effect of placing them in close proximity). Prior methods of small circle production generally involve ligation of the two ends of a single oligonucleotide. This requires synthesis of a full length oligonucleotide. In the disclosed method, two or more shorter oligonucleotides (that is, half circle probes) can be used. Despite involving ligation of multiple molecules, the disclosed method is efficient because the guide oligonucleotides used to mediate ligation and circularization of the half circle probes are in close proximity which brings the multiple ends of the half circle probes into ligatable proximity.

A. Use of Binding Guide Conjugates

A useful form of the disclosed method uses binding guide conjugates having half circle probes or guide oligonucleotides as the oligonucleotide portion. When a pair of binding guide conjugates is in close proximity, the hybridization of a pair of half circle probes to a pair of guide oligonucleotides is facilitated. The matched pair of half circle probes can then be ligated at each end to produce an amplification target circle that can be amplified as described herein.

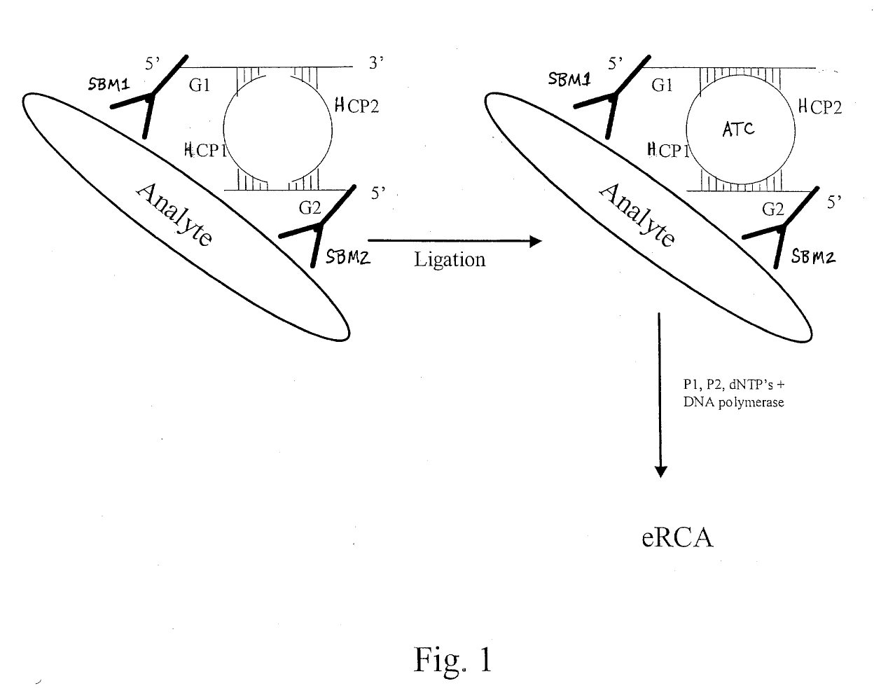

The affinity portion of the binding guide conjugate is a specific binding molecule specific for a target molecule of interest, such as proteins or peptides. The binding guide conjugate is associated with a target molecule or analyte by an interaction between the specific binding molecule and the target molecule, and detection of this interaction is facilitated by proximity-mediated rolling circle amplification. The specific binding molecules can be antibody molecules that are chosen for a particular assay, such that each can bind to adjacent, closely spaced epitopes on the intended target. Specific binding antibodies can be, for example, the same polyclonal antibodies or two monoclonal antibodies with non-overlapping epitopes. Alternatively, if the target contains multiple epitopes, a single monoclonal antibody can be the specific binding molecule for each binding guide conjugate. The target can be the analyte itself or, for example, biotins on a biotinylated detector antibody. Depending on the format, the analyte or biotinylated detector may be part of an immuno-sandwich. The oligonucleotide portions of binding guide conjugates (e.g., the guide oligonucleotides or a half circle probes) will be attached to their respective antibodies in such a way as to give the ligation substrate depicted in FIG. 1 when annealed to a pair of half circle probes.

A binding guide conjugate that contains a guide oligonucleotide as its oligonucleotide portion can be associated with a target molecule or analyte. Half circle probes can hybridize to the guide oligonucleotide of a binding guide conjugate. When a binding guide conjugate that is associated with a target molecule or analyte is in close proximity to another binding guide conjugate that is associated with a target molecule or analyte, half circle probes can hybridize to the guide oligonucleotides of both binding guide conjugates. A pair of half circle probes that are hybridized to two different guide oligonucleotides on two different binding guide conjugates (e.g., a matched pair of HCPs) can be ligated together (e.g., double ligation) to form an ATC. That is, the guide complement portions on one end of each half circle probe in a matched pair of half circle probes can hybridize to the guide oligonucleotide of one binding guide conjugate and the other end of each half circle probe in a matched pair of half circle probes can hybridize to the guide oligonucleotide of another binding guide conjugate that is in close proximity to the other binding guide conjugate. Then the two ends of one half circle probe (which are hybridized to the guide oligonucleotides of two separate binding guide conjugates) can be ligated to the two ends of the other half circle probe hybridized to the same guide oligonucleotides (using gap oligonucleotide, if desired.) FIG. 1

The ligation operations of the pair of half circle probes can take place sequentially or simultaneously. That is, a pair of half circle probes that are hybridized at one end to the same guide oligonucleotide of a binding guide conjugate can be ligated together, and then the other ends of the half circle probes can be hybridized to the guide oligonucleotide of another binding guide conjugate in close proximity to the first, followed by a second, subsequent ligation. Or a pair of half circle probes that are hybridize to two different guide oligonucleotides (a matched pair of HCPs) can be ligated at both ends simultaneously. After a pair of half circle probes have been ligated together at both ends (either sequentially or simultaneously), the resulting ATC that is formed can be amplified. The resulting TS-DNA can remain associated with the ligated pair of half circle probes, thus associating the TS-DNA to the site of the target molecule.

Unbound binding guide conjugates (e.g., binding guide conjugates that are not associated with a target molecule or analyte) can be removed by washing. If washing is not done, the association of the binding guide conjugates with an analyte and formation of an amplification target circle can be combined by using a high-temperature polymerase, such as Bst. In this case, following a short low temp incubation to allow ligation of a pair of half circle probes, the temperature can be raised to allow efficient RCA and to inactivate the ligase (although ligase inactivation need not be required). A guide oligonucleotide can be extended by the polymerase, eliminating the need for a rolling circle replication primer (P1 in FIG. 1) (the 3′ ends of the guide oligonucleotide will be complementary to the half circle probes). Omission of rolling circle replication primer and secondary DNA strand displacement primer (P1 and P2) can give rise to a linear RCA physically attached to the target molecule or analyte. A lack of a requirement for a wash step may facilitate the development of homogeneous, ultra-sensitive eRCA based immunoassays.

Addition of a rolling circle replication primer and secondary DNA strand displacement primer (P1 and P2 in FIG. 1), dNTP's, and an appropriate polymerase will result in exponential RCA (ERCA) (FIG. 1). Omission of secondary DNA strand displacement primer (P2) should result in linear RCA. A two step RCA reaction in which only polymerase and dNTP's are added first and then at a later point secondary DNA strand displacement primers (P1 and P2), may help to eliminate certain artifacts associated with ERCA. In the first step a large amount of linear substrate would be produced thus favoring the correct reaction upon addition of guide sequence P1 and P2 and suppressing side reactions.

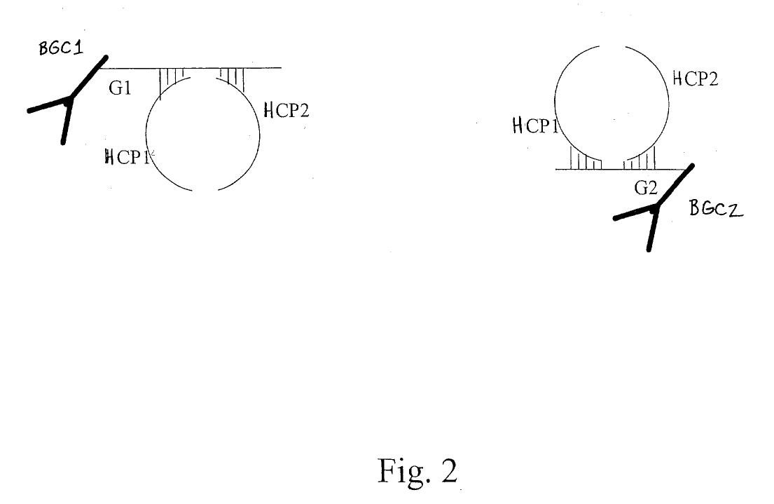

In the presence of excess half circle probes (HCP1 and HCP2 in FIG. 2), unbound and/or non-specifically bound guide conjugates can be blocked in a form not suitable for further annealing or circle formation (FIG. 2). Only when both guide conjugates are in close proximity would both half circle probes be likely to anneal and be circularized, as in FIG. 1. Blocking in unbound binding guide conjugates will further reduce background signals and facilitate the development of homogeneous assays.

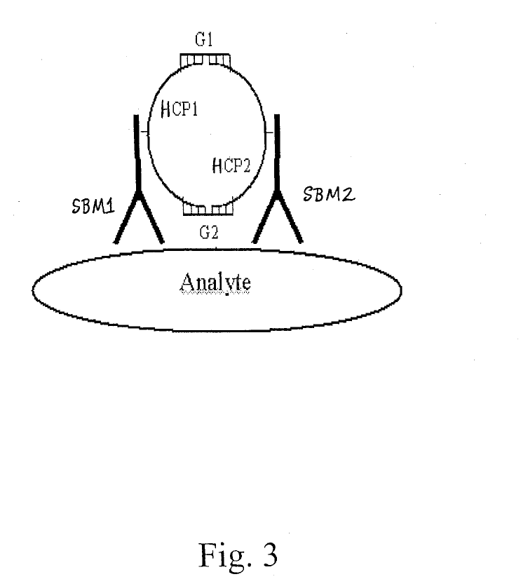

Alternatively, a binding guide conjugate that contains a half circle probe as its oligonucleotide portion can be associated with a target molecule or analyte and the associated half circle probe can be ligated to another half circle probe that is associated with another binding guide conjugate that is in close proximity by the addition of a pair of guide oligonucleotides that are complementary to the guide complement portions of the two half circle probes. The resulting amplification target circle can be amplified to detect the target molecule (FIG. 3).

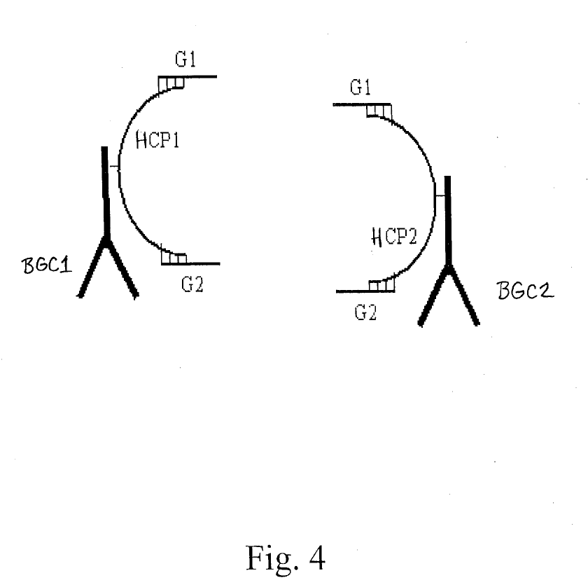

In the presence of excess guide oligonucleotides (G1 and G2 in FIG. 4) unbound and/or non-specifically bound binding guide conjugates can be blocked in a form not suitable for either ligation or further annealing (FIG. 4). Blocking in this manner does not lead to formation of open circle probes by single ligation of half circle probes.

The addition of half circle probes or guide oligonucleotides to binding guide conjugates can be sequential or simultaneous (as part of a master mix). If binding guide conjugates with guide oligonucleotides are used, sequential addition of the half circle probes can possibly reduce ligation events which don't lead to ATC formation. Similarly, if binding guide conjugates with half circle probes are used, sequential addition of guide oligonucleotides can possibly reduce ligation events which don't lead to ATC formation. Such events are possible due to multiple guides oligonucleotides or half circle probes on each binding guide conjugate. For example, annealing of a first half circle probe (HCP1 in FIG. 1) can bring two adjacent binding guide conjugates into even closer proximity thus increasing the likelihood that a second half circle probe (HCP2 in FIG. 1),when added, will anneal to the same two guide oligonucleotides thus giving rise to an amplification target circle following ligation. Thus, when one end of a half circle probe anneals to a guide oligonucleotide, the other end is highly likely to anneal to another guide oligonucleotide that is in close proximity due to the increase in the effective concentration as a result of adjacent binding guide conjugates. The close proximity of the binding guide conjugates needed for the efficient ligation of the half circle probes is unlikely to occur if the binding guide conjugates bind non-specifically to their target. This together with the lack of requirement for preformed circles should greatly reduce the background in eRCA based immunoassays. Similarly, annealing of a first guide oligonucleotide (G1 in FIG. 3) can bring two adjacent binding guide conjugates into even closer proximity thus increasing the likelihood that a second guide oligonucleotide (G2 in FIG. 3),when added, will anneal to the same two guide oligonucleotides thus giving rise to a amplification target circle following ligation. These approaches actually provide an advantage over single ligation MPRCA methods in reducing the number of non-productive ligation events.

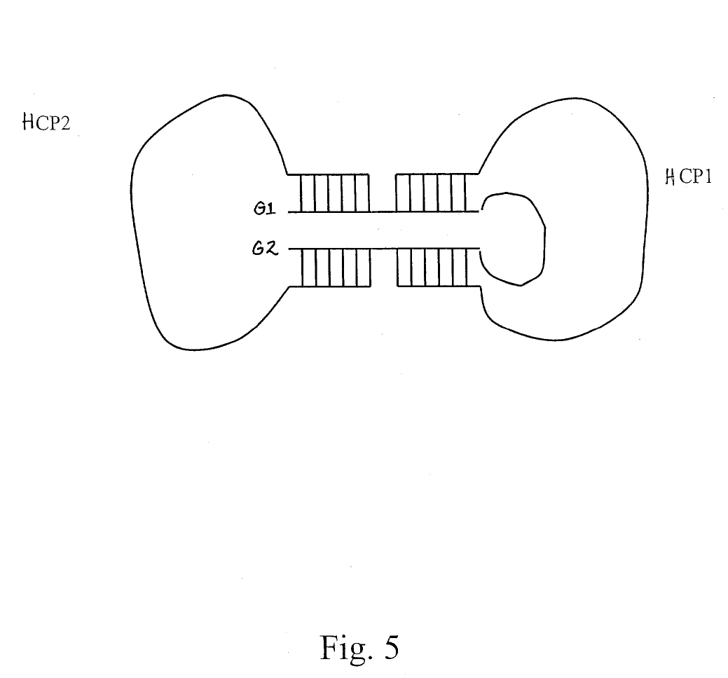

For amplification target circle production it is useful to connect the two guide oligonucleotides by a short loop to facilitate annealing of each half circle probe. FIG. 5

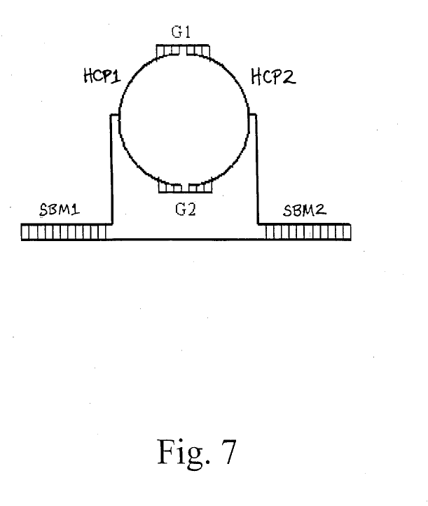

Also, the disclosed method can be used to detect DNA targets by double ligation proximity mediated RCA, as is depicted in FIGS. 6 and 7. Here the specific binding molecule of the binding guide conjugate is an oligonucleotide, which can hybridize to a target sequence. In FIG. 6, two binding guide probes are shown annealed to nearby target sequences, which brings both guide oligonucleotides (G1 and G2) into close proximity to facilitate ATC formation following the addition of half circle probes (HCP1 and HCP2) and ligase. The resulting ATC can be detected by eRCA. Similarly, in FIG. 7, two binding guide probes are shown annealed to nearby target sequences, which brings both tethered half circle probes (HCP1 and HCP2) into close proximity to facilitate ATC formation following the addition of guide oligonucleotides (G1 and G2) and ligase. When a binding guide probe hybridizes to a target sequences in close proximity to another binding guide probe, a pair of half circle probes can be hybridized to a pair of guide oligonucleotides and then ligated together forming an amplification target circle.

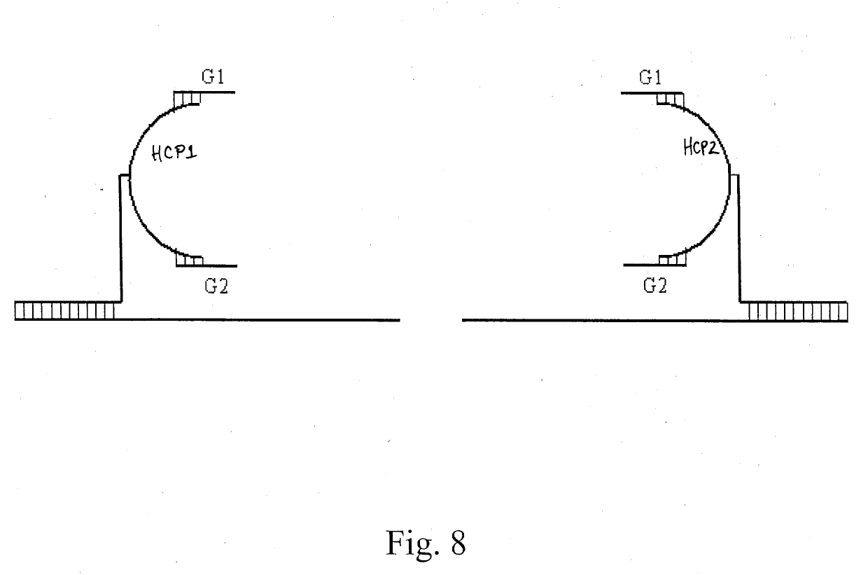

In the presence of excess guide oligonucleotides (G1 and G2 in FIG. 8) unbound and/or non-specifically bound binding guide probes with half circle probes as their oligonucleotide portion can be blocked in a form not suitable for either ligation or further annealing (FIG. 8). Blocking in this manner does not lead to formation of open circle probes by single ligation of half circle probes.

Binding guide conjugates can be used with a solid-state substrate and in combination with combinatorial multicolor coding. For this purpose, samples to be tested can be incorporated into a solid-state sample, as elsewhere herein. The solid-state substrate can be, for example, a glass slide and the solid-state sample preferably incorporates up to 256 individual target or assay samples arranged in dots. Multiple solid-state samples can be used to either test more individual samples, or to increase the number of distinct target sequences to be detected. In the later case, each solid-state sample has an identical set of sample dots, and the assay will be carried out using a different set of binding guide conjugates and half circle probes, collectively referred to as a probe set, for each solid-state sample. This allows a large number of individuals and target sequences to be assayed in a single assay. By using up to six different labels, combinatorial multicolor coding allows up to 63 distinct targets to be detected on a single solid-state sample. When using multiple solid-state substrates and performing RCA with a different set of binding guide conjugates and amplification target circles or half circle probes for each solid-state substrate, the same labels can be used with each solid-state sample (although differences between ATCs or HCPs in each set may require the use of different detection probes). For example, 10 replica slides, each with 256 target sample dots, can be subjected to RCA using 10 different sets of binding guide conjugates and amplification target circles or open circle probes, where each set is designed for combinatorial multicolor coding of 63 targets. This results in an assay for detection of 630 different target molecules.

During or after rolling circle amplification, a cocktail of detection probes can be added, where the cocktail contains color combinations that are specific for each ATC or HCP. The design and combination of such detection probes for use in combinatorial multicolor coding is described elsewhere herein. The labels for combinatorial multicolor detection can be used in the manner of fluorescent change probes. It is preferred that the ATCs or HCPs be designed with combinatorially coded detection tags to allow use of a single set of singly labeled detection probes. It is also preferred that collapsing detection probes be used.

B. Ligation Operation

Double ligation generally entails a set of two separate binding guide conjugates. In some forms, double ligation can entail a set of two separate guide oligonucleotides conjugated to two separate specific binding molecules (such as a specific binding antibody) or a specific binding molecule and an analyte. Alternatively, two separate half circle probes conjugated to two separate specific binding molecules or a specific binding molecule and an analyte. If half circle probes are used in or with the disclosed method, a ligation operation can be used to circularize a pair of half circle probes (and thus form an amplification target circle). Also, a ligation operation can be used to couple one end of a half circle probe to one end of another half circle probe (and thus form an open circle probe). Two half circle probes, optionally in the presence of one or more gap oligonucleotides, can be incubated with a sample containing nucleic acids, under suitable hybridization conditions, and then ligated at one end to form an open circle probe or ligated at both ends to form a covalently closed circle. The ligated and circularized pair of half circle probes is a form of amplification target circle. This operation is similar to ligation of padlock probes described by Nilsson et al., Science, 265:2085-2088 (1994). The ligation operation of two half circle probes to form an ATC allows subsequent amplification to be dependent on the presence and proximity of a guide sequence.

If an open circle probe is used in or with the disclosed method, a ligation operation can be used to circularize the open circle probe (and thus form an amplification target circle). An open circle probe can be prepared from two half circle probes that have been ligated or coupled together at one end. An open circle probe, optionally in the presence of one or more gap oligonucleotides, can be incubated with a sample containing nucleic acids, under suitable hybridization conditions, and then ligated to form a covalently closed circle. The ligated open circle probe is a form of amplification target circle. This operation is similar to ligation of padlock probes described by Nilsson et al., Science, 265:2085-2088 (1994). The ligation operation allows subsequent amplification to be dependent on the presence of a target sequence.

Suitable ligases for the ligation operations of half circle probes or open circle probes are described above. Ligation conditions are generally known. Most ligases require Mg⁺⁺. There are two main types of ligases, those that are ATP-dependent and those that are NAD-dependent. ATP or NAD, depending on the type of ligase, should be present during ligation.

The guide oligonucleotides specific for a half circle probe can be any nucleic acid or other compound to which the guide complement portions of the half circle probe can hybridize in the proper alignment. Guide oligonucleotides can be artificial nucleic acids (or other compounds to which the guide complement portions of the half circle probe can hybridize in the proper alignment). Guide oligonucleotides can also be found in or based on any nucleic acid molecule from any nucleic acid sample. For example, nucleic acid tags can be associated with various of the disclosed compounds to be detected using half circle probes. Thus, a binding guide conjugate can contain a guide oligonucleotide to which a half circle probe can hybridize. In these cases, the guide oligonucleotide provides a link between the target molecule or analyte being detected and the amplification of signal mediated by the half circle probe. When matched half circle probe sets are used, the guide sequences will be related based on the relationship of the half circle probes in the set.

The target sequence for an open circle probe can be any nucleic acid or other compound to which the target probe portions of the open circle probe can hybridize in the proper alignment. Target sequences can be found in any nucleic acid molecule from any nucleic acid sample. Thus, target sequences can be in nucleic acids in cell or tissue samples, reactions, and assays. Target sequences can also be artificial nucleic acids (or other compounds to which the target probe portions of the open circle probe can hybridize in the proper alignment). For example, nucleic acid tags can be associated with various of the disclosed compounds to be detected using open circle probes. Thus, a TS-DNA can contain a target sequence to which an open circle probe can hybridize. In these cases, the target sequence provides a link between the TS-DNA and the amplification of signal mediated by the open circle probe. When matched open circle probe sets are used, the target sequences will be related based on the relationship of the open circle probes in the set.

When RNA is to be detected, it is preferred that a reverse transcription operation be performed to make a DNA target sequence. Alternatively, an RNA target sequence can be detected directly by using a ligase that can perform ligation on a DNA:RNA hybrid substrate. A preferred ligase for this is T4 DNA ligase.

C. Rolling Circle Amplification

Some forms of the disclosed method involve rolling circle amplification. Rolling circle amplification refers to nucleic acid amplification reactions where a circular nucleic acid template is replicated in a single long strand with tandem repeats of the sequence of the circular template. This first, directly produced tandem repeat strand is referred to as tandem sequence DNA (TS-DNA) and its production is referred to as rolling circle replication. Rolling circle amplification refers both to rolling circle replication and to processes involving both rolling circle replication and additional forms of amplification. For example, tandem sequence DNA can be replicated to form complementary strands referred to a secondary tandem sequence DNA. Secondary tandem sequence DNA can, in turn, be replicated, and so on. Tandem sequence DNA can also be transcribed. Rolling circle amplification involving production of only the first tandem sequence DNA (that is, the replicated strand produced by rolling circle replication) can be referred to as linear rolling circle amplification (where “linear” refers to the general amplification kinetics of the amplification).

When rolling circle amplification is involved the rolling circle replication primer and the rolling circle template must be associated together. This typically can occur through mixing one or more amplification target circles with the rolling circle replication primers under conditions that promote association of the rolling circle replication primers with the amplification target circles. To get replication of the amplification target circles the amplification target circle and the rolling circle replication primer typically are incubated under conditions that promote replication of the amplification target circles, wherein replication of the amplification target circles results in the formation of tandem sequence DNA. There are numerous variations of rolling circle amplification that can be used in the disclosed methods. Some useful variations of rolling circle amplification are described in, for example, U.S. Pat. Nos. 5,563,912, 6,143,495, and 6,316,229. In some embodiments the tandem sequence DNA can itself be replicated or otherwise amplified.

In the disclosed method, the amplification or amplification products are detected during the amplification reaction or operation. That is, the progress of amplification or amplification products are detected in real-time. This can be accomplished in any suitable manner, but preferably involves the use of one or more fluorescent change probes and/or one or more fluorescent change primers.

D. Amplification Operation

The basic form of the amplification operation is rolling circle replication of a circular DNA molecule (that is, a circularized pair of half circle probes, a circularized open circle probe, or an amplification target circle). Rolling circle amplification generally requires use of one or more rolling circle replication primers, which are complementary to the primer complement portions of the ATC, and a rolling circle DNA polymerase. The DNA polymerase catalyzes primer extension and strand displacement in a processive rolling circle polymerization reaction that proceeds as long as desired, generating a large DNA molecule that contains a large number of tandem copies of a sequence complementary to the amplification target circle. Some forms of the disclosed method use rolling circle replication primers and secondary DNA strand displacement primers in the amplification reaction.

In multiply-primed RCA, one or more rolling circle replication primers anneal at various places on an amplification target circle to generate multiple replication forks. As each strand grows, the DNA polymerase encounters an adjacent replicating strand and displaces it from the amplification target circle. The result is multiple copies of each circle being produced simultaneously. Multiply-primed RCA can be performed using a single primer (which hybridizes to multiple sites on the amplification target circle) or multiple primers (each of which can hybridize to a single site on the amplification target circle or multiple sites on the amplification target circle). Multiple priming (as occurs in MPRCA) can increase the yield of amplified product from RCA. Primers anneal to multiple locations on the circular template and a product of extension by polymerase is initiated from each location. In this way, multiple extensions are achieved simultaneously from a single amplification target circle.

The amplification operation also involves detection of amplification during the amplification operation (that is, real-time detection). This can be accomplished in any suitable manner. A particularly useful means of obtaining real-time detection is the use of fluorescent change probes and/or primers in the amplification operation. With suitably designed fluorescent change probes and primers, fluorescent signals can be generated as amplification proceeds. In most such cases, the fluorescent signals will be in proportion to the amount of amplification product and/or amount of target sequence or target molecule.

In the disclosed method, detection generally will be during rolling circle amplification and preferably is accomplished through the use of fluorescent changes probes and/or primers. For example, rolling circle replication primers and/or secondary DNA strand displacement primers can be fluorescent change primers. Alternatively or in addition, detection probes that are fluorescent change probes can be used.

As well as rolling circle replication, the amplification operation can include additional nucleic acid replication or amplification processes. For example, TS-DNA can itself be replicated to form secondary TS-DNA. This process is referred to as secondary DNA strand displacement. The combination of rolling circle replication and secondary DNA strand displacement is referred to as linear rolling circle amplification (LRCA). The secondary TS-DNA can itself be replicated to form tertiary TS-DNA in a process referred to as tertiary DNA strand displacement. Secondary and tertiary DNA strand displacement can be performed sequentially or simultaneously. When performed simultaneously, the result is strand displacement cascade amplification. The combination of rolling circle replication and strand displacement cascade amplification is referred to as exponential rolling circle amplification (ERCA). Secondary TS-DNA, tertiary TS-DNA, or both can be amplified by transcription. Exponential rolling circle amplification is a preferred form of amplification operation.

After RCA, a round of ligation-mediated RCA can be performed on the TS-DNA produced in the first RCA. This new round of LM-RCA can be performed with an open circle probe, referred to as a secondary open circle probe, having target probe portions complementary to a target sequence in the TS-DNA produced in the first round. When such new rounds of LM-RCA are performed, the amplification is referred to as nested LM-RCA. Nested LM-RCA can also be performed on ligated OCPs, HCPs, or ATCs that have not been amplified. In this case, LM-RCA can be carried out using either ATCs or target-dependent ligated OCPs. This is especially useful for in situ detection. For in situ detection, a first, unamplified OCP, which is topologically locked to its target sequence, can be subjected to nested LM-RCA. By not amplifying the first OCP, it can remain hybridized to the target sequence while LM-RCA amplifies a secondary OCP topologically locked to the first OCP. Also, a first, unamplified pair of HCPs, which are topologically locked to their guide oligonucleotide, can be subjected to nested LM-RCA. By not amplifying the first pair of HCPs, they can remain hybridized to the guide oligonucleotide while LM-RCA amplifies a secondary OCP topologically locked to the first pair of HCPs. Nested LM-RCA is described in U.S. Pat. No. 6,143,495.

When two half circle probes are used to form the amplification target circle, the amplification target circle can be formed by proximity-mediated ligation. Where HCPs are used, the tandem sequence DNA consists of alternating guide complement sequences and spacer sequences. Note that the spacer sequence of the TS-DNA is the complement of the sequence between the 3′ guide complement portion and the 5′ guide complement potion of each half circle probe in the ligated pair.

When an open circle probe is used to form the amplification target circle, the amplification target circle can be formed by target-mediated ligation. Where OCPs are used, the tandem sequence DNA consists of alternating target sequence and spacer sequence. Note that the spacer sequence of the TS-DNA is the complement of the sequence between the left target probe and the right target probe in the original open circle probe.

1. DNA Strand Displacement

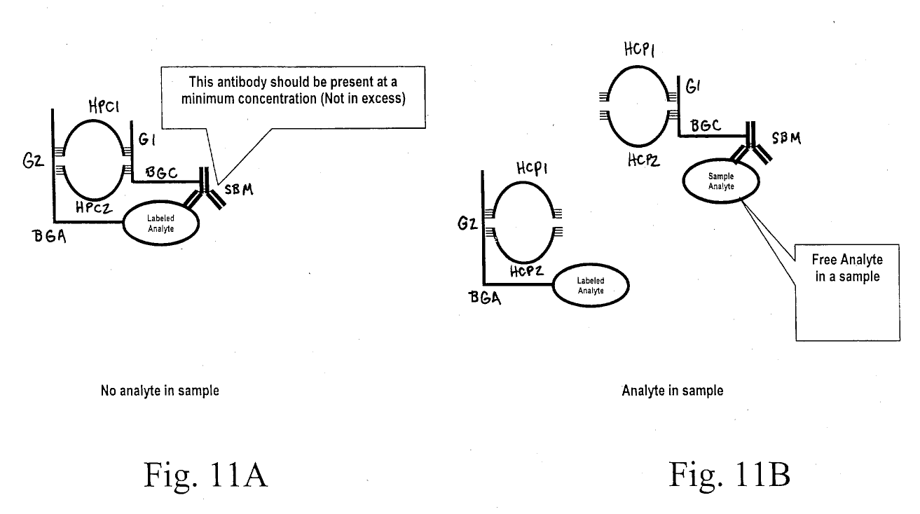

DNA strand displacement is one way to amplify TS-DNA. Secondary DNA strand displacement is accomplished by hybridizing secondary DNA strand displacement primers to TS-DNA and allowing a DNA polymerase to synthesize DNA from these primed sites (see FIG. 11 in U.S. Pat. No. 6,143,495). Because a complement of the secondary DNA strand displacement primer occurs in each repeat of the TS-DNA, secondary DNA strand displacement can result in a high level of amplification. The product of secondary DNA strand displacement is referred to as secondary tandem sequence DNA or TS-DNA-2. Secondary DNA strand displacement can be accomplished by performing RCA to produce TS-DNA, mixing secondary DNA strand displacement primer with the TS-DNA, and incubating under conditions promoting replication of the tandem sequence DNA.

Secondary DNA strand displacement can also be carried out simultaneously with rolling circle replication. This is accomplished by mixing secondary DNA strand displacement primer with the reaction prior to rolling circle replication. As a secondary DNA strand displacement primer is elongated, the DNA polymerase will run into the 5′ end of the next hybridized secondary DNA strand displacement molecule and will displace its 5′ end. In this fashion a tandem queue of elongating DNA polymerases is formed on the TS-DNA template. As long as the rolling circle reaction continues, new secondary DNA strand displacement primers and new DNA polymerases are added to TS-DNA at the growing end of the rolling circle. The generation of TS-DNA-2 and its release into solution by strand displacement is shown diagrammatically in FIG. 11 in U.S. Pat. No. 6,143,495. For simultaneous rolling circle replication and secondary DNA strand displacement, it is preferred that the rolling circle DNA polymerase be used for both replications. This allows optimum conditions to be used and results in displacement of other strands being synthesized downstream. Secondary DNA strand displacement can follow any DNA replication operation, such as RCA, LM-RCA or nested LM-RCA.

Generally, secondary DNA strand displacement can be performed by, simultaneous with or following RCA, mixing a secondary DNA strand displacement primer with the reaction mixture and incubating under conditions that promote both hybridization between the tandem sequence DNA and the secondary DNA strand displacement primer, and replication of the tandem sequence DNA, where replication of the tandem sequence DNA results in the formation of secondary tandem sequence DNA.

When secondary DNA strand displacement is carried out in the presence of a tertiary DNA strand displacement primer (or an equivalent primer), an exponential amplification of TS-DNA sequences takes place. This special and preferred mode of DNA strand displacement is referred to as strand displacement cascade amplification (SDCA) and is a form of exponential rolling circle amplification (ERCA). In SDCA, a secondary DNA strand displacement primer primes replication of TS-DNA to form TS-DNA-2, as described above. The tertiary DNA strand displacement primer strand can then hybridize to, and prime replication of, TS-DNA-2 to form TS-DNA-3. Strand displacement of TS-DNA-3 by the adjacent, growing TS-DNA-3 strands makes TS-DNA-3 available for hybridization with secondary DNA strand displacement primer. This results in another round of replication resulting in TS-DNA-4 (which is equivalent to TS-DNA-2). TS-DNA-4, in turn, becomes a template for DNA replication primed by tertiary DNA strand displacement primer. The cascade continues this manner until the reaction stops or reagents become limiting. This reaction amplifies DNA at an almost exponential rate. In a useful mode of SDCA, the rolling circle replication primers serve as the tertiary DNA strand displacement primer, thus eliminating the need for a separate primer. The additional forms of tandem sequence DNA beyond secondary tandem sequence DNA are collectively referred to herein as higher order tandem sequence DNA. Higher order tandem sequence DNA encompasses TS-DNA-3, TS-DNA-4, and any other tandem sequence DNA produced from replication of secondary tandem sequence DNA or the products of such replication.

For this mode, the rolling circle replication primer should be used at a concentration sufficiently high to obtain rapid priming on the growing TS-DNA-2 strands. To optimize the efficiency of SDCA, it is preferred that a sufficient concentration of secondary DNA strand displacement primer and tertiary DNA strand displacement primer be used to obtain sufficiently rapid priming of the growing TS-DNA strand to out compete TS-DNA for binding to its complementary TS-DNA. Optimization of primer concentrations are described in U.S. Pat. No. 6,143,495 and can be aided by analysis of hybridization kinetics (Young and Anderson, “Quantitative analysis of solution hybridization” in Nucleic Acid Hybridization: A Practical Approach (IRL Press, 1985) pages 47-71).

Generally, strand displacement cascade amplification can be performed by, simultaneous with, or following, RCA, mixing a secondary DNA strand displacement primer and a tertiary DNA strand displacement primer with the reaction mixture and incubating under conditions that promote hybridization between the tandem sequence DNA and the secondary DNA strand displacement primer, replication of the tandem sequence DNA—where replication of the tandem sequence DNA results in the formation of secondary tandem sequence DNA—hybridization between the secondary tandem sequence DNA and the tertiary DNA strand displacement primer, and replication of secondary tandem sequence DNA—where replication of the secondary tandem sequence DNA results in formation of tertiary tandem sequence DNA (TS-DNA-3).

Secondary and tertiary DNA strand displacement can also be carried out sequentially. Following a first round of secondary DNA strand displacement, a tertiary DNA strand displacement primer can be mixed with the secondary tandem sequence DNA and incubated under conditions that promote hybridization between the secondary tandem sequence DNA and the tertiary DNA strand displacement primer, and replication of secondary tandem sequence DNA, where replication of the secondary tandem sequence DNA results in formation of tertiary tandem sequence DNA (TS-DNA-3). This round of strand displacement replication can be referred to as tertiary DNA strand displacement. However, all rounds of strand displacement replication following rolling circle replication can also be referred to collectively as DNA strand displacement or secondary DNA strand displacement.

A modified form of secondary DNA strand displacement results in amplification of TS-DNA and is referred to as opposite strand amplification (OSA). OSA is the same as secondary DNA strand displacement except that a special form of rolling circle replication primer is used that prevents it from hybridizing to TS-DNA-2. Opposite strand amplification is described in U.S. Pat. No. 6,143,495.

The DNA generated by DNA strand displacement can be labeled and/or detected using the same labels, labeling methods, and detection methods described for use with TS-DNA. In the disclosed method, detection generally will be during DNA strand displacement and preferably is accomplished through the use of fluorescent changes probes and/or primers. For example, secondary DNA strand displacement primers and/or tertiary DNA strand displacement primers can be fluorescent change primers. Alternatively or in addition, detection probes that are fluorescent change probes can be used.

2. Geometric Rolling Circle Amplification

RCA reactions can be carried out with either linear or geometric kinetics (Lizardi et al., 1998). Linear rolling circle amplification generally follows linear kinetics. Two useful forms of RCA with geometric kinetics are exponential multiply-primed rolling circle amplification (EMPRCA) and exponential rolling circle amplification (ERCA). In exponential multiply-primed RCA, one or more rolling circle replication primers anneal at various places on the amplification target circle to generate multiple replication forks. As each strand grows, the DNA polymerase encounters an adjacent replicating strand and displaces it from the amplification target circle. The result is multiple copies of each circle being produced simultaneously. The replicated strands are referred to as tandem sequence DNA (TS-DNA). As each TS-DNA strand is displaced from the circular template, secondary DNA strand displacement primers can anneal to, and prime replication of, the TS-DNA. Replication of the TS-DNA forms complementary strands referred to as secondary tandem sequence DNA or TS-DNA-2. As a secondary TS-DNA strand is elongated, the DNA polymerase will run into the 5′ end of the next growing strand of secondary TS-DNA and will displace its 5′ end. In this fashion a tandem queue of elongating DNA polymerases is formed on the TS-DNA template. As long as the rolling circle reaction continues, new primers and new DNA polymerases are added to TS-DNA at the growing end of the rolling circle.

Random or degenerate primers can be used to perform multiply-primed RCA. Such random or degenerate primers will anneal to multiple sites on the amplification target circle (resulting in production of tandem sequence DNA), as well as to multiple sites on the tandem sequence DNA (resulting in production of secondary tandem sequence DNA). The random primers can then hybridize to, and prime replication of, TS-DNA-2 to form TS-DNA-3 (which is equivalent to the original TS-DNA). Strand displacement of TS-DNA-3 by the adjacent, growing TS-DNA-3 strands makes TS-DNA-3 available for hybridization with the primers. This can result in another round of replication resulting in TS-DNA-4 (which is equivalent to TS-DNA-2). TS-DNA-4, in turn, becomes a template for DNA replication primed by random primers. The cascade continues in this manner until the reaction stops or reagents become limiting. Multiply-primed RCA is particularly useful for amplifying larger circular templates such as amplification target circles that are, or are derived from or include, nucleic acid molecules of interest. Multiply-primed RCA is described in Dean et al., Rapid Amplification of Plasmid and Phage DNA Using Phi29 DNA Polymerase and Multiply-Primed Rolling Circle Amplification, Genome Research 11:1095-1099 (2001).

Exponential multiply-primed RCA also can be achieved using specific rolling circle replication primers, secondary DNA strand displacement primers and tertiary DNA strand displacement primers. In this form of the disclosed method, rolling circle replication is primed from multiple specific primer complement portions of the circular template. As the strand grows, the DNA polymerase encounters 5′ end of the strand and displaces it from the circular template. A secondary DNA strand displacement primer primes replication of TS-DNA to form a complementary strand referred to as secondary tandem sequence DNA or TS-DNA-2. As a secondary DNA strand displacement primer is elongated, the DNA polymerase will run into the 5′ end of the next hybridized secondary DNA strand displacement molecule and will displace its 5′ end. In this fashion a tandem queue of elongating DNA polymerases is formed on the TS-DNA template. As long as the rolling circle reaction continues, new secondary DNA strand displacement primers and new DNA polymerases are added to TS-DNA at the growing end of the rolling circle. A tertiary DNA strand displacement primer strand (which is complementary to the TS-DNA-2 strand and which can be the rolling circle replication primer) can then hybridize to, and prime replication of, TS-DNA-2 to form TS-DNA-3 (which is equivalent to the original TS-DNA). Strand displacement of TS-DNA-3 by the adjacent, growing TS-DNA-3 strands makes TS-DNA-3 available for hybridization with secondary DNA strand displacement primer. This results in another round of replication resulting in TS-DNA-4 (which is equivalent to TS-DNA-2). TS-DNA-4, in turn, becomes a template for DNA replication primed by tertiary DNA strand displacement primer. The cascade continues in this manner until the reaction stops or reagents become limiting. In one mode of ERCA, the rolling circle replication primer serves as the tertiary DNA strand displacement primer, thus eliminating the need for a separate primer. Exponential RCA and other useful forms of RCA are described in U.S. Pat. Nos. 5,854,033, and 6,143,495.

E. Detection of Amplification Products

Products of the amplification operation can be detected using any nucleic acid detection technique. For real-time detection, the amplification products and the progress of amplification are detected during the amplification operation. Real-time detection is usefully accomplished using one or more or one or a combination of fluorescent change probes and fluorescent change primers. Other detection techniques can be used, either alone or in combination with real-timer detection and/or detection involving fluorescent change probes and primers. Many techniques are known for detecting nucleic acids. The nucleotide sequence of the amplified sequences also can be determined using any suitable technique.

1. Primary Labeling

Primary labeling consists of incorporating labeled moieties, such as fluorescent nucleotides, biotinylated nucleotides, digoxygenin-containing nucleotides, or bromodeoxyuridine, during rolling circle replication in RCA, or during transcription in RCT. For example, fluorescent labels can be incorporated into replicated nucleic acid by using fluorescently labeled primers, such as fluorescent change rolling circle replication primers. In another example, one can incorporate cyanine dye UTP analogs (Yu et al. (1994)) at a frequency of 4 analogs for every 100 nucleotides. A preferred method for detecting nucleic acid amplified in situ is to label the DNA during amplification with BrdUrd, followed by binding of the incorporated BUDR with a biotinylated anti-BUDR antibody (Zymed Labs, San Francisco, Calif.), followed by binding of the biotin moieties with Streptavidin-Peroxidase (Life Sciences, Inc.), and finally development of fluorescence with Fluorescein-tyramide (DuPont de Nemours & Co., Medical Products Dept.).

A useful form of primary labeling is the use of fluorescent change primers in the amplification operation. Fluorescent change primers exhibit a change in fluorescence intensity or wavelength based on a change in the form or conformation of the primer and the amplified nucleic acid. Stem quenched primers are primers that when not hybridized to a complementary sequence form a stem structure (either an intramolecular stem structure or an intermolecular stem structure) that brings a fluorescent label and a quenching moiety into proximity such that fluorescence from the label is quenched. When the primer binds to a complementary sequence, the stem is disrupted, the quenching moiety is no longer in proximity to the fluorescent label and fluorescence increases. In the disclosed method, stem quenched primers are used as primers for nucleic acid synthesis and thus become incorporated into the synthesized or amplified nucleic acid. Examples of stem quenched primers are peptide nucleic acid quenched primers and hairpin quenched primers.

Peptide nucleic acid quenched primers are primers associated with a peptide nucleic acid quencher or a peptide nucleic acid fluor to form a stem structure. The primer contains a fluorescent label or a quenching moiety and is associated with either a peptide nucleic acid quencher or a peptide nucleic acid fluor, respectively. This puts the fluorescent label in proximity to the quenching moiety. When the primer is replicated, the peptide nucleic acid is displaced, thus allowing the fluorescent label to produce a fluorescent signal.

Hairpin quenched primers are primers that when not hybridized to a complementary sequence form a hairpin structure (and, typically, a loop) that brings a fluorescent label and a quenching moiety into proximity such that fluorescence from the label is quenched. When the primer binds to a complementary sequence, the stem is disrupted, the quenching moiety is no longer in proximity to the fluorescent label and fluorescence increases. Hairpin quenched primers are typically used as primers for nucleic acid synthesis and thus become incorporated into the synthesized or amplified nucleic acid. Examples of hairpin quenched primers are Amplifluor primers and scorpion primers.

Cleavage activated primers are primers where fluorescence is increased by cleavage of the primer. Generally, cleavage activated primers are incorporated into replicated strands and are then subsequently cleaved. Cleavage activated primers can include a fluorescent label and a quenching moiety in proximity such that fluorescence from the label is quenched. When the primer is clipped or digested (typically by the 5′-3′ exonuclease activity of a polymerase during amplification), the quenching moiety is no longer in proximity to the fluorescent label and fluorescence increases. Little et al., Clin. Chem. 45:777-784 (1999), describe the use of cleavage activated primers.

2. Secondary Labeling

Secondary labeling consists of using suitable molecular probes, such as detection probes, to detect the amplified nucleic acids. For example, an amplification target circle may be designed to contain several repeats of a known arbitrary sequence, referred to as detection tags. The detection probes can then be hybridized to these detection tags. The detection probes may be labeled as described above with, for example, an enzyme, fluorescent moieties, or radioactive isotopes. By using three detection tags per amplification target circle, and four fluorescent moieties per each detection probe, one may obtain a total of twelve fluorescent signals for every amplification target circle repeat in the TS-DNA, yielding a total of 12,000 fluorescent moieties for every amplification target circle that is amplified by RCA. Detection probes can interact by hybridization or annealing via normal Watson-Crick base-pairing (or related alternatives) or can interact with double-stranded targets to form a triple helix. Such triplex-forming detection probes can be used in the same manner as other detection probes, such as in the form of fluorescent change probes.

A useful form of secondary labeling is the use of fluorescent change probes and primers in or following the amplification operation. Hairpin quenched probes are probes that when not bound to a target sequence form a hairpin structure (and, typically, a loop) that brings a fluorescent label and a quenching moiety into proximity such that fluorescence from the label is quenched. When the probe binds to a target sequence, the stem is disrupted, the quenching moiety is no longer in proximity to the fluorescent label and fluorescence increases. Examples of hairpin quenched probes are molecular beacons, fluorescent triplex oligos, triplex molecular beacons, triplex FRET probes, and QPNA probes.

Cleavage activated probes are probes where fluorescence is increased by cleavage of the probe. Cleavage activated probes can include a fluorescent label and a quenching moiety in proximity such that fluorescence from the label is quenched. When the probe is clipped or digested (typically by the 5′-3′ exonuclease activity of a polymerase during or following amplification), the quenching moiety is no longer in proximity to the fluorescent label and fluorescence increases. TaqMan probes are an example of cleavage activated probes.

Cleavage quenched probes are probes where fluorescence is decreased or altered by cleavage of the probe. Cleavage quenched probes can include an acceptor fluorescent label and a donor moiety such that, when the acceptor and donor are in proximity, fluorescence resonance energy transfer from the donor to the acceptor causes the acceptor to fluoresce. The probes are thus fluorescent, for example, when hybridized to a target sequence. When the probe is clipped or digested (typically by the 5′-3′ exonuclease activity of a polymerase during or after amplification), the donor moiety is no longer in proximity to the acceptor fluorescent label and fluorescence from the acceptor decreases. If the donor moiety is itself a fluorescent label, it can release energy as fluorescence (typically at a different wavelength than the fluorescence of the acceptor) when not in proximity to an acceptor. The overall effect would then be a reduction of acceptor fluorescence and an increase in donor fluorescence. Donor fluorescence in the case of cleavage quenched probes is equivalent to fluorescence generated by cleavage activated probes with the acceptor being the quenching moiety and the donor being the fluorescent label. Cleavable FRET (fluorescence resonance energy transfer) probes are an example of cleavage quenched probes.

Fluorescent activated probes are probes or pairs of probes where fluorescence is increased or altered by hybridization of the probe to a target sequence. Fluorescent activated probes can include an acceptor fluorescent label and a donor moiety such that, when the acceptor and donor are in proximity (when the probes are hybridized to a target sequence), fluorescence resonance energy transfer from the donor to the acceptor causes the acceptor to fluoresce. Fluorescent activated probes are typically pairs of probes designed to hybridize to adjacent sequences such that the acceptor and donor are brought into proximity. Fluorescent activated probes can also be single probes containing both a donor and acceptor where, when the probe is not hybridized to a target sequence, the donor and acceptor are not in proximity but where the donor and acceptor are brought into proximity when the probe hybridized to a target sequence. This can be accomplished, for example, by placing the donor and acceptor on opposite ends a the probe and placing target complement sequences at each end of the probe where the target complement sequences are complementary to adjacent sequences in a target sequence. If the donor moiety of a fluorescent activated probe is itself a fluorescent label, it can release energy as fluorescence (typically at a different wavelength than the fluorescence of the acceptor) when not in proximity to an acceptor (that is, when the probes are not hybridized to the target sequence). When the probes hybridize to a target sequence, the overall effect would then be a reduction of donor fluorescence and an increase in acceptor fluorescence. FRET probes are an example of fluorescent activated probes. Stem quenched primers (such as peptide nucleic acid quenched primers and hairpin quenched primers) can be used as secondary labels.

3. Multiplexing and Hybridization Array Detection

RCA is easily multiplexed by using sets of different amplification target circles, each amplification target circle being associated with, for example, different target molecules, target sequences, and/or array positions. Each amplification target circle can have a different primer complement portions and/or different detection tag portions corresponding to different rolling circle replication primers and/or different detection probes. Use of different fluorescent labels with different rolling circle replication primers and/or different detection probes allows specific detection of different open circle probes (and thus, of different targets).

For multiplexing, the mixture of amplification target circle(s), rolling circle replication primer(s) and fluorescent change probe(s) in the disclosed method can comprise a plurality of amplification target circles. The fluorescent change probes each can comprise a complementary portion, the amplification target circles each can comprise at least one detection tag portion, and the complementary portion of each of the fluorescent change probes matches the sequence of one or more of the detection tag portions of the amplification target circles. The mixture can comprise a plurality of fluorescent change probes, where the complementary portion of each fluorescent change probe matches the sequence of one or more of the detection tag portions of a different one of the amplification target circles. The mixture can comprise a plurality of fluorescent change probes, where the complementary portion of each fluorescent change probe matches the sequence of one or more of the detection tag portions of one or more of the amplification target circles. The mixture can comprise a plurality of fluorescent change probes, where the complementary portion of each fluorescent change probe matches the sequence of one of the detection tag portions of a different one of the amplification target circles. The mixture can comprise a plurality of fluorescent change probes, where the complementary portion of each fluorescent change probe matches the sequence of a plurality of the detection tag portions of a different one of the amplification target circles. The mixture can comprise a plurality of fluorescent change probes, where the complementary portion of each fluorescent change probe matches the sequence of a plurality of the detection tag portions of one of the amplification target circles. The mixture can comprise a plurality of fluorescent change probes, where the complementary portion of each fluorescent change probe matches the sequence of a plurality of the detection tag portions of a plurality of the amplification target circles. The mixture can comprise a plurality of fluorescent change probes, where the complementary portion of each fluorescent change probe matches the sequence of one of the detection tag portions of a plurality of the amplification target circles. The mixture can comprise a plurality of fluorescent change probes, where the complementary portion of each fluorescent change probe matches the sequence of one of the detection tag portions of one of the amplification target circles.

RCA can be multiplexed by, for example, using sets of different half circle probes, each half circle probe carrying different guide complement sequences designed for binding to unique targets and each half circle probe having a different primer complement portions and/or different detection tag portions corresponding to different rolling circle replication primers and/or different detection probes. Only those half circle probes that are able to find their targets will give rise to TS-DNA. Use of different fluorescent labels with different rolling circle replication primers and/or different detection probes allows specific detection of different half circle probes (and thus, of different targets).

RCA can also be multiplexed by using sets of different open circle probes, each open circle probe carrying different target probe sequences designed for binding to unique targets and each open circle probe having a different primer complement portions and/or different detection tag portions corresponding to different rolling circle replication primers and/or different detection probes. Only those open circle probes that are able to find their targets will give rise to TS-DNA. Use of different fluorescent labels with different rolling circle replication primers and/or different detection probes allows specific detection of different open circle probes (and thus, of different targets).

The TS-DNA molecules generated by RCA are of high molecular weight and low complexity; the complexity being the length of the amplification target circle. There are several ways to capture a given TS-DNA to a fixed position in a solid-state detector. One is to include within the amplification target circles a unique address tag sequence for each unique amplification target circle. TS-DNA generated from a given amplification target circle will then contain sequences corresponding to a specific address tag sequence. Another way to capture TS-DNA when open circle probes are used is to use the target sequence present on the TS-DNA as the address tag. Also, another way to capture TS-DNA when half circle probes are used is to use the target sequence present on the TS-DNA as the address probe.

4. Detecting Multiple Amplification Target Circles

Multiplex RCA assays are useful for detecting multiple amplification target circles. A single RCA assay can be used to detect the presence of one or more members of a group of any number of amplification target circles (and, thus, any number of corresponding target sequences, target molecules, or analytes). By associating different amplification target circles with different target molecules (using binding guide conjugates specific for the target molecules), each different target molecule can be detected by differential detection of the various amplification target circles. This can be accomplished, for example, by designing an amplification target circle for each target molecule, where the detection tag portions and/or the primer complement portions of each amplification target circle are different. Amplification of the different ATCs can be detected based on different primer complement portion sequences by using, for example, rolling circle replication primers that are fluorescent change primers. Alternatively, the different amplification target circles can be detected based on different detection tag sequences by using, for example, detection probes that are fluorescent change probes. In this case, the primer portions of all the amplification target circles can be the same. Use of different detection tag sequences and different detection probes also allows differential detection of amplification target circles even when random or degenerate primers are used for multiply-primed RCA. Different detection probes can be used to detect the various TS-DNAs (each having specific detection tag sequences).

By associating different guide oligonucleotides, different half circle probes, or different amplification target circles with different target molecules, such as proteins (using binding guide conjugates specific for the proteins of interest), each different target molecule can be detected by differential detection of the various guide oligonucleotides or half circle probes. This can be accomplished, for example, by designing half circle probes (and associated gap oligonucleotides, if desired) for each guide oligonucleotide in the group, where the guide complement portions and the detection primer complement portions present in each pair of half circle probes are different but the sequence of the common primer complement portions and secondary DNA strand displacement matching portions that are present in all of the ligated pairs of half circle probes are the same. All of the half circle probes are placed in the same HCP-target sample mixture, and the same primers are used to amplify. For each pair of guide sequences present in the assay (for example, those associated with proteins present in the target sample and where that association brings one guide sequence in the pair in close proximity to the other), the two HCPs for that pair of guide sequences will be ligated into a circle and the circle will be amplified to form TS-DNA. Since the detection primer complement portions are different, amplification of the different HCPs can be detected (using, for example, rolling circle replication primers that are fluorescent change primers). Alternatively, the half circle probes can each target a different guide sequence in the group, where the guide complement portions and the sequence of the detection tag portions present in each pair of half circle probes when they are ligated together are different but the sequence of the primer complement portions that are present in all of the ligated pairs of half circle probes are the same. Different detection probes are used to detect the various TS-DNAs (each having specific detection tag sequences). For each pair of guide sequences present in the assay (for example, those associated with proteins present in the target sample and where that association brings one guide sequence in the pair in close proximity to the other), the two HCPs for that pair of guide sequences will be ligated into a circle and the circle will be amplified to form TS-DNA. Since the detection tags on TS-DNA resulting from amplification of the HCPs are different, TS-DNA resulting from ligation of each pair of HCPs can be detected individually in that assay.

5. Combinatorial Multicolor Coding

One form of multiplex detection involves the use of a combination of labels that either fluoresce at different wavelengths or are colored differently. One of the advantages of fluorescence for the detection of hybridization probes is that several targets can be visualized simultaneously in the same sample. Using a combinatorial strategy, many more targets can be discriminated than the number of spectrally resolvable fluorophores. Combinatorial labeling provides the simplest way to label probes in a multiplex fashion since a probe fluor is either completely absent (−) or present in unit amounts (+); image analysis is thus more amenable to automation, and a number of experimental artifacts, such as differential photobleaching of the fluors and the effects of changing excitation source power spectrum, are avoided. Combinatorial labeling can be used with fluorescent change probes and primers.

The combinations of labels establish a code for identifying different detection probes and, by extension, different target molecules to which those detection probes are associated with. This labeling scheme is referred to as Combinatorial Multicolor Coding (CMC). Such coding is described by Speicher et al., Nature Genetics 12:368-375 (1996). Use of CMC in connection with rolling circle amplification is described in U.S. Pat. No. 6,143,495. Any number of labels, which when combined can be separately detected, can be used for combinatorial multicolor coding. It is preferred that 2, 3, 4, 5, or 6 labels be used in combination. It is most preferred that 6 labels be used. The number of labels used establishes the number of unique label combinations that can be formed according to the formula 2^{N}−1, where N is the number of labels. According to this formula, 2 labels forms three label combinations, 3 labels forms seven label combinations, 4 labels forms 15 label combinations, 5 labels form 31 label combinations, and 6 labels forms 63 label combinations.

For combinatorial multicolor coding, a group of different detection probes are used as a set. Each type of detection probe in the set is labeled with a specific and unique combination of fluorescent labels. For those detection probes assigned multiple labels, the labeling can be accomplished by labeling each detection probe molecule with all of the required labels. Alternatively, pools of detection probes of a given type can each be labeled with one of the required labels. By combining the pools, the detection probes will, as a group, contain the combination of labels required for that type of detection probe. Where each detection probe is labeled with a single label, label combinations can also be generated by using HCPs, OCPs, or ATCs with coded combinations of detection tags complementary to the different detection probes. In this scheme, the HCPs, OCPs, or ATCs will contain a combination of detection tags representing the combination of labels required for a specific label code. Further illustrations are described in U.S. Pat. No. 6,143,495. Use of pools of detection probes each probe with a single label is preferred when fluorescent change probes are used.

Speicher et al. describes a set of fluors and corresponding optical filters spaced across the spectral interval 350-770 nm that give a high degree of discrimination between all possible fluor pairs. This fluor set, which is preferred for combinatorial multicolor coding, consists of 4′-6-diamidino-2-phenylinodole (DAPI), fluorescein (FITC), and the cyanine dyes Cy3, Cy3.5, Cy5, Cy5.5 and Cy7. Any subset of this preferred set can also be used where fewer combinations are required. The absorption and emission maxima, respectively, for these fluors are: DAPI (350 nm; 456 nm), FITC (490 nm; 520 nm), Cy3 (554 nm; 568 nm), Cy3.5 (581 nm; 588 nm), Cy5 (652 nm; 672 nm), Cy5.5 (682 nm; 703 nm) and Cy7 (755 nm; 778 nm). The excitation and emission spectra, extinction coefficients and quantum yield of these fluors are described by Ernst et al., Cytometry 10:3-10 (1989), Mujumdar et al., Cytometry 10:11-19 (1989), Yu, Nucleic Acids Res. 22:3226-3232 (1994), and Waggoner, Meth. Enzymology 246:362-373 (1995). These fluors can all be excited with a 75W Xenon arc.

To attain selectivity, filters with bandwidths in the range of 5 to 16 nm are preferred. To increase signal discrimination, the fluors can be both excited and detected at wavelengths far from their spectral maxima. Emission bandwidths can be made as wide as possible. For low-noise detectors, such as cooled CCD cameras, restricting the excitation bandwidth has little effect on attainable signal to noise ratios. A list of preferred filters for use with the preferred fluor set is listed in Table 1 of Speicher et al. It is important to prevent infra-red light emitted by the arc lamp from reaching the detector; CCD chips are extremely sensitive in this region. For this purpose, appropriate IR blocking filters can be inserted in the image path immediately in front of the CCD window to minimize loss of image quality. Image analysis software can then be used to count and analyze the spectral signatures of fluorescent dots.

F. Gap-Filling Ligation

The gap space formed by two HCPs hybridized to a guide sequence or an OCP hybridized to a target sequence is normally occupied by one or more gap oligonucleotides as described above. Such a gap space may also be filled in by a gap-filling DNA polymerase during the ligation operation. As an alternative, the gap space can be partially bridged by one or more gap oligonucleotides, with the remainder of the gap filled using DNA polymerase. This modified ligation operation is referred to herein as gap-filling ligation and is a preferred form of the ligation operation. The principles and procedure for gap-filling ligation are generally analogous to the filling and ligation performed in gap LCR (Wiedmann et al., PCR Methods and Applications (Cold Spring Harbor Laboratory Press, Cold Spring Harbor Laboratory, NY, 1994) pages S51-S64; Abravaya et al., Nucleic Acids Res., 23(4):675-682 (1995); European Patent Application EP0439182 (1991)). In the case of LM-RCA, the gap-filling ligation operation is substituted for the normal ligation operation. Gap-filling ligation provides a means for discriminating between closely related target sequences. Gap-filling ligation can be accomplished by using a different DNA polymerase, referred to herein as a gap-filling DNA polymerase. Suitable gap-filling DNA polymerases are described above. Alternatively, DNA polymerases in general can be used to fill the gap when a stop base is used. The use of stop bases in the gap-filling operation of LCR is described in European Patent Application EP0439182. The principles of the design of gaps and the ends of flanking probes to be joined, as described in EP0439182, is generally applicable to the design of the gap spaces and the ends of target probe portions described herein. Gap-filling ligation is further described in U.S. Pat. No. 6,143,495.

G. Discrimination between Closely Related Target Sequences

Half circle probes, open circle probes, gap oligonucleotides, and gap spaces can be designed to discriminate closely related target sequences, such as genetic alleles, or guide sequences. Where closely related target sequences differ at a single nucleotide, it is preferred that half circle probes or open circle probes (depending on the case) be designed with the complement of this nucleotide occurring at one end of the probe, or at one of the ends of the gap oligonucleotide(s). Where gap-filling ligation is used, it is preferred that the distinguishing nucleotide appear opposite the gap space. This allows incorporation of alternative (that is, allelic) sequence into the ligated HCP or OCP without the need for alternative gap oligonucleotides. Where gap-filling ligation is used with a gap oligonucleotide(s) that partially fills the gap, it is preferred that the distinguishing nucleotide appear opposite the portion of gap space not filled by a gap oligonucleotide. Ligation of gap oligonucleotides with a mismatch at either terminus is extremely unlikely because of the combined effects of hybrid instability and enzyme discrimination. When the TS-DNA is generated, it will carry a copy of the gap oligonucleotide sequence that led to a correct ligation. Gap oligonucleotides may give even greater discrimination between related target sequences in certain circumstances, such as those involving wobble base pairing of alleles. Features of half circle probes or open circle probes and gap oligonucleotides that increase the target-dependency of the ligation operation are generally analogous to such features developed for use with the ligation chain reaction. These features can be incorporated into half circle probes or open circle probes and gap oligonucleotides for use in LM-RCA. In particular, European Patent Application EP0439182 describes several features for enhancing target-dependency in LCR that can be adapted for use in LM-RCA. The use of stop bases in the gap space, as described in European Patent Application EP0439182, is a preferred mode of enhancing the target discrimination of a gap-filling ligation operation.

A preferred form of target sequence discrimination can be accomplished by employing two groups of half circle probe pairs or two types of open circle probes. In one embodiment, a single gap oligonucleotide is used which is the same for both target sequences, that is, the gap oligonucleotide is complementary to both target sequences. In a preferred embodiment, a gap-filling ligation operation can be used (Example 3 in U.S. Pat. No. 6,143,495). Target sequence discrimination would occur by virtue of mutually exclusive ligation events, or extension-ligation events, for which only one of the two groups of half circle probes or one of the two types of open-circle probes is competent. Preferably, the discriminator nucleotide would be located at the penultimate nucleotide from the 3′ end of either of the half circle probes in either pair or at the penultimate nucleotide from the 3′ end of each of the open circle probes. The two pairs of half circle probes or two open circle probes would also contain two different detection tags designed to bind alternative detection probes and/or address probes. Each of the two detection probes would have a different detection label. Both half circle pairs or open circle probes would have the same primer complement portion. Thus, both ligated pairs of half circle probes or ligated open circle probes can be amplified using a single primer. Upon array hybridization, each detection probe would produce a unique signal, for example, two alternative fluorescence colors, corresponding to the alternative target sequences.

These techniques for target sequence discrimination are especially useful within matched half circle probe sets or matched open circle probe sets.

H. Transcription

Once TS-DNA is generated using RCA, further amplification can be accomplished by transcribing the TS-DNA from promoters embedded in the TS-DNA. This combined process, referred to as rolling circle replication with transcription (RCT) requires that the amplification target circle from which the TS-DNA is made have a promoter portion in its spacer region. The promoter portion is then amplified along with the rest of the amplification target circle resulting in a promoter embedded in each tandem repeat of the TS-DNA. Because transcription, like rolling circle amplification, is a process that can go on continuously (with re-initiation), multiple transcripts can be produced from each of the multiple promoters present in the TS-DNA. RCT effectively adds another level of amplification of amplification target circles. RCT is further described in U.S. Pat. No. 6,143,495. Amplification target circles can also be directly transcribed (that is, not in conjunction with rolling circle amplification). The amplified product will be RNA. Transcription of amplification target circles can produce a long tandem repeat transcript if the amplification target circle does not have a transcription termination sequence.

The transcripts generated in RCT or direct transcription can be labeled and/or detected using the same labels, labeling methods, and detection methods described for use with TS-DNA. Most of these labels and methods are adaptable for use with nucleic acids in general. A useful method of labeling RCT transcripts is by direct labeling of the transcripts by incorporation of labeled nucleotides, most preferably biotinylated nucleotides, during transcription. RCT transcripts can also be detected in real-time, using, for example, fluorescent change probes.

I. Specific Embodiments

In some forms, the disclosed method involves (a) bringing into contact one or more analyte samples and one or more binding guide conjugates, wherein each binding guide conjugate comprises a specific binding molecule and a guide oligonucleotide, wherein each specific binding molecule interacts directly or indirectly with an analyte in the analyte sample, and incubating the analyte samples and the binding guide conjugates under conditions that promote interaction of the specific binding molecules and the analytes; (b), prior to, simultaneous with, or following step (a), bringing into contact the binding guide conjugates and one or more half circle probes, wherein each half circle probe comprises a single-stranded DNA molecule comprising two guide complement portions, wherein each guide complement portion is complementary to one of the guide oligonucleotides, and incubating the binding guide conjugates and the half circle probes under conditions that promote hybridization between the guide oligonucleotides and the half circle probes; and (c), following step (b) and prior to, simultaneous with, or following step (a), incubating the binding guide conjugates and half circle probes under conditions that promote ligation of half circle probes, thereby producing one or more amplification target circles.

In some forms, the disclosed method involves (a) bringing into contact one or more analyte samples and one or more arrays, wherein each array comprises a set of analyte capture agents, a set of accessory molecules, or both, wherein each analyte capture agent interacts with an analyte directly or indirectly; (b), prior to, simultaneous with, or following step (a), bringing into contact one or more of the arrays and one or more binding guide conjugates, wherein each binding guide conjugate comprises a specific binding molecule and a guide oligonucleotide, wherein each specific binding molecule interacts directly or indirectly with an analyte in the analyte sample, and incubating the analyte samples and the binding guide conjugates under conditions that promote interaction of the specific binding molecules and the analytes; (c), prior to, simultaneous with, or following steps (a) or (b), bringing into contact the binding guide conjugates and one or more half circle probes, wherein each half circle probe comprises a single-stranded DNA molecule comprising two guide complement portions, wherein each guide complement portion is complementary to at least one of the guide oligonucleotides, and incubating the binding guide conjugates and the half circle probes under conditions that promote hybridization between the guide oligonucleotides and the half circle probes; (d), following step (c) and prior to, simultaneous with, or following steps (a) or (b), incubating the binding guide conjugates and half circle probes under conditions that promote ligation of half circle probes to each other, thereby producing one or more amplification target circles; and (e), following step (d), incubating the amplification target circles under conditions that promote replication of the amplification target circles, wherein replication of the amplification target circles results in the formation of tandem sequence DNA.

In some forms, the disclosed method involves (a) treating one or more analyte samples so that one or more analytes are modified; (b), prior to, simultaneous with, or following step (a), bringing into contact one or more of the analyte samples and one or more binding guide conjugates, wherein each binding guide conjugate comprises a specific binding molecule and a guide oligonucleotide, wherein each specific binding molecule interacts directly or indirectly with an analyte in the analyte sample, and incubating the analyte samples and the binding guide conjugates under conditions that promote interaction of the specific binding molecules and the analytes; (c), prior to, simultaneous with, or following steps (a) or (b), bringing into contact the binding guide conjugates and one or more half circle probes, wherein each half circle probe comprises a single-stranded DNA molecule comprising two guide complement portions, wherein each guide complement portion is complementary to at least one of the guide oligonucleotides, and incubating the binding guide conjugates and the half circle probes under conditions that promote hybridization between the guide oligonucleotides and the half circle probes; (d), following step (c) and prior to, simultaneous with, or following steps (a) or (b), incubating the binding guide conjugates and half circle probes under conditions that promote ligation of half circle probes to each other, thereby producing one or more amplification target circles; and (e), following step (d), incubating the amplification target circles under conditions that promote replication of the amplification target circles, wherein replication of the amplification target circles results in the formation of tandem sequence DNA.

In some forms, the disclosed method involves (a) bringing into contact one or more analyte samples and one or more arrays, wherein each array comprises a set of analyte capture agents, a set of accessory molecules, or both, wherein each analyte capture agent interacts with an analyte directly or indirectly; (b), prior to, simultaneous with, or following step (a), bringing into contact one or more of the analyte samples and one or more binding guide conjugates, wherein each binding guide conjugate comprises a specific binding molecule and a guide oligonucleotide, wherein each specific binding molecule interacts directly or indirectly with an analyte in the analyte sample, and incubating the analyte samples and the binding guide conjugates under conditions that promote interaction of the specific binding molecules and the analytes; (c), prior to, simultaneous with, or following steps (a) or (b), bringing into contact the binding guide conjugates and one or more half circle probes, wherein each half circle probe comprises a single-stranded DNA molecule comprising two guide complement portions, wherein each guide complement portion is complementary to at least one of the guide oligonucleotides, and incubating the binding guide conjugates and the half circle probes under conditions that promote hybridization between the guide oligonucleotides and the half circle probes; (d), following step (c) and prior to, simultaneous with, or following steps (a) or (b), incubating the binding guide conjugates and half circle probes under conditions that promote ligation of half circle probes to each other, thereby producing one or more amplification target circles; and (e), following step (d), incubating the amplification target circles under conditions that promote replication of the amplification target circles, wherein replication of the amplification target circles results in the formation of tandem sequence DNA.

In some forms, the disclosed method involves a kit comprising a plurality of binding guide conjugates, wherein each binding guide conjugates comprises a specific binding molecule and a guide oligonucleotide, wherein each specific binding molecule interacts with an analyte directly or indirectly, and a plurality of half circle probes, wherein each half circle probe comprises a single-stranded DNA molecule comprising two guide complement portions, wherein each guide complement portion is complementary to at least one of the guide oligonucleotides.

In some forms the disclosed method involves (a) bringing into contact one or more analyte samples and one or more binding half circle conjugates, wherein each binding half circle conjugate comprises a specific binding molecule and a half circle probe, wherein each specific binding molecule interacts directly or indirectly with an analyte in the analyte sample, and wherein each half circle probe comprises a single-stranded DNA molecule comprising two guide complement portions, and incubating the analyte samples and the binding half circle conjugates under conditions that promote interaction of the specific binding molecules and the analytes; (b), prior to, simultaneous with, or following step (a), bringing into contact the binding half circle conjugates and one or more guide oligonucleotides, wherein each guide oligonucleotide is complementary to at least one of the guide complement portions of the half circle probes, and incubating the binding half circle conjugates and the guide oligonucleotides under conditions that promote hybridization between the guide oligonucleotides and the half circle probes; (c), following step (b) and prior to, simultaneous with, or following step (a), incubating the binding half circle conjugates and guide oligonucleotides under conditions that promote ligation of half circle probes to each other, thereby producing one or more amplification target circles; and (d), following step (c), incubating the amplification target circles under conditions that promote replication of the amplification target circles, wherein replication of the amplification target circles results in the formation of tandem sequence DNA.

In some forms, the disclosed method involves (a) bringing into contact one or more analyte samples and one or more binding guide conjugates, wherein each binding guide conjugate comprises a specific binding molecule and a guide oligonucleotide, wherein each specific binding molecule interacts directly or indirectly with an analyte in the analyte sample, and incubating the analyte samples and the binding guide conjugates under conditions that promote interaction of the specific binding molecules and the analytes; (b), prior to, simultaneous with, or following step (a), bringing into contact the binding guide conjugates and one or more half circle probes, wherein each half circle probe comprises a single-stranded DNA molecule comprising two guide complement portions, wherein each guide complement portion is complementary to at least one of the guide oligonucleotides, and incubating the binding guide conjugates and the half circle probes under conditions that promote hybridization between the guide oligonucleotides and the half circle probes; (c), following step (b) and prior to, simultaneous with, or following step (a), incubating the binding guide conjugates and half circle probes under conditions that promote ligation of half circle probes to each other, thereby producing one or more amplification target circles; and (d), following step (c), incubating the binding guide conjugates and amplification target circles under conditions that promote replication of the amplification target circles, wherein replication of the amplification target circles results in the formation of tandem sequence DNA.

In some forms, the disclosed method involves (a) bringing into contact one or more analyte samples and one or more binding guide conjugates, wherein each binding guide conjugate comprises a specific binding molecule and a guide oligonucleotide, wherein each specific binding molecule interacts directly or indirectly with an analyte in the analyte sample, and incubating the analyte samples and the binding guide conjugates under conditions that promote interaction of the specific binding molecules and the analytes; (b), prior to, simultaneous with, or following step (a), bringing into contact the binding guide conjugates and one or more half circle probes, wherein each half circle probe comprises a single-stranded DNA molecule comprising two guide complement portions, wherein each guide complement portion is complementary to at least one of the guide oligonucleotides, wherein each guide oligonucleotide is complementary to one of the guide complement portions of both half circle probes in a pair of half circle probes, wherein the guide complement portions of the same half circle probe are complementary to two different guide oligonucleotides, wherein the two different guide oligonucleotides that each are complementary to a different one of the guide complement portions of the same half circle probe constitute a pair of guide oligonucleotides, wherein the binding guide conjugates that comprise the guide oligonucleotides in a pair of guide oligonucleotides constitute a pair of binding guide conjugates, wherein one guide complement portion of each half circle probe in a pair of half circle probes is complementary to one of the guide oligonucleotides in a pair of guide oligonucleotides and the other guide complement portion of each half circle probe in the pair of half circle probes is complementary to the other guide oligonucleotide in the pair of guide oligonucleotides, wherein both half circle probes in a pair of half circle probes are hybridized to both guide oligonucleotides in a pair of guide oligonucleotides, and incubating the binding guide conjugates and the half circle probes under conditions that promote hybridization between the guide oligonucleotides and the half circle probes; and (c), following step (b) and prior to, simultaneous with, or following step (a), incubating the binding guide conjugates and half circle probes under conditions that promote ligation of half circle probes in pairs of half circle probes to each other, thereby producing one or more amplification target circles.

In some forms, the disclosed method involves (a) bringing into contact one or more analyte samples and one or more binding guide conjugates, wherein each binding guide conjugate comprises a specific binding molecule and a guide oligonucleotide, wherein each specific binding molecule interacts directly or indirectly with an analyte in the analyte sample, and incubating the analyte samples and the binding guide conjugates under conditions that promote interaction of the specific binding molecules and the analytes; (b), prior to, simultaneous with, or following step (a), bringing into contact the binding guide conjugates and one or more half circle probes, wherein each half circle probe comprises a single-stranded DNA molecule comprising two guide complement portions, wherein each guide complement portion is complementary to at least one of the guide oligonucleotides, and incubating the binding guide conjugates and the half circle probes under conditions that promote hybridization between the guide oligonucleotides and the half circle probes; and (c), following steps (a) and (b), incubating the binding guide conjugates and half circle probes under conditions that promote ligation of half circle probes to each other, thereby producing one or more amplification target circles.

Hybridization between the guide oligonucleotides and the half circle probes can result in one or more pairs of half circle probes hybridized to one or more pairs of guide oligonucleotides. One or more amplification target circles can be produced by ligation of the half circle probes in one or more of the pairs of half circle probes hybridized to pairs of guide oligonucleotides.

Each guide oligonucleotide can be complementary to one of the guide complement portions of both half circle probes in a pair of half circle probes and the guide complement portions of the same half circle probe can be complementary to two different guide oligonucleotides. The two different guide oligonucleotides that each are complementary to a different one of the guide complement portions of the same half circle probe constitute a pair of guide oligonucleotides and the binding guide conjugates that comprise the guide oligonucleotides in a pair of guide oligonucleotides constitute a pair of binding guide conjugates. One guide complement portion of each half circle probe in a pair of half circle probes can be complementary to one of the guide oligonucleotides in a pair of guide oligonucleotides and the other guide complement portion of each half circle probe in the pair of half circle probes can be complementary to the other guide oligonucleotide in the pair of guide oligonucleotides. The specific binding molecules of the binding guide conjugates in a pair of binding guide conjugates can be in close proximity when interacting with the analytes and both half circle probes in a pair of half circle probes can be hybridized to both guide oligonucleotides in a pair of guide oligonucleotides. The half circle probes in one or more pairs of half circle probes can be ligated to each other to form the amplification target circles.

Both specific binding molecules of a pair of binding guide conjugates can interact with the same analyte. Both specific binding molecules of a pair of binding guide conjugates can interact with different analytes in close proximity. Both specific binding molecules of a pair of binding guide conjugates can interact with different analytes on the same molecule. Each guide oligonucleotide can be complementary to one of the guide complement portions of both half circle probes in a different pair of half circle probes.

The binding guide conjugates brought into contact with the analyte samples can comprise a plurality of pairs of binding guide conjugates, the guide oligonucleotides of the binding guide conjugates brought into contact with the analyte samples can comprise a plurality of pairs of guide oligonucleotides, and the half circle probes brought into contact with the binding guide conjugates can comprise a plurality of pairs of half circle probes.

Both specific binding molecules of each pair of binding guide conjugates can interact with the same analyte. Both specific binding molecules of each pair of binding guide conjugates can interact with different analytes in close proximity. Both specific binding molecules of each pair of binding guide conjugates can interact with different analytes on the same molecule. The specific binding molecules of each pair of binding guide conjugates do not interact with the same analyte as the specific binding molecules of any other pair of binding guide conjugates. The specific binding molecules of one or more pairs of binding guide conjugates can interact with the same analyte as the specific binding molecules of another pair of binding guide conjugates. The half circle probes of each pair of half circle probes can be hybridized to only one of the pairs of guide oligonucleotides. The half circle probes of each pair of half circle probes can be hybridized to one or more of the pairs of guide oligonucleotides. The half circle probes of each pair of half circle probes can be hybridized to only one of the pairs of guide oligonucleotides and the other half circle probe of each pair of half circle probes can be hybridized to a plurality of the pairs of guide oligonucleotides. The half circle probes of each pair of half circle probes can be hybridized to only one of the pairs of guide oligonucleotides and the other half circle probe of each pair of half circle probes can be hybridized to all of the pairs of guide oligonucleotides. A plurality of amplification target circles can be produced by ligation of a plurality of the pairs of half circle probes.

The binding guide conjugates brought into contact with the analyte samples can comprise a pair of binding guide conjugates, the guide oligonucleotides of the binding guide conjugates brought into contact with the analyte samples can comprise a pair of guide oligonucleotides, and the half circle probes brought into contact with the binding guide conjugates can comprise a pair of half circle probes. The half circle probes in a pair of half circle probes can be sequentially brought into contact with the binding guide conjugates.

Each guide oligonucleotide can be complementary to one of the guide complement portions of both half circle probes in a pair of half circle probes and the guide complement portions of the same half circle probe can be complementary to two different guide oligonucleotides. The two different guide oligonucleotides that each are complementary to a different one of the guide complement portions of the same half circle probe constitute a pair of guide oligonucleotides and the binding guide conjugates that comprise the guide oligonucleotides in a pair of guide oligonucleotides constitute a pair of binding guide conjugates. One guide complement portion of each half circle probe in a pair of half circle probes can be complementary to one of the guide oligonucleotides in a pair of guide oligonucleotides and the other guide complement portion of each half circle probe in the pair of half circle probes can be complementary to the other guide oligonucleotide in the pair of guide oligonucleotides. The specific binding molecules can interact with different analytes, the specific binding molecule of one of the binding guide conjugates in a pair of binding guide conjugates can interact with an analyte in close proximity to the analyte that interacts with the specific binding molecule of the other binding guide conjugate in the pair of binding guide conjugates, and both half circle probes in a pair of half circle probes can be hybridized to both guide oligonucleotides in a pair of guide oligonucleotides. The half circle probes in a pair of half circle probes can be ligated to each other to form an amplification target circle.

The disclosed method can further involve incubating the amplification target circles under conditions that promote replication of the amplification target circles, and replication of the amplification target circles can result in the formation of tandem sequence DNA.

The disclosed method can further involve bringing into contact at least one of the analyte samples and one or more analyte capture agents, wherein each analyte capture agent can interact with an analyte directly or indirectly and at least one analyte, if present in the analyte sample, can interact with at least one analyte capture agent. At least one analyte capture agent can be associated with a solid support and analytes that interact with the analyte capture agent associated with a solid support can become associated with the solid support. Each of the analyte capture agents can be located in a different predefined region of the solid support. The location of tandem sequence DNA on the solid support can indicate the presence in the analyte sample of the analyte corresponding to the analyte capture agent at that location of the solid support.

The disclosed method can further involve detecting the tandem sequence DNA. Detection of tandem sequence DNA can indicate the presence of the corresponding analytes. Detection of the tandem sequence DNA can be accomplished by mixing a set of detection probes with the tandem sequence DNA under conditions that promote hybridization between the tandem sequence DNA and the detection probes. A plurality of different tandem sequence DNAs can be detected separately and simultaneously via multiplex detection. The set of detection probes can be labeled using combinatorial multicolor coding.

The disclosed method can further involve bringing into contact a secondary DNA strand displacement primer and the tandem sequence DNA and incubating under conditions that promote (i) hybridization between the tandem sequence DNA and the secondary DNA strand displacement primer, and (ii) replication of the tandem sequence DNA, wherein replication of the tandem sequence DNA results in the formation of secondary tandem sequence DNA.

Analytes can be separated from the analyte samples. Analytes can be separated from the analyte sample by bringing into contact at least one of the analyte samples and one or more analyte capture agents, wherein each analyte capture agent can interact with an analyte directly or indirectly, and at least one analyte, if present in the analyte sample, can interact with at least one analyte capture agent, and separating analyte capture agents from the analyte samples, thus separating analytes from the analyte samples.

A plurality of binding guide conjugates can be brought into contact with the one or more analyte samples. A plurality of analyte samples can be brought into contact with the one or more binding guide conjugates. At least one of the analytes can be a protein or peptide. At least one of the analytes can be a lipid, glycolipid, or proteoglycan. At least one of the analytes can be from a human source. At least one of the analytes can be from a non-human source. None of the analytes can be nucleic acids. At least one of the analyte samples and one or more analyte capture agents can be brought into contact, each analyte capture agent can interact with an analyte directly or indirectly, and at least one analyte, if present in the analyte sample, can interact with at least one analyte capture agent.

The analyte capture agents can be separated from the analyte samples, thus separating analytes from the analyte samples. At least one analyte capture agent can be associated with a solid support and analytes that interact with the analyte capture agent associated with a solid support can be become associated with the solid support. Each of the analyte capture agents can be located in a different predefined region of the solid support. The distance between the different predefined regions of the solid support can be fixed. The solid support can comprise thin film, membrane, bottles, dishes, fibers, woven fibers, shaped polymers, particles, beads, microparticles, or a combination. The distance between at least two of the different predefined regions of the solid support can be variable. The solid support can comprise at least one thin film, membrane, bottle, dish, fiber, woven fiber, shaped polymer, particle, bead, or microparticle. The solid support can comprise at least two thin films, membranes, bottles, dishes, fibers, woven fibers, shaped polymers, particles, beads, microparticles, or a combination. The solid support can comprise a plurality of analyte capture agents located in a plurality of different predefined regions of the solid support, wherein the analyte capture agents collectively correspond to a plurality of analytes. The solid support can comprise thin film, membrane, bottles, dishes, fibers, woven fibers, shaped polymers, particles, beads, microparticles, or a combination. The solid support can comprise acrylamide, agarose, cellulose, nitrocellulose, glass, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, polypropylfumerate, collagen, glycosaminoglycans, or polyamino acids. The solid support can be porous.

The disclosed method can further involve bringing into contact at least one of the analyte samples and at least one of the binding guide conjugates with at least one accessory molecule and the accessory molecule can affect the interaction of at least one of the analytes and at least one of the specific binding molecules or at least one of the analyte capture agents.

The accessory molecule can be brought into contact with at least one of the analyte samples, at least one of the binding guide conjugates, or both. At least one analyte capture agent can be associated with a solid support and the accessory molecule can be associated with the solid support. The accessory molecule can be associated with the solid support by bringing the accessory molecule into contact with the solid support. The accessory molecule can be a protein kinase, a protein phosphatase, an enzyme, or a compound. The accessory molecule can be a molecule of interest, one or more of the analytes can be test molecules, and interactions of the test molecules with the molecule of interest can be detected.

At least one of the analytes can be a molecule of interest, the accessory molecule can be a test molecule, and interactions of the test molecule with the molecule of interest can be detected. At least one of the analyte capture agents can be a molecule of interest, one or more of the analytes can be test molecules, and interactions of the test molecules with the molecule of interest can be detected. At least one of the analytes can be a molecule of interest, one or more of the analyte capture agents can be test molecules, and interactions of the test molecules with the molecule of interest can be detected.

At least one of the specific binding molecules can be an antibody specific for at least one of the analytes. At least one of the specific binding molecules can be a molecule that specifically binds to at least one of the analytes. At least one of the specific binding molecules can be a molecule that specifically binds to at least one of the analytes in combination with an accessory molecule.

At least one accessory molecule can be brought into contact with at least one of the analyte samples and at least one of the binding guide conjugates and the accessory molecule can affect the interaction of at least one of the analytes and at least one of the specific binding molecules or at least one of the analyte capture agents. The accessory molecule can compete with the interaction of at least one of the specific binding molecules or at least one of the analyte capture agents. The accessory molecule can be an analog of at least one of the analytes. The accessory molecule can facilitate the interaction of at least one of the specific binding molecules or at least one of the analyte capture agents. The accessory molecule can be brought into contact with at least one of the analyte samples, at least one of the binding guide conjugates, or both. The accessory molecule can be a protein kinase, a protein phosphatase, an enzyme, or a compound. The accessory molecule can be at least 20% pure. The accessory molecule can be at least 50% pure. The accessory molecule can be at least 80% pure. The accessory molecule can be at least 90% pure.

At least one of the analytes can be associated with a solid support. Each of the analytes associated with the solid support can be associated with the solid support in a different predefined region. At least one of the analytes associated with the solid support can be associated with the solid support indirectly. The analytes associated with the solid support can interact with analyte capture agents and the analyte capture agents can be associated with the solid support, thereby indirectly associating the analytes with the solid support.

At least one specific binding molecule can interact with at least one analyte indirectly. The analyte can interact with an analyte capture agent and the specific binding molecule can interact with the analyte capture agent, thereby indirectly associating the specific binding molecule with the analyte.

At least one of the analytes can be a modified form of another analyte, the specific binding molecule of at least one of the binding guide conjugates can interact, directly or indirectly, with the analyte that can be a modified form of the other analyte, and the specific binding molecule of another binding guide conjugate can interact, directly or indirectly, with the other analyte. The analytes can be proteins and the modification of the modified form of the other analyte can be a post-translational modification. The modification can be phosphorylation or glycosylation.

The binding guide conjugates can be at least 20% pure. The binding guide conjugates can be at least 50% pure. The binding guide conjugates can be at least 80% pure. The binding guide conjugates can be at least 90% pure. The binding guide conjugates can include one or more first binding guide conjugates and one or more second binding guide conjugates and the half circle probes can include one or more first half circle probes and one or more second half circle probes. Each first binding guide conjugate can correspond to at least one of the second binding guide conjugates and the specific binding molecules of the first binding guide conjugates can interact with the same analyte as the specific binding molecule of the second binding guide conjugate to which the first binding guide conjugate corresponds. One guide compliment portion of the first half circle probes can hybridize to the guide oligonucleotide of the first binding guide conjugate and another guide compliment portion of the first half circle probe can hybridize to the guide oligonucleotide of the second binding guide conjugate. One guide compliment portion of the second half circle probe can hybridize to the guide oligonucleotide of the first binding guide conjugate and another guide compliment portion of the second half circle probe can hybridize to the guide oligonucleotide of the second binding guide conjugate. The first and second half circle probes can be ligated together to form the amplification target circle. The first and second half circle probes can be sequentially brought into contact with the binding guide conjugates.

The analyte samples can include two or more analytes in close proximity, the binding guide conjugates can include one or more first binding guide conjugates and one or more second binding guide conjugates, and the half circle probes can include one or more first half circle probes and one or more second half circle probes. Each first binding guide conjugate can correspond to at least one of the second binding guide conjugates and the specific binding molecule of the first binding guide conjugates can interact with an analyte in close proximity to the analyte that the specific binding molecule of the second binding guide conjugate to which the first binding conjugate interacts with. One guide compliment portion of the first half circle probe can hybridize to the guide oligonucleotide of the first binding guide conjugate and another guide compliment portion of the first half circle probe can hybridize to the guide oligonucleotide of the second binding guide conjugate. One guide compliment portion of the second half circle probe can hybridize to the guide oligonucleotide of the first binding guide conjugate and another guide compliment portion of the second half circle probe can hybridize to the guide oligonucleotide of the second binding guide conjugate. The first and second half circle probes can be ligated together to form the amplification target circle.

Two guide complement portions of two different half circle probes can hybridize to the guide oligonucleotides and one of the guide complement portions can have a 5′ end and the other guide complement portion can have a 3′ end. The 5′ end and the 3′ end can be immediately adjacent when the two guide complement portions are hybridized to the guide oligonucleotide. The 5′ end and the 3′ end can be separated by a central region when the two guide complement portions are hybridized to the guide oligonucleotide. The disclosed method can further involve bringing one or more gap oligonucleotides into contact with the binding guide conjugates, wherein each gap oligonucleotide can comprise a single-stranded, linear DNA molecule, wherein each gap oligonucleotide can be complementary to all or a portion of the central region, and wherein the gap oligonucleotides complementary to the central region can be incorporated into the amplification target circles.

Each array can comprises a set of analyte capture agents and each analyte capture agent can be immobilized on a solid support in a different predefined region of the solid support. The distance between the different predefined regions of the solid support can be fixed. The solid support can comprise thin film, membrane, bottles, dishes, fibers, woven fibers, shaped polymers, particles, beads, microparticles, or a combination. The distance between at least two of the different predefined regions of the solid support can be variable. The analyte capture agents can be immobilized to the solid support at a density exceeding 400 different analyte capture agents per cubic centimeter. The analyte capture agents can be peptides. Each of the different peptides can be at least 4 amino acids in length. Each different peptide can be from about 4 to about 20 amino acids in length. Each different peptide can be at least 10 amino acids in length. Each different peptide can be at least 20 amino acids in length. At least one array can comprise at least 1,000 different analyte capture agents immobilized on the solid support. At least one array can comprise at least 10,000 different analyte capture agents immobilized on the solid support. At least one array can comprise at least 100,000 different analyte capture agents immobilized on the solid support. At least one array can comprise at least 1,000,000 different analyte capture agents immobilized on the solid support. Each of the different predefined regions can be physically separated from each other of the different regions.

The solid support can comprise thin film, membrane, bottles, dishes, fibers, woven fibers, shaped polymers, particles, beads, microparticles, or a combination. The solid support can comprise acrylamide, agarose, cellulose, nitrocellulose, glass, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, polypropylfumerate, collagen, glycosaminoglycans, or polyamino acids. The solid support can be porous. The analyte capture agents in the different predefined regions can be at least 20% pure. The analyte capture agents in the different predefined regions can be at least 50% pure. The analyte capture agents in the different predefined regions can be at least 80% pure. The analyte capture agents in the different predefined regions can be at least 90% pure.

All of the analytes can be modified by associating a modifying group to the analytes, the modifying group can be the same for all of the analytes, and all of the specific binding molecules can interact with the modifying group.

The kit can further involve a plurality of analyte capture agents and each analyte capture agent can interact with an analyte directly or indirectly. The analyte capture agents can be associated with a solid support. Each guide oligonucleotide can be complementary to one of the guide complement portions of both half circle probes in a pair of half circle probes and the guide complement portions of the same half circle probe can be complementary to two different guide oligonucleotides. The two different guide oligonucleotides that each are complementary to a different one of the guide complement portions of the same half circle probe constitute a pair of guide oligonucleotides and the binding guide conjugates that comprise the guide oligonucleotides in a pair of guide oligonucleotides constitute a pair of binding guide conjugates. One guide complement portion of each half circle probe in a pair of half circle probes can be complementary to one of the guide oligonucleotides in a pair of guide oligonucleotides and the other guide complement portion of each half circle probe in the pair of half circle probes can be complementary to the other guide oligonucleotide in the pair of guide oligonucleotides. The specific binding molecules of the binding guide conjugates in a pair of binding guide conjugates can interact with the same analyte and both half circle probes in a pair of half circle probes can be hybridized to both guide oligonucleotides in a pair of guide oligonucleotides. Each guide oligonucleotide can be complementary to one of the guide complement portions of both half circle probes in a different pair of half circle probes. The binding guide conjugates can comprise a plurality of pairs of binding guide conjugates, the guide oligonucleotides of the binding guide conjugates can comprise a plurality of pairs of guide oligonucleotides, and the half circle probes can comprise a plurality of pairs of half circle probes. The binding guide conjugates can comprise a pair of binding guide conjugates, the guide oligonucleotides of the binding guide conjugates can comprise a pair of guide oligonucleotides, and the half circle probes can comprise a pair of half circle probes. The amplification target circles can be disassociated from the specific binding molecules. The amplification target circles can be disassociated from the specific binding molecules by cleaving covalent bonds. The amplification target circles can be disassociated from the specific binding molecules by treating the amplification target circles with a reducing agent. The amplification target circles can be disassociated from the specific binding molecules by heating the amplification target circles. The half circle probes can be tethered to the specific binding molecules. The half circle probes can be covalently coupled to the specific binding molecules. The half circle probes can be covalently coupled to the specific binding molecules via a cleavable linker.

### EXAMPLES

### A. Example 1

ATC formation by Double Ligation

Two half circle probes (HCP1 and HCP2) and two guide oligonucleotides (G1 and G2) were prepared to evaluate double ligation. A single ligation model using an open circle probe (OCP) and target sequence (G5) was also prepared to be used as a control. The half circle probes and guide oligonucleotides were synthesized based on the sequence of the open circle probe H63D 5901/1704-2 mut#5. The 78 nucleotide sequence of H63D was “cut” exactly in half to give the 39 nucleotides each half circle probes. The two guide oligonucleotides contained 10 base sequence complementary to the ends of each half circle probe. The sequence of the guide oligonucleotides (G1 and G2) and half circle probes (HCP1 and HCP2) based on the H63D 5901/1704-2 mut#5 and the open circle probe (OCP) and target sequence (G5) are as follows:

| | |
|---|---|
| G1, 5′-C6Thiol/AAAAAAAAAAAAAAAAAAAAAAAAAAAAAAGTTCTAT | SEQ ID NO:1 |
| GATGATGAGAGTC-3′: | |
| G2, 5′-/C6Thiol/AAAAAAAAAAAAAAAAAAAAAAAAAAAAAACGCGAC | SEQ ID NO:2 |
| GGCCGATAACAGAG-3′: | |
| OCP1, 5′-/phosphate/ATCATAGAACACGAACAGCTGGTCATCTGCTCTGT | SEQ ID NO:3 |
| TATC-3′: | |
| OCP2, 5′-/phosphate/GGCCGTCGCGCAGACACGATAGATGAGGCGACTC | SEQ ID NO:4 |
| TCATC-3′: | |
| G5, 5′-TCTATGATGATGAGAGTCGC-3′: | SEQ ID NO:5 |
| OCP, 5′-/phosphate/ATCATAGAACACGAACAGCTGGTCATCTGCTCTGTT | SEQ ID NO:6 |
| ATCGGCCGTCGCGCAGACACGATAGATGAGGCGACTCTCATC-3′: | |

All reactions were treated with T4 DNA ligase (NEB) and were performed at room temperature for 20 minutes. Exonuclease treatment was for 1 hour at 37° C. Ligation products were resolved by electrophoresis on a 15% TBE-UREA gel and stained with Gel Star.

When both half circle probes and both guides oligonucleotides were incubated in the presence of T4 DNA ligase a number of ligation products were observed following gel electrophoresis. When the ligation products were digested with a mixture of Exonuclease I and Exonuclease III the major exonuclease resistant products migrated with mobilities identical to that of single and double circle produced by single ligation of half circle probes. The amount of double circle produced by double ligation was significantly less than that produced by conventional single ligation. Similar results were obtained on half circle probes based on the sequence of OCP 1822 ocT (8-8).

### B. Example 2

ATC Formation on Binding Guide Conjugates

Example 1 was repeated using binding guide conjugates where guide oligonucleotides (G1 and G2) conjugated to anti-biotin monoclonal antibodies. When the ligation products were digested with exonuclease the major exonuclease resistant products migrated with mobilities identical to that of single and double circle produced by single ligation of half circle probes. Conjugation of guide oligonucleotides to the binding guide conjugates did not greatly reduce the amount of single circle formed and gave similarly reduced levels of double circle when compared to free guide oligonucleotides.

These examples clearly show that the double ligation method can result in small amplification target circle formation. ATC preparations produced by this method were more homogeneous containing less “tandem circles” than preparations produced by a single ligation method. Thus in addition to ATC formation tied to a diagnostic test the double ligation method may also be used for large-scale circle production.

Double ligation has certain advantages over single ligation for ATC production. For example, synthesis of ˜40 mer HCPs can be significantly more efficient than ˜80 mer OCPs, which can more than offset the cost of synthesizing the additional guide. Also, unlike 80 mers, quality control on 40 mers can be performed by mass spectrometry. Semi-quantitative quality control by this method would be advantageous. Further, more homogeneous circle preparations could minimize variability between lots and reduce possible artifacts resulting from multiple priming of tandem circles (e.g., replication fork displacement).

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a primer” includes a plurality of such primers, reference to “the primer” is a reference to one or more primers and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are specifically incorporated by reference. Nothing herein is to be construed as an admission that the present invention is not entitled to antedate such disclosure by virtue of prior invention.

Those skilled in the art will recognize, or be able to ascertain using no more than routine experimentation, many equivalents to the specific embodiments of the method and compositions described herein. Such equivalents are intended to be encompassed by the following claims.

We claim:

1. A method comprising:
(a) bringing into contact one or more analyte samples and one or more binding guide conjugates, wherein each binding guide conjugate comprises a specific binding molecule and a guide oligonucleotide, wherein each specific binding molecule interacts directly or indirectly with an analyte in the analyte sample, and
incubating the analyte samples and the binding guide conjugates under conditions that promote interaction of the specific binding molecules and the analytes,
(b) prior to, simultaneous with, or following step (a), bringing into contact the binding guide conjugates and one or more half circle probes, wherein each half circle probe comprises a single-stranded DNA molecule comprising two guide complement portions, wherein each guide complement portion is complementary to one of the guide oligonucleotides, and
incubating the binding guide conjugates and the half circle probes under conditions that promote hybridization between the guide oligonucleotides and the half circle probes,
(c) following step (b) and prior to, simultaneous with, or following step (a), incubating the binding guide conjugates and half circle probes under conditions that promote ligation of half circle probes, thereby producing one or more amplification target circles.

2. The method of claim 1, wherein hybridization between the guide oligonucleotides and the half circle probes results in one or more pairs of half circle probes hybridized to one or more pairs of guide oligonucleotides.

3. The method of claim 2, wherein one or more amplification target circles are produced by ligation of the half circle probes in one or more of the pairs of half circle probes hybridized to pairs of guide oligonucleotides.

4. The method of claim 1, wherein each guide oligonucleotide is complementary to one of the guide complement portions of both half circle probes in a pair of half circle probes, wherein the guide complement portions of the same half circle probe are complementary to two different guide oligonucleotides,
wherein the two different guide oligonucleotides that each are complementary to a different one of the guide complement portions of the same half circle probe constitute a pair of guide oligonucleotides, wherein the binding guide conjugates that comprise the guide oligonucleotides in a pair of guide oligonucleotides constitute a pair of binding guide conjugates,
wherein one guide complement portion of each half circle probe in a pair of half circle probes is complementary to one of the guide oligonucleotides in a pair of guide oligonucleotides and the other guide complement portion of each half circle probe in the pair of half circle probes is complementary to the other guide oligonucleotide in the pair of guide oligonucleotides,
wherein the specific binding molecules of the binding guide conjugates in a pair of binding guide conjugates are in close proximity when interacting with the analytes, wherein both half circle probes in a pair of half circle probes are hybridized to both guide oligonucleotides in a pair of guide oligonucleotides, and
wherein the half circle probes in one or more pairs of half circle probes are ligated to each other to form the amplification target circles.

5. The method of claim 4, wherein both specific binding molecules of a pair of binding guide conjugates interact with the same analyte.

6. The method of claim 4, wherein both specific binding molecules of a pair of binding guide conjugates interact with different analytes in close proximity.

7. The method of claim 4, wherein both specific binding molecules of a pair of binding guide conjugates interact with different analytes on the same molecule.

8. The method of claim 4, wherein each guide oligonucleotide is complementary to one of the guide complement portions of both half circle probes in a different pair of half circle probes.

9. The method of claim 4, wherein the binding guide conjugates brought into contact with the analyte samples comprise a plurality of pairs of binding guide conjugates, wherein the guide oligonucleotides of the binding guide conjugates brought into contact with the analyte samples comprise a plurality of pairs of guide oligonucleotides, and wherein the half circle probes brought into contact with the binding guide conjugates comprise a plurality of pairs of half circle probes.

10. The method of claim 9, wherein both specific binding molecules of each pair of binding guide conjugates interact with the same analyte.

11. The method of claim 9, wherein both specific binding molecules of each pair of binding guide conjugates interact with different analytes in close proximity.

12. The method of claim 9, wherein both specific binding molecules of each pair of binding guide conjugates interact with different analytes on the same molecule.

13. The method of claim 9, wherein the specific binding molecules of each pair of binding guide conjugates do not interact with the same analyte as the specific binding molecules of any other pair binding guide conjugates.

14. The method of claim 9, wherein the specific binding molecules of one or more pairs of binding guide conjugates interact with the same analyte as the specific binding molecules of another pair binding guide conjugates.

15. The method of claim 9, wherein the half circle probes of each pair of half circle probes are hybridized to only one of the pairs of guide oligonucleotides.

16. The method of claim 9, wherein the half circle probes of each pair of half circle probes are hybridized to one or more of the pairs of guide oligonucleotides.

17. The method of claim 9, wherein one of the half circle probes of each pair of half circle probes is hybridized to only one of the pairs of guide oligonucleotides and the other half circle probe of each pair of half circle probes is hybridized to a plurality of the pairs of guide oligonucleotides.

18. The method of claim 9, wherein one of the half circle probes of each pair of half circle probes is hybridized to only one of the pairs of guide oligonucleotides and the other half circle probe of each pair of half circle probes is hybridized to all of the pairs of guide oligonucleotides.

19. The method of claim 9, wherein a plurality of amplification target circles are produced by ligation of a plurality of the pairs of half circle probes.

20. The method of claim 4, wherein the binding guide conjugates brought into contact with the analyte samples comprise a pair of binding guide conjugates, wherein the guide oligonucleotides of the binding guide conjugates brought into contact with the analyte samples comprise a pair of guide oligonucleotides, and wherein the half circle probes brought into contact with the binding guide conjugates comprise a pair of half circle probes.

21. The method of claim 4, wherein the half circle probes in a pair of half circle probes are sequentially brought into contact with the binding guide conjugates.

22. The method of claim 1, wherein each guide oligonucleotide is complementary to one of the guide complement portions of both half circle probes in a pair of half circle probes, wherein the guide complement portions of the same half circle probe are complementary to two different guide oligonucleotides,
wherein the two different guide oligonucleotides that each are complementary to a different one of the guide complement portions of the same half circle probe constitute a pair of guide oligonucleotides, wherein the binding guide conjugates that comprise the guide oligonucleotides in a pair of guide oligonucleotides constitute a pair of binding guide conjugates,
wherein one guide complement portion of each half circle probe in a pair of half circle probes is complementary to one of the guide oligonucleotides in a pair of guide oligonucleotides and the other guide complement portion of each half circle probe in the pair of half circle probes is complementary to the other guide oligonucleotide in the pair of guide oligonucleotides,
wherein the specific binding molecules interact with different analytes, wherein the specific binding molecule of one of the binding guide conjugates in a pair of binding guide conjugates interacts with an analyte in close proximity to the analyte that interacts with the specific binding molecule of the other binding guide conjugate in the pair of binding guide conjugates, wherein both half circle probes in a pair of half circle probes are hybridized to both guide oligonucleotides in a pair of guide oligonucleotides, and
wherein the half circle probes in a pair of half circle probes are ligated to each other to form an amplification target circle.

23. The method of claim 1, wherein the method further comprises, following step (c),
(d) incubating the amplification target circles under conditions that promote replication of the amplification target circles, wherein replication of the amplification target circles results in the formation of tandem sequence DNA.

24. The method of claim 23, wherein the method further comprises bringing into contact at least one of the analyte samples and one or more analyte capture agents, wherein each analyte capture agent interacts with an analyte directly or indirectly, wherein at least one analyte, if present in the analyte sample, interacts with at least one analyte capture agent,
wherein at least one analyte capture agent is associated with a solid support, wherein analytes that interact with the analyte capture agent associated with a solid support become associated with the solid support,
wherein each of the analyte capture agents is located in a different predefined region of the solid support, and
wherein the location of tandem sequence DNA on the solid support indicates the presence in the analyte sample of the analyte corresponding to the analyte capture agent at that location of the solid support.

25. The method of claim 23, wherein the method further comprises detecting the tandem sequence DNA.

26. The method of claim 25, wherein detection of tandem sequence DNA indicates the presence of the corresponding analytes.

27. The method of claim 25, wherein detection of the tandem sequence DNA is accomplished by mixing a set of detection probes with the tandem sequence DNA under conditions that promote hybridization between the tandem sequence DNA and the detection probes.

28. The method of claim 27, wherein a plurality of different tandem sequence DNAs are detected separately and simultaneously via multiplex detection.

29. The method of claim 28, wherein the set of detection probes is labeled using combinatorial multicolor coding.

30. The method of claim 23, further comprising, simultaneous with, or following, step (d),
bringing into contact a secondary DNA strand displacement primer and the tandem sequence DNA, and incubating under conditions that promote (i) hybridization between the tandem sequence DNA and the secondary DNA strand displacement primer, and (ii) replication of the tandem sequence DNA, wherein replication of the tandem sequence DNA results in the formation of secondary tandem sequence DNA.

31. The method of claim 1, wherein prior to, simultaneous with, or following steps (a), (b), or (c) the analytes are separated from the analyte samples.

32. The method of claim 31, wherein the analytes are separated from the analyte sample by bringing into contact at least one of the analyte samples and one or more analyte capture agents, wherein each analyte capture agent interacts with an analyte directly or indirectly, wherein at least one analyte, if present in the analyte sample, interacts with at least one analyte capture agent, and separating analyte capture agents from the analyte samples, thus separating analytes from the analyte samples.

33. The method of claim 1, wherein a plurality of binding guide conjugates are brought into contact with the one or more analyte samples.

34. The method of claim 1, wherein a plurality of analyte samples are brought into contact with the one or more binding guide conjugates.

35. The method of claim 1, wherein at least one of the analytes is a protein or peptide.

36. The method of claim 1, wherein at least one of the analytes is a lipid, glycolipid, or proteoglycan.

37. The method of claim 1, wherein at least one of the analytes is from a human source.

38. The method of claim 1, wherein at least one of the analytes is from a non-human source.

39. The method of claim 1, wherein none of the analytes are nucleic acids.

40. The method of claim 1, wherein at least one of the analyte samples and one or more analyte capture agents are brought into contact, wherein each analyte capture agent interacts with an analyte directly or indirectly, and wherein at least one analyte, if present in the analyte sample, interacts with at least one analyte capture agent.

41. The method of claim 40, wherein the analyte capture agents are separated from the analyte samples, thus separating analytes from the analyte samples.

42. The method of claim 40, wherein at least one analyte capture agent is associated with a solid support, and wherein analytes that interact with the analyte capture agent associated with a solid support become associated with the solid support.

43. The method of claim 42, wherein each of the analyte capture agents is located in a different predefined region of the solid support.

44. The method of claim 43, wherein the distance between the different predefined regions of the solid support is fixed.

45. The method of claim 44, wherein the solid support comprises thin film, membrane, bottles, dishes, fibers, woven fibers, shaped polymers, particles, beads, microparticles, or a combination.

46. The method of claim 43, wherein the distance between at least two of the different predefined regions of the solid support is variable.

47. The method of claim 46, wherein the solid support comprises at least one thin film, membrane, bottle, dish, fiber, woven fiber, shaped polymer, particle, bead, or microparticle.

48. The method of claim 47, wherein the solid support comprises at least two thin films, membranes, bottles, dishes, fibers, woven fibers, shaped polymers, particles, beads, microparticles, or a combination.

49. The method of claim 42, wherein the solid support comprises a plurality of analyte capture agents located in a plurality of different predefined regions of the solid support, wherein the analyte capture agents collectively correspond to a plurality of analytes.

50. The method of claim 42, wherein the solid support comprises thin film, membrane, bottles, dishes, fibers, woven fibers, shaped polymers, particles, beads, microparticles, or a combination.

51. The method of claim 42, wherein the solid support comprises acrylamide, agarose, cellulose, nitrocellulose, glass, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, polypropylfumerate, collagen, glycosaminoglycans, or polyamino acids.

52. The method of claim 42, wherein the solid support is porous.

53. The method of claim 40, further comprising bringing into contact at least one of the analyte samples and at least one of the binding guide conjugates with at least one accessory molecule, and wherein the accessory molecule affects the interaction of at least one of the analytes and at least one of the specific binding molecules or at least one of the analyte capture agents.

54. The method of claim 53, wherein the accessory molecule is brought into contact with at least one of the analyte samples, at least one of the binding guide conjugates, or both, prior to, simultaneous with, or following step (a).

55. The method of claim 53, wherein at least one analyte capture agent is associated with a solid support, and wherein the accessory molecule is associated with the solid support.

56. The method of claim 55, wherein the accessory molecule is associated with the solid support by bringing the accessory molecule into contact with the solid support prior to, simultaneous with, or following step (a).

57. The method of claim 53, wherein the accessory molecule is a protein kinase, a protein phosphatase, an enzyme, or a compound.

58. The method of claim 53, wherein the accessory molecule is a molecule of interest, wherein one or more of the analytes are test molecules, and wherein interactions of the test molecules with the molecule of interest are detected.

59. The method of claim 53, wherein at least one of the analytes is a molecule of interest, wherein the accessory molecule is a test molecule, and wherein interactions of the test molecule with the molecule of interest are detected.

60. The method of claim 40, wherein at least one of the analyte capture agents is a molecule of interest, wherein one or more of the analytes are test molecules, and wherein interactions of the test molecules with the molecule of interest are detected.

61. The method of claim 40, wherein at least one of the analytes is a molecule of interest, wherein one or more of the analyte capture agents are test molecules, and wherein interactions of the test molecules with the molecule of interest are detected.

62. The method of claim 1, wherein at least one of the specific binding molecules is an antibody specific for at least one of the analytes.

63. The method of claim 1, wherein at least one of the specific binding molecules is a molecule that specifically binds to at least one of the analytes.

64. The method of claim 1, wherein at least one of the specific binding molecules is a molecule that specifically binds to at least one of the analytes in combination with an accessory molecule.

65. The method of claim 1, wherein at least one accessory molecule is brought into contact with at least one of the analyte samples and at least one of the binding guide conjugates, and wherein the accessory molecule affects the interaction of at least one of the analytes and at least one of the specific binding molecules or at least one of the analyte capture agents.

66. The method of claim 65, wherein the accessory molecule competes with the interaction of at least one of the specific binding molecules or at least one of the analyte capture agents.

67. The method of claim 66, wherein the accessory molecule is an analog of at least one of the analytes.

68. The method of claim 65, wherein the accessory molecule facilitates the interaction of at least one of the specific binding molecules or at least one of the analyte capture agents.

69. The method of claim 65, wherein the accessory molecule is brought into contact with at least one of the analyte samples, at least one of the binding guide conjugates, or both, prior to, simultaneous with, or following step (a).

70. The method of claim 65, wherein the accessory molecule is a protein kinase, a protein phosphatase, an enzyme, or a compound.

71. The method of claim 65, wherein the accessory molecule is at least 20% pure.

72. The method of claim 65, wherein the accessory molecule is at least 50% pure.

73. The method of claim 65, wherein the accessory molecule is at least 80% pure.

74. The method of claim 65, wherein the accessory molecule is at least 90% pure.

75. The method of claim 1, wherein at least one of the analytes is associated with a solid support.

76. The method of claim 75, wherein each of the analytes associated with the solid support is associated with the solid support in a different predefined region.

77. The method of claim 75, wherein at least one of the analytes associated with the solid support is associated with the solid support indirectly.

78. The method of claim 77, wherein the analytes associated with the solid support interact with analyte capture agents, and wherein the analyte capture agents are associated with the solid support thereby indirectly associating the analytes with the solid support.

79. The method of claim 1, wherein at least one specific binding molecule interacts with at least one analyte indirectly.

80. The method of claim 79, wherein the analyte interacts with an analyte capture agent, and wherein the specific binding molecule interacts with the analyte capture agent thereby indirectly associating the specific binding molecule with the analyte.

81. The method of claim 1, wherein at least one of the analytes is a modified form of another analyte, wherein the specific binding molecule of at least one of the binding guide conjugates interacts, directly or indirectly, with the analyte that is a modified form of the other analyte, and wherein the specific binding molecule of another binding guide conjugate interacts, directly or indirectly, with the other analyte.

82. The method of claim 81, wherein the analytes are proteins, wherein the modification of the modified form of the other analyte is a post-translational modification.

83. The method of claim 82, wherein the modification is phosphorylation or glycosylation.

84. The method of claim 1, wherein the binding guide conjugates are at least 20% pure.

85. The method of claim 1, wherein the binding guide conjugates are at least 50% pure.

86. The method of claim 1, wherein the binding guide conjugates are at least 80% pure.

87. The method of claim 1, wherein the binding guide conjugates are at least 90% pure.

88. The method of claim 1, wherein the binding guide conjugates include one or more first binding guide conjugates and one or more second binding guide conjugates, wherein the half circle probes include one or more first half circle probes and one or more second half circle probes,
wherein each first binding guide conjugate corresponds to at least one of the second binding guide conjugates, wherein the specific binding molecules of the first binding guide conjugates interact with the same analyte as the specific binding molecule of the second binding guide conjugate to which the first binding guide conjugate corresponds,
wherein one guide compliment portion of the first half circle probes hybridizes to the guide oligonucleotide of the first binding guide conjugate and another guide compliment portion of the first half circle probe hybridizes to the guide oligonucleotide of the second binding guide conjugate,
wherein one guide compliment portion of the second half circle probe hybridizes to the guide oligonucleotide of the first binding guide conjugate and another guide compliment portion of the second half circle probe hybridizes to the guide oligonucleotide of the second binding guide conjugate, and
wherein the first and second half circle probes are ligated together to form the amplification target circle.

89. The method of claim 88, wherein the first and second half circle probes are sequentially brought into contact with the binding guide conjugates.

90. The method of claim 1, wherein the analyte samples include two or more analytes in close proximity, wherein the binding guide conjugates include one or more first binding guide conjugates and one or more second binding guide conjugates, wherein the half circle probes include one or more first half circle probes and one or more second half circle probes,
wherein each first binding guide conjugate corresponds to at least one of the second binding guide conjugates, wherein the specific binding molecule of the first binding guide conjugates interact with an analyte in close proximity to the analyte that the specific binding molecule of the second binding guide conjugate to which the first binding conjugate corresponds interacts with,
wherein one guide compliment portion of the first half circle probe hybridizes to the guide oligonucleotide of the first binding guide conjugate and another guide compliment portion of the first half circle probe hybridizes to the guide oligonucleotide of the second binding guide conjugate,
wherein one guide compliment portion of the second half circle probe hybridizes to the guide oligonucleotide of the first binding guide conjugate and another guide compliment portion of the second half circle probe hybridizes to the guide oligonucleotide of the second binding guide conjugate, and
wherein the first and second half circle probes are ligated together to form the amplification target circle.

91. The method of claim 1, wherein two guide complement portions of two different half circle probes hybridize to the guide oligonucleotides, wherein one of the guide complement portions has a 5′ end and the other guide complement portion has a 3′ end.

92. The method of claim 91, wherein the 5′ end and the 3′ end are immediately adjacent when the two guide complement portions are hybridized to the guide oligonucleotide.

93. The method of claim 91, wherein the 5′ end and the 3′ end are separated by a central region when the two guide complement portions are hybridized to the guide oligonucleotide.

94. The method of claim 93, wherein step (b) further comprises, prior to incubating, bringing one or more gap oligonucleotides into contact with the binding guide conjugates, wherein each gap oligonucleotide comprises a single-stranded, linear DNA molecule, wherein each gap oligonucleotide is complementary all or a portion of the central region, wherein the gap oligonucleotides complementary to the central region are incorporated into the amplification target circles.

95. A method comprising:
(a) bringing into contact one or more analyte samples and one or more arrays, wherein each array comprises a set of analyte capture agents, a set of accessory molecules, or both, wherein each analyte capture agent interacts with an analyte directly or indirectly,
(b) prior to, simultaneous with, or following step (a), bringing into contact one or more of the arrays and one or more binding guide conjugates, wherein each binding guide conjugate comprises a specific binding molecule and a guide oligonucleotide, wherein each specific binding molecule interacts directly or indirectly with an analyte in the analyte sample, and
incubating the analyte samples and the binding guide conjugates under conditions that promote interaction of the specific binding molecules and the analytes,
(c) prior to, simultaneous with, or following steps (a) or (b), bringing into contact the binding guide conjugates and one or more half circle probes, wherein each half circle probe comprises a single-stranded DNA molecule comprising two guide complement portions, wherein each guide complement portion is complementary to at least one of the guide oligonucleotides, and
incubating the binding guide conjugates and the half circle probes under conditions that promote hybridization between the guide oligonucleotides and the half circle probes,
(d) following step (c) and prior to, simultaneous with, or following steps (a) or (b), incubating the binding guide conjugates and half circle probes under conditions that promote ligation of half circle probes to each other, thereby producing one or more amplification target circles, and
(e) following step (d), incubating the amplification target circles under conditions that promote replication of the amplification target circles, wherein replication of the amplification target circles results in the formation of tandem sequence DNA.

96. The method of claim 95, wherein each array comprises a set of analyte capture agents, and wherein each analyte capture agent is immobilized on a solid support in a different predefined region of the solid support.

97. The method of claim 96, wherein the distance between the different predefined regions of the solid support is fixed.

98. The method of claim 97, wherein the solid support comprises thin film, membrane, bottles, dishes, fibers, woven fibers, shaped polymers, particles, beads, microparticles, or a combination.

99. The method of claim 96, wherein the distance between at least two of the different predefined regions of the solid support is variable.

100. The method of claim 96, wherein the analyte capture agents are immobilized to the solid support at a density exceeding 400 different analyte capture agents per cubic centimeter.

101. The method of claim 96, wherein the analyte capture agents are peptides.

102. The method of claim 101, wherein each of the different peptides is at least 4 amino acids in length.

103. The method of claim 102, wherein each different peptide is from about 4 to about 20 amino acids in length.

104. The method of claim 102, wherein each different peptide is at least 10 amino acids in length.

105. The method of claim 102, wherein each different peptide is at least 20 amino acids in length.

106. The method of claim 96, wherein at least one array comprises at least 1,000 different analyte capture agents immobilized on the solid support.

107. The method of claim 96, wherein at least one array comprises at least 10,000 different analyte capture agents immobilized on the solid support.

108. The method of claim 96, wherein at least one array comprises at least 100,000 different analyte capture agents immobilized on the solid support.

109. The method of claim 96, wherein at least one array comprises at least 1,000,000 different analyte capture agents immobilized on the solid support.

110. The method of claim 96, wherein each of the different predefined regions is physically separated from each other of the different regions.

111. The method of claim 96, wherein the solid support comprises thin film, membrane, bottles, dishes, fibers, woven fibers, shaped polymers, particles, beads, microparticles, or a combination.

112. The method of claim 96, wherein the solid support comprises acrylamide, agarose, cellulose, nitrocellulose, glass, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, polypropylfumerate, collagen, glycosaminoglycans, or polyamino acids.

113. The method of claim 96, wherein the solid support is porous.

114. The method of claim 96, wherein the analyte capture agents in the different predefined regions are at least 20% pure.

115. The method of claim 96, wherein the analyte capture agents in the different predefined regions are at least 50% pure.

116. The method of claim 96, wherein the analyte capture agents in the different predefined regions are at least 80% pure.

117. The method of claim 96, wherein the analyte capture agents in the different predefined regions are at least 90% pure.

118. A method comprising:
(a) treating one or more analyte samples so that one or more analytes are modified,
(b) prior to, simultaneous with, or following step (a), bringing into contact one or more of the analyte samples and one or more binding guide conjugates, wherein each binding guide conjugate comprises a specific binding molecule and a guide oligonucleotide, wherein each specific binding molecule interacts directly or indirectly with an analyte in the analyte sample, and
incubating the analyte samples and the binding guide conjugates under conditions that promote interaction of the specific binding molecules and the analytes,
(c) prior to, simultaneous with, or following steps (a) or (b), bringing into contact the binding guide conjugates and one or more half circle probes, wherein each half circle probe comprises a single-stranded DNA molecule comprising two guide complement portions, wherein each guide complement portion is complementary to at least one of the guide oligonucleotides, and
incubating the binding guide conjugates and the half circle probes under conditions that promote hybridization between the guide oligonucleotides and the half circle probes,
(d) following step (c) and prior to, simultaneous with, or following steps (a) or (b), incubating the binding guide conjugates and half circle probes under conditions that promote ligation of half circle probes to each other, thereby producing one or more amplification target circles, and
(e) following step (d), incubating the amplification target circles under conditions that promote replication of the amplification target circles, wherein replication of the amplification target circles results in the formation of tandem sequence DNA.

119. The method of claim 118, wherein all of the analytes are modified by associating a modifying group to the analytes, wherein the modifying group is the same for all of the analytes, wherein all of the specific binding molecules interact with the modifying group.

120. A method comprising:
(a) bringing into contact one or more analyte samples and one or more arrays, wherein each array comprises a set of analyte capture agents, a set of accessory molecules, or both, wherein each analyte capture agent interacts with an analyte directly or indirectly,
(b) prior to, simultaneous with, or following step (a), bringing into contact one or more of the analyte samples and one or more binding guide conjugates, wherein each binding guide conjugate comprises a specific binding molecule and a guide oligonucleotide, wherein each specific binding molecule interacts directly or indirectly with an analyte in the analyte sample, and
incubating the analyte samples and the binding guide conjugates under conditions that promote interaction of the specific binding molecules and the analytes,
(c) prior to, simultaneous with, or following steps (a) or (b), bringing into contact the binding guide conjugates and one or more half circle probes, wherein each half circle probe comprises a single-stranded DNA molecule comprising two guide complement portions, wherein each guide complement portion is complementary to at least one of the guide oligonucleotides, and
incubating the binding guide conjugates and the half circle probes under conditions that promote hybridization between the guide oligonucleotides and the half circle probes,
(d) following step (c) and prior to, simultaneous with, or following steps (a) or (b), incubating the binding guide conjugates and half circle probes under conditions that promote ligation of half circle probes to each other, thereby producing one or more amplification target circles, and
(e) following step (d), incubating the amplification target circles under conditions that promote replication of the amplification target circles, wherein replication of the amplification target circles results in the formation of tandem sequence DNA.

121. A kit comprising
(a) a plurality of binding guide conjugates, wherein each binding guide conjugates comprises a specific binding molecule and a guide oligonucleotide, wherein each specific binding molecule interacts with an analyte directly or indirectly, and
(b) a plurality of half circle probes, wherein each half circle probe comprises a single-stranded DNA molecule comprising two guide complement portions, wherein each guide complement portion is complementary to at least one of the guide oligonucleotides.

122. The kit of claim 121, wherein the kit further comprises a plurality of analyte capture agents, and wherein each analyte capture agent interacts with an analyte directly or indirectly

123. The kit of claim 122, wherein the analyte capture agents are associated with a solid support.

124. The kit of claim 121, wherein each guide oligonucleotide is complementary to one of the guide complement portions of both half circle probes in a pair of half circle probes, wherein the guide complement portions of the same half circle probe are complementary to two different guide oligonucleotides,
wherein the two different guide oligonucleotides that each are complementary to a different one of the guide complement portions of the same half circle probe constitute a pair of guide oligonucleotides, wherein the binding guide conjugates that comprise the guide oligonucleotides in a pair of guide oligonucleotides constitute a pair of binding guide conjugates,
wherein one guide complement portion of each half circle probe in a pair of half circle probes is complementary to one of the guide oligonucleotides in a pair of guide oligonucleotides and the other guide complement portion of each half circle probe in the pair of half circle probes is complementary to the other guide oligonucleotide in the pair of guide oligonucleotides,
wherein the specific binding molecules of the binding guide conjugates in a pair of binding guide conjugates interact with the same analyte, and wherein both half circle probes in a pair of half circle probes are hybridized to both guide oligonucleotides in a pair of guide oligonucleotides.

125. The kit of claim 124, wherein each guide oligonucleotide is complementary to one of the guide complement portions of both half circle probes in a different pair of half circle probes.

126. The kit of claim 124, wherein the binding guide conjugates comprise a plurality of pairs of binding guide conjugates, wherein the guide oligonucleotides of the binding guide conjugates comprise a plurality of pairs of guide oligonucleotides, and wherein the half circle probes comprise a plurality of pairs of half circle probes.

127. The kit of claim 124, wherein the binding guide conjugates comprise a pair of binding guide conjugates, wherein the guide oligonucleotides of the binding guide conjugates comprise a pair of guide oligonucleotides, and wherein the half circle probes comprise a pair of half circle probes.

128. A method comprising:
(a) bringing into contact one or more analyte samples and one or more binding half circle conjugates, wherein each binding half circle conjugate comprises a specific binding molecule and a half circle probe, wherein each specific binding molecule interacts directly or indirectly with an analyte in the analyte sample, and wherein each half circle probe comprises a single-stranded DNA molecule comprising two guide complement portions, and
incubating the analyte samples and the binding half circle conjugates under conditions that promote interaction of the specific binding molecules and the analytes,
(b) prior to, simultaneous with, or following step (a), bringing into contact the binding half circle conjugates and one or more guide oligonucleotides, wherein each guide oligonucleotide is complementary to at least one of the guide complement portions of the half circle probes, and
incubating the binding half circle conjugates and the guide oligonucleotides under conditions that promote hybridization between the guide oligonucleotides and the half circle probes,
(c) following step (b) and prior to, simultaneous with, or following step (a), incubating the binding half circle conjugates and guide oligonucleotides under conditions that promote ligation of half circle probes to each other, thereby producing one or more amplification target circles, and
(d) following step (c), incubating the amplification target circles under conditions that promote replication of the amplification target circles, wherein replication of the amplification target circles results in the formation of tandem sequence DNA.

129. The method of claim 128, wherein following step (c) and prior to or simultaneous with step (d) the amplification target circles are disassociated from the specific binding molecules.

130. The method of claim 129, wherein the amplification target circles are disassociated from the specific binding molecules by cleaving covalent bonds.

131. The method of claim 129, wherein the amplification target circles are disassociated from the specific binding molecules by treating the amplification target circles with a reducing agent.

132. The method of claim 129, wherein the amplification target circles are disassociated from the specific binding molecules by heating the amplification target circles.

133. The method of claim 128, wherein the half circle probes are tethered to the specific binding molecules.

134. The method of claim 128, wherein the half circle probes are covalently coupled to the specific binding molecules.

135. The method of claim 134, wherein the half circle probes are covalently coupled to the specific binding molecules via a cleavable linker.

136. A method comprising:
(a) bringing into contact one or more analyte samples and one or more binding guide conjugates, wherein each binding guide conjugate comprises a specific binding molecule and a guide oligonucleotide, wherein each specific binding molecule interacts directly or indirectly with an analyte in the analyte sample, and
incubating the analyte samples and the binding guide conjugates under conditions that promote interaction of the specific binding molecules and the analytes,
(b) prior to, simultaneous with, or following step (a), bringing into contact the binding guide conjugates and one or more half circle probes, wherein each half circle probe comprises a single-stranded DNA molecule comprising two guide complement portions, wherein each guide complement portion is complementary to at least one of the guide oligonucleotides, and
incubating the binding guide conjugates and the half circle probes under conditions that promote hybridization between the guide oligonucleotides and the half circle probes,
(c) following step (b) and prior to, simultaneous with, or following step (a), incubating the binding guide conjugates and half circle probes under conditions that promote ligation of half circle probes to each other, thereby producing one or more amplification target circles, and
(d) following step (c), incubating the binding guide conjugates and amplification target circles under conditions that promote replication of the amplification target circles, wherein replication of the amplification target circles results in the formation of tandem sequence DNA.

137. A method comprising:
(a) bringing into contact one or more analyte samples and one or more binding guide conjugates, wherein each binding guide conjugate comprises a specific binding molecule and a guide oligonucleotide, wherein each specific binding molecule interacts directly or indirectly with an analyte in the analyte sample, and
incubating the analyte samples and the binding guide conjugates under conditions that promote interaction of the specific binding molecules and the analytes,
(b) prior to, simultaneous with, or following step (a), bringing into contact the binding guide conjugates and one or more half circle probes, wherein each half circle probe comprises a single-stranded DNA molecule comprising two guide complement portions,
wherein each guide complement portion is complementary to at least one of the guide oligonucleotides, wherein each guide oligonucleotide is complementary to one of the guide complement portions of both half circle probes in a pair of half circle probes, wherein the guide complement portions of the same half circle probe are complementary to two different guide oligonucleotides,
wherein the two different guide oligonucleotides that each are complementary to a different one of the guide complement portions of the same half circle probe constitute a pair of guide oligonucleotides, wherein the binding guide conjugates that comprise the guide oligonucleotides in a pair of guide oligonucleotides constitute a pair of binding guide conjugates,
wherein one guide complement portion of each half circle probe in a pair of half circle probes is complementary to one of the guide oligonucleotides in a pair of guide oligonucleotides and the other guide complement portion of each half circle probe in the pair of half circle probes is complementary to the other guide oligonucleotide in the pair of guide oligonucleotides,
wherein both half circle probes in a pair of half circle probes are hybridized to both guide oligonucleotides in a pair of guide oligonucleotides, and
incubating the binding guide conjugates and the half circle probes under conditions that promote hybridization between the guide oligonucleotides and the half circle probes,
(c) following step (b) and prior to, simultaneous with, or following step (a), incubating the binding guide conjugates and half circle probes under conditions that promote ligation of half circle probes in pairs of half circle probes to each other, thereby producing one or more amplification target circles.

138. A method comprising:
(a) bringing into contact one or more analyte samples and one or more binding guide conjugates, wherein each binding guide conjugate comprises a specific binding molecule and a guide oligonucleotide, wherein each specific binding molecule interacts directly or indirectly with an analyte in the analyte sample, and
incubating the analyte samples and the binding guide conjugates under conditions that promote interaction of the specific binding molecules and the analytes,
(b) prior to, simultaneous with, or following step (a), bringing into contact the binding guide conjugates and one or more half circle probes, wherein each half circle probe comprises a single-stranded DNA molecule comprising two guide complement portions, wherein each guide complement portion is complementary to at least one of the guide oligonucleotides, and
incubating the binding guide conjugates and the half circle probes under conditions that promote hybridization between the guide oligonucleotides and the half circle probes,
(c) following steps (a) and (b), incubating the binding guide conjugates and half circle probes under conditions that promote ligation of half circle probes to each other, thereby producing one or more amplification target circles.


SEQUENCE LISTING (6 sequences)

SEQ ID NO: 1 - DNA, 51 bp, Artificial Sequence
  Description of Artificial Sequence /note=synthetic construct
daaaaaaaaaaaaaaaaaaaaaaaaaaaaaagttctatgatgatgagagtc

SEQ ID NO: 2 - DNA, 51 bp, Artificial Sequence
  Description of Artificial Sequence /note=synthetic construct
daaaaaaaaaaaaaaaaaaaaaaaaaaaaaacgcgacggccgataacagag

SEQ ID NO: 3 - DNA, 40 bp, Artificial Sequence
  Description of Artificial Sequence /note=synthetic construct
datcatagaacacgaacagctggtcatctgctctgttatc

SEQ ID NO: 4 - DNA, 40 bp, Artificial Sequence
  Description of Artificial Sequence /note=synthetic construct
dggccgtcgcgcagacacgatagatgaggcgactctcatc

SEQ ID NO: 5 - DNA, 21 bp, Artificial Sequence
  Description of Artificial Sequence /note=synthetic construct
dtctatgatgatgagagtcgc

SEQ ID NO: 6 - DNA, 79 bp, Artificial Sequence
  Description of Artificial Sequence /note=synthetic construct
datcatagaacacgaacagctggtcatctgctctgttatcggccgtcgcgcagacacgatagatgaggcgactctcatc